(19) United States
(12) Patent Application Publication
(10) Pub. No.: US 2026/0232843 A1
(43) Pub. Date: Aug. 13, 2026

(54) COMPOSITIONS TARGETING KLKB1 AND METHODS OF USE THEREOF

(71) Applicant: POSEIDA THERAPEUTICS, INC., San Diego, CA (US)

(72) Inventors: Blair B. MADISON, San Diego, CA (US); Oscar ALVAREZ, San Diego, CA (US)

(73) Assignee: POSEIDA THERAPEUTICS, INC., San Diego, CA (US)

(21) Appl. No.: 19/665,837

(22) Filed: May 1, 2026

Related U.S. Application Data
(63) Continuation of application No. PCT/US2024/054167, filed on Nov. 1, 2024.
(60) Provisional application No. 63/595,565, filed on Nov. 2, 2023.
(60) Provisional application No. 63/711,106, filed on Oct. 23, 2024.

(51) Int. Cl. A61K 48/00; A61K 9/1271; A61K 9/51; A61P 3/00; C12N 9/22; C12N 9/64; C12N 15/11; C12N 15/88

(52) U.S. Cl. CPC A61K 48/005; A61K 9/1271; A61K 9/5123; A61P 3/00; C12N 9/226; C12N 9/6445; C12N 15/111; C12N 15/88; C07K 2319/80; C12N 2310/20; C12N 2310/315; C12N 2310/321; C12Y 301/21004; C12Y 304/21034

(57) ABSTRACT

Disclosed are methods and compositions for functional genetic modifications at selected genomic sites such as KLKB1 gene. Also provided are cell populations, which comprise the functional genetic modification at one or more selected gene loci.

### CROSS-REFERENCE TO RELATED APPLICATIONS

This application is a continuation of International Patent Application No. PCT/US2024/054167, filed Nov. 1, 2024, which claims the benefit of U.S. Provisional Application No. 63/595,565 filed Nov. 2, 2023 and U.S. Provisional Application No. 63/711,106 filed Oct. 23, 2024. Each of the foregoing applications is herein incorporated by reference in its entirety.

### INCORPORATION-BY-REFERENCE OF SEQUENCE LISTING

The instant application contains a Sequence Listing which has been submitted electronically in XML format and is herein incorporated by reference in its entirety. Said XML copy, created on May 1, 2026, is named 30006-030220-US2-SL.xml and is 80,924 bytes in size.

### FIELD OF THE DISCLOSURE

The disclosure is directed to the field of genetic editing and genomic engineering. More particularly, the present disclosure is directed to compositions and methods for targeted genetic modification and modulating expression of a target nucleic acid sequence and applications thereof.

### BACKGROUND

Genome editing refers to strategies and techniques for the targeted, specific modification of the genetic information (genome) of living organisms. Genome editing is an active field of research because of the wide range of possible applications, particularly in the area of human health, e.g., to correct a gene carrying a harmful mutation or to explore the function of a gene. Early technologies developed to insert a transgene into a living cell were often limited by the random nature of the insertion location of the new sequence into the genome. Common genome editing strategies allow a specific area of the DNA to be modified, thereby increasing the precision of the correction or insertion compared to earlier technologies. While these platforms offer a greater degree of reproducibility and a decreased level of unintended effects from random insertions and deletions in the genome, limitations remain.

Plasma kallikrein is a serine protease component of the contact system and a potential drug target for different inflammatory, cardiovascular, infectious (sepsis), and oncology diseases (Sainz I. M. et al., Thromb Haemost 98, 77-83, 2007). A genetic deficiency in the C1-inhibitor protein (C1-INH), the major natural inhibitor of plasma kallikrein and the kallikrein-bradykinin pathway, leads to hereditary angioedema (HAE). Patients with HAE suffer from acute attacks of painful edema 2-4 times per month, often precipitated by unknown triggers (Zuraw B. L. et al., N Engl J Med 359, 1027-1036, 2008). The current standard of care consists of C1-esterase inhibitors or plasma kallikrein inhibitors, which are transient treatments. There is a need for improved therapeutic compositions and methods for the treatment of HAE. There is a need to develop gene editing platforms with superior efficacy in genome editing for the treatment of HAE.

### SUMMARY

This disclosure provides compositions and methods for genomic editing of a KLKB1 gene. In some aspects, the methods and compositions are for the treatment of hereditary angioedema. In one aspect, the present disclosure provides compositions comprising: (a) a first guide RNA (gRNA) comprising a first targeting sequence set forth in SEQ ID NOs: 1-17, 49, 50, or 55; (b) a second gRNA comprising a second targeting sequence set forth in SEQ ID NOs: 18-34, 51, 52, or 56; (c) a first polynucleotide encoding a first fusion protein, wherein the first fusion protein comprises a first inactivated Cas9 domain, or nuclease domain thereof, and a first Clo051 domain, or nuclease domain thereof, and (d) a second polynucleotide encoding a second fusion protein, wherein the second fusion protein comprises a second inactivated Cas9 domain, or nuclease domain thereof, and a second Clo051 domain, or nuclease domain thereof.

In some aspects, the C-terminus of the first inactivated Cas9 domain, the second inactivated Cas9 domain, or both the first and second inactivated Cas9 domain, or nuclease domain thereof, and the N-terminus of the first Clo051 domain, the second Clo051 domain, or both the first and second Clo051 domain, or nuclease domain thereof, are connected by a linker comprising the sequence set forth in SEQ ID NO: 57.

The present disclosure provides compositions comprising: a) a first guide RNA (gRNA) and a fusion protein or a polynucleotide encoding a first fusion protein comprising: a mutant Cas9 (dCas9) polypeptide or an inactivated nuclease domain thereof and a Clo051 polypeptide or a nuclease domain thereof, configured to form a complex with the first gRNA, and b) a second gRNA and a fusion protein or a polynucleotide encoding a second fusion protein comprising: a mutant Cas9 (dCas9) polypeptide or an inactivated nuclease domain thereof and a Clo051 polypeptide or a nuclease domain thereof, configured to form a complex with the second gRNA; wherein the first gRNA comprises a first targeting sequence comprising the nucleic acid sequence selected from SEQ ID NOs: 1-17, 49, 50 or 55; and the second gRNA comprises a second targeting sequence comprising the nucleic acid sequence of SEQ ID NOs: 18-34, 51, 52, and 56.

In some embodiments of the composition, i) the first gRNA comprises a first targeting sequence comprising the nucleic acid sequence of SEQ ID NO: 1 and the second gRNA comprises a second targeting sequence comprising the nucleic acid sequence of SEQ ID NO: 18, ii) the first gRNA comprises a first targeting sequence comprising the nucleic acid sequence of SEQ ID NO: 2 and the second gRNA comprises a second targeting sequence comprising the nucleic acid sequence of SEQ ID NO: 19, iii) the first gRNA comprises a first targeting sequence comprising the nucleic acid sequence of SEQ ID NO: 3 and the second gRNA comprises a second targeting sequence comprising the nucleic acid sequence of SEQ ID NO: 20, iv) the first gRNA comprises a first targeting sequence comprising the nucleic acid sequence of SEQ ID NO: 4 and the second gRNA comprises a second targeting sequence comprising the nucleic acid sequence of SEQ ID NO: 21, v) the first gRNA comprises a first targeting sequence comprising the nucleic acid sequence of SEQ ID NO: 5 and the second gRNA comprises a second targeting sequence comprising the nucleic acid sequence of SEQ ID NO: 22, vi) the first gRNA comprises a first targeting sequence comprising the nucleic acid sequence of SEQ ID NO: 6 and the second gRNA comprises a second targeting sequence comprising the nucleic acid sequence of SEQ ID NO: 23, vii) the first gRNA comprises a first targeting sequence comprising the nucleic acid sequence of SEQ ID NO: 7 and the second gRNA comprises a second targeting sequence comprising the nucleic acid sequence of SEQ ID NO: 24, viii) the first gRNA comprises a first targeting sequence comprising the nucleic acid sequence of SEQ ID NO: 8 and the second gRNA comprises a second targeting sequence comprising the nucleic acid sequence of SEQ ID NO: 25, ix) the first gRNA comprises a first targeting sequence comprising the nucleic acid sequence of SEQ ID NO: 9 and the second gRNA comprises a second targeting sequence comprising the nucleic acid sequence of SEQ ID NO: 26, x) the first gRNA comprises a first targeting sequence comprising the nucleic acid sequence of SEQ ID NO: 10 and the second gRNA comprises a second targeting sequence comprising the nucleic acid sequence of SEQ ID NO: 27, xi) the first gRNA comprises a first targeting sequence comprising the nucleic acid sequence of SEQ ID NO: 11 and the second gRNA comprises a second targeting sequence comprising the nucleic acid sequence of SEQ ID NO: 28, xii) the first gRNA comprises a first targeting sequence comprising the nucleic acid sequence of SEQ ID NO: 12 and the second gRNA comprises a second targeting sequence comprising the nucleic acid sequence of SEQ ID NO: 29, xiii) the first gRNA comprises a first targeting sequence comprising the nucleic acid sequence of SEQ ID NO: 13 and the second gRNA comprises a second targeting sequence comprising the nucleic acid sequence of SEQ ID NO: 30, xiv) the first gRNA comprises a first targeting sequence comprising the nucleic acid sequence of SEQ ID NO: 14 and the second gRNA comprises a second targeting sequence comprising the nucleic acid sequence of SEQ ID NO: 31, xv) the first gRNA comprises a first targeting sequence comprising the nucleic acid sequence of SEQ ID NO: 15 and the second gRNA comprises a second targeting sequence comprising the nucleic acid sequence of SEQ ID NO: 32, xvi) the first gRNA comprises a first targeting sequence comprising the nucleic acid sequence of SEQ ID NO: 16 and the second gRNA comprises a second targeting sequence comprising the nucleic acid sequence of SEQ ID NO: 33, xvii) the first gRNA comprises a first targeting sequence comprising the nucleic acid sequence of SEQ ID NO: 17 and the second gRNA comprises a second targeting sequence comprising the nucleic acid sequence of SEQ ID NO: 34, xviii) the first gRNA comprises a first targeting sequence comprising the nucleic acid sequence of SEQ ID NO: 49 and the second gRNA comprises a second targeting sequence comprising the nucleic acid sequence of SEQ ID NO: 51, xix) the first gRNA comprises a first targeting sequence comprising the nucleic acid sequence of SEQ ID NO: 50 and the second gRNA comprises a second targeting sequence comprising the nucleic acid sequence of SEQ ID NO: 52, or xx) the first gRNA comprises a first targeting sequence comprising the nucleic acid sequence of SEQ ID NO: 55 and the second gRNA comprises a second targeting sequence comprising the nucleic acid sequence of SEQ ID NO: 57.

In some aspects, the first fusion protein, the second fusion protein or both the first and the second fusion protein comprise the amino acid sequence of SEQ ID NO: 35. In some aspects, the first fusion protein, the second fusion protein, or both the first and the second fusion protein comprise the amino acid sequence of SEQ ID NO: 38.

In some aspects, the first fusion protein comprises the amino acid sequence set forth in SEQ ID NO: 35 or SEQ ID NO: 38, and/or the second fusion protein comprises the amino acid sequence set forth in SEQ ID NO: 35 or SEQ ID NO: 38.

In some aspects, the polynucleotide encoding the first fusion protein, the second fusion protein, or both the first and the second fusion protein is an mRNA. In some aspects, the polynucleotide comprises the nucleic acid sequence of SEQ ID NO: 36. In some aspects, the polynucleotide comprises the nucleic acid sequence of SEQ ID NO: 39. In some aspects, the mRNA comprises a 5′-cap.

In some aspects, the first polynucleotide is an mRNA comprising the sequence set forth in SEQ ID NO: 36 or SEQ ID NO: 39, and/or the second polynucleotide is an mRNA comprising the sequence set forth in SEQ ID NO: 36 or SEQ ID NO: 39.

In some aspects, the dCas9 is derived from a S. pyogenes Cas9 polypeptide. In some aspects, the gRNA comprises a guide sequence and a scaffold sequence isolated from Streptococcus pyogenes.

In some aspects the first inactivated Cas9 domain is derived from a Streptococcus pyogenes Cas9 polypeptide, and/or the second inactivated Cas9 domain is derived from a Streptococcus pyogenes Cas9 polypeptide. In some aspects, the first gRNA comprises a spacer and a scaffold sequence isolated from Streptococcus pyogenes, and/or the second gRNA comprises a spacer and a scaffold sequence isolated from Streptococcus pyogenes.

In some aspects, the scaffold sequence comprises the nucleic acid sequence of SEQ ID NO: 48.

In some aspects, the C-terminus of the dCas9, or inactivated nuclease domain thereof, is joined to the N-terminus of the Clo051 polypeptide or nuclease domain thereof via the peptide linker sequence of GGGGS (SEQ ID NO: 57). In some embodiments, the C-terminus of the first fusion protein further comprises a linker comprising the sequence set forth in SEQ ID NO: 57, and/or the second fusion protein further comprises a linker comprising the sequence set forth in SEQ ID NO: 57.

In some aspects, the first gRNA, the second gRNA, or both the first gRNA and the second gRNA comprises one or more chemical modifications of a ribonucleotide, a ribonucleotide base, or a phosphodiester bond. In some aspects, the one or more chemical modifications comprises at least one chemically modified phosphodiester bond. In some aspects, the at least one chemically modified phosphodiester bond is a phosphorothioate bond.

In some aspects, the first gRNA comprises one or more chemical modifications of a ribonucleotide, a ribonucleotide base, or a phosphodiester bond, and/or the second gRNA comprises one or more chemical modifications of a ribonucleotide, a ribonucleotide base, or a phosphodiester bond. In some aspects, the chemical modification comprises a phosphorothioate bond.

In some aspects, at least two consecutive phosphorothioate bonds are at the 5′-terminus and/or the 3′-terminus of the first gRNA, the second gRNA or both the first gRNA and the second gRNA. In some aspects, at least one 2′ O-Me chemical modification is at the 5′-terminus and/or the 3′-terminus of the first gRNA, the second gRNA or both the first gRNA and the second gRNA.

In some aspects, the 5′- and/or 3′-terminus of the first gRNA comprises at least two consecutive phosphorothioate bonds, and/or the 5′- and/or 3′-terminus of the second gRNA comprises at least two consecutive phosphorothioate bonds.

In some aspects, the 5′- and/or 3′-terminus of the first gRNA comprises at least one 2′ O-Me chemical modification, and/or wherein the 5′- and/or 3′-terminus of the second gRNA comprises at least one 2′ O-Me chemical modification.

The disclosure provides a composition comprising: i) a first gRNA comprising a first targeting sequence comprising the nucleic acid sequence of SEQ ID NO: 1, a second gRNA comprising a second targeting sequence comprising the nucleic acid sequence of SEQ ID NO: 18, and a polynucleotide encoding a fusion protein comprising the amino acid sequence of SEQ ID NO: 35, ii) a first gRNA comprising a first targeting sequence comprising the nucleic acid sequence of SEQ ID NO: 2, a second gRNA comprising a second targeting sequence comprising the nucleic acid sequence of SEQ ID NO: 19, and a polynucleotide encoding a fusion protein comprising the amino acid sequence of SEQ ID NO: 35, iii) a first gRNA comprising a first targeting sequence comprising the nucleic acid sequence of SEQ ID NO: 3, a second gRNA comprising a second targeting sequence comprising the nucleic acid sequence of SEQ ID NO: 20, and a polynucleotide encoding a fusion protein comprising the amino acid sequence of SEQ ID NO: 35, iv) a first gRNA comprising a first targeting sequence comprising the nucleic acid sequence of SEQ ID NO: 4, a second gRNA comprising a second targeting sequence comprising the nucleic acid sequence of SEQ ID NO: 21, and a polynucleotide encoding a fusion protein comprising the amino acid sequence of SEQ ID NO: 35, v) a first gRNA comprising a first targeting sequence comprising the nucleic acid sequence of SEQ ID NO: 5, a second gRNA comprising a second targeting sequence comprising the nucleic acid sequence of SEQ ID NO: 22, and a polynucleotide encoding a fusion protein comprising the amino acid sequence of SEQ ID NO: 35, vi) a first gRNA comprising a first targeting sequence comprising the nucleic acid sequence of SEQ ID NO: 6, a second gRNA comprising a second targeting sequence comprising the nucleic acid sequence of SEQ ID NO: 23, and a polynucleotide encoding a fusion protein comprising the amino acid sequence of SEQ ID NO: 35, vii) a first gRNA comprising a first targeting sequence comprising the nucleic acid sequence of SEQ ID NO: 7, a second gRNA comprising a second targeting sequence comprising the nucleic acid sequence of SEQ ID NO: 24, and a polynucleotide encoding a fusion protein comprising the amino acid sequence of SEQ ID NO: 35, viii) a first gRNA comprising a first targeting sequence comprising the nucleic acid sequence of SEQ ID NO: 8, a second gRNA comprising a second targeting sequence comprising the nucleic acid sequence of SEQ ID NO: 25, and a polynucleotide encoding a fusion protein comprising the amino acid sequence of SEQ ID NO: 35, ix) a first gRNA comprising a first targeting sequence comprising the nucleic acid sequence of SEQ ID NO: 9, a second gRNA comprising a second targeting sequence comprising the nucleic acid sequence of SEQ ID NO: 26, and a polynucleotide encoding a fusion protein comprising the amino acid sequence of SEQ ID NO: 35, x) a first gRNA comprising a first targeting sequence comprising the nucleic acid sequence of SEQ ID NO: 10, a second gRNA comprising a second targeting sequence comprising the nucleic acid sequence of SEQ ID NO: 27, and a polynucleotide encoding a fusion protein comprising the amino acid sequence of SEQ ID NO: 35, xi) a first gRNA comprising a first targeting sequence comprising the nucleic acid sequence of SEQ ID NO: 11, a second gRNA comprising a second targeting sequence comprising the nucleic acid sequence of SEQ ID NO: 28, and a polynucleotide encoding a fusion protein comprising the amino acid sequence of SEQ ID NO: 35, xii) a first gRNA comprising a first targeting sequence comprising the nucleic acid sequence of SEQ ID NO: 12, a second gRNA comprising a second targeting sequence comprising the nucleic acid sequence of SEQ ID NO: 29, and a polynucleotide encoding a fusion protein comprising the amino acid sequence of SEQ ID NO: 35, xiii) a first gRNA comprising a first targeting sequence comprising the nucleic acid sequence of SEQ ID NO: 13, a second gRNA comprising a second targeting sequence comprising the nucleic acid sequence of SEQ ID NO: 30, and a polynucleotide encoding a fusion protein comprising the amino acid sequence of SEQ ID NO: 35, xiv) a first gRNA comprising a first targeting sequence comprising the nucleic acid sequence of SEQ ID NO: 14, a second gRNA comprising a second targeting sequence comprising the nucleic acid sequence of SEQ ID NO: 31, and a polynucleotide encoding a fusion protein comprising the amino acid sequence of SEQ ID NO: 35, xv) a first gRNA comprising a first targeting sequence comprising the nucleic acid sequence of SEQ ID NO: 15, a second gRNA comprising a second targeting sequence comprising the nucleic acid sequence of SEQ ID NO: 32, and a polynucleotide encoding a fusion protein comprising the amino acid sequence of SEQ ID NO: 35, xvi) a first gRNA comprising a first targeting sequence comprising the nucleic acid sequence of SEQ ID NO: 16, a second gRNA comprising a second targeting sequence comprising the nucleic acid sequence of SEQ ID NO: 33, and a polynucleotide encoding a fusion protein comprising the amino acid sequence of SEQ ID NO: 35, xvii) a first gRNA comprising a first targeting sequence comprising the nucleic acid sequence of SEQ ID NO: 17, a second gRNA comprising a second targeting sequence comprising the nucleic acid sequence of SEQ ID NO: 34, and a polynucleotide encoding a fusion protein comprising the amino acid sequence of SEQ ID NO: 35, xviii) a first gRNA comprising a first targeting sequence comprising the nucleic acid sequence of SEQ ID NO: 49, a second gRNA comprising a second targeting sequence comprising the nucleic acid sequence of SEQ ID NO: 51, and a polynucleotide encoding a fusion protein comprising the amino acid sequence of SEQ ID NO: 35, xix) a first gRNA comprising a first targeting sequence comprising the nucleic acid sequence of SEQ ID NO: 50, a second gRNA comprising a second targeting sequence comprising the nucleic acid sequence of SEQ ID NO: 52, and a polynucleotide encoding a fusion protein comprising the amino acid sequence of SEQ ID NO: 35, xx) a first gRNA comprising a first targeting sequence comprising the nucleic acid sequence of SEQ ID NO: 55, a second gRNA comprising a second targeting sequence comprising the nucleic acid sequence of SEQ ID NO: 56, and a polynucleotide encoding a fusion protein comprising the amino acid sequence of SEQ ID NO: 35, xxi) a first gRNA comprising a first targeting sequence comprising the nucleic acid sequence of SEQ ID NO: 1, a second gRNA comprising a second targeting sequence comprising the nucleic acid sequence of SEQ ID NO: 18, and a polynucleotide encoding a fusion protein comprising the amino acid sequence of SEQ ID NO: 38, xxii) a first gRNA comprising a first targeting sequence comprising the nucleic acid sequence of SEQ ID NO: 2, a second gRNA comprising a second targeting sequence comprising the nucleic acid sequence of SEQ ID NO: 19, and a polynucleotide encoding a fusion protein comprising the amino acid sequence of SEQ ID NO: 38, xxiii) a first gRNA comprising a first targeting sequence comprising the nucleic acid sequence of SEQ ID NO: 3, a second gRNA comprising a second targeting sequence comprising the nucleic acid sequence of SEQ ID NO: 20, and a polynucleotide encoding a fusion protein comprising the amino acid sequence of SEQ ID NO: 38, xxiv) a first gRNA comprising a first targeting sequence comprising the nucleic acid sequence of SEQ ID NO: 4, a second gRNA comprising a second targeting sequence comprising the nucleic acid sequence of SEQ ID NO: 21, and a polynucleotide encoding a fusion protein comprising the amino acid sequence of SEQ ID NO: 38, xxv) a first gRNA comprising a first targeting sequence comprising the nucleic acid sequence of SEQ ID NO: 5, a second gRNA comprising a second targeting sequence comprising the nucleic acid sequence of SEQ ID NO: 22, and a polynucleotide encoding a fusion protein comprising the amino acid sequence of SEQ ID NO: 38, xxvi) a first gRNA comprising a first targeting sequence comprising the nucleic acid sequence of SEQ ID NO: 6, a second gRNA comprising a second targeting sequence comprising the nucleic acid sequence of SEQ ID NO: 23, and a polynucleotide encoding a fusion protein comprising the amino acid sequence of SEQ ID NO: 38, xxvii) a first gRNA comprising a first targeting sequence comprising the nucleic acid sequence of SEQ ID NO: 7, a second gRNA comprising a second targeting sequence comprising the nucleic acid sequence of SEQ ID NO: 24, and a polynucleotide encoding a fusion protein comprising the amino acid sequence of SEQ ID NO: 38, xxviii) a first gRNA comprising a first targeting sequence comprising the nucleic acid sequence of SEQ ID NO: 8, a second gRNA comprising a second targeting sequence comprising the nucleic acid sequence of SEQ ID NO: 25, and a polynucleotide encoding a fusion protein comprising the amino acid sequence of SEQ ID NO: 38, xxix) a first gRNA comprising a first targeting sequence comprising the nucleic acid sequence of SEQ ID NO: 9, a second gRNA comprising a second targeting sequence comprising the nucleic acid sequence of SEQ ID NO: 26, and a polynucleotide encoding a fusion protein comprising the amino acid sequence of SEQ ID NO: 38, xxx) a first gRNA comprising a first targeting sequence comprising the nucleic acid sequence of SEQ ID NO: 10, a second gRNA comprising a second targeting sequence comprising the nucleic acid sequence of SEQ ID NO: 27, and a polynucleotide encoding a fusion protein comprising the amino acid sequence of SEQ ID NO: 38, xxxi) a first gRNA comprising a first targeting sequence comprising the nucleic acid sequence of SEQ ID NO: 11, a second gRNA comprising a second targeting sequence comprising the nucleic acid sequence of SEQ ID NO: 28, and a polynucleotide encoding a fusion protein comprising the amino acid sequence of SEQ ID NO: 38, xxxii) a first gRNA comprising a first targeting sequence comprising the nucleic acid sequence of SEQ ID NO: 12, a second gRNA comprising a second targeting sequence comprising the nucleic acid sequence of SEQ ID NO: 29, and a polynucleotide encoding a fusion protein comprising the amino acid sequence of SEQ ID NO: 38, xxxiii) a first gRNA comprising a first targeting sequence comprising the nucleic acid sequence of SEQ ID NO: 13, a second gRNA comprising a second targeting sequence comprising the nucleic acid sequence of SEQ ID NO: 30, and a polynucleotide encoding a fusion protein comprising the amino acid sequence of SEQ ID NO: 38, xxxiv) a first gRNA comprising a first targeting sequence comprising the nucleic acid sequence of SEQ ID NO: 14, a second gRNA comprising a second targeting sequence comprising the nucleic acid sequence of SEQ ID NO: 31, and a polynucleotide encoding a fusion protein comprising the amino acid sequence of SEQ ID NO: 38, xxxv) a first gRNA comprising a first targeting sequence comprising the nucleic acid sequence of SEQ ID NO: 15, a second gRNA comprising a second targeting sequence comprising the nucleic acid sequence of SEQ ID NO: 32, and a polynucleotide encoding a fusion protein comprising the amino acid sequence of SEQ ID NO: 38, xxxvi) a first gRNA comprising a first targeting sequence comprising the nucleic acid sequence of SEQ ID NO: 16, a second gRNA comprising a second targeting sequence comprising the nucleic acid sequence of SEQ ID NO: 33, and a polynucleotide encoding a fusion protein comprising the amino acid sequence of SEQ ID NO: 38, xxxvii) a first gRNA comprising a first targeting sequence comprising the nucleic acid sequence of SEQ ID NO: 17, a second gRNA comprising a second targeting sequence comprising the nucleic acid sequence of SEQ ID NO: 34, and a polynucleotide encoding a fusion protein comprising the amino acid sequence of SEQ ID NO: 38, xxxviii) a first gRNA comprising a first targeting sequence comprising the nucleic acid sequence of SEQ ID NO: 49, a second gRNA comprising a second targeting sequence comprising the nucleic acid sequence of SEQ ID NO: 51, and a polynucleotide encoding a fusion protein comprising the amino acid sequence of SEQ ID NO: 38, xxxix) a first gRNA comprising a first targeting sequence comprising the nucleic acid sequence of SEQ ID NO: 50, a second gRNA comprising a second targeting sequence comprising the nucleic acid sequence of SEQ ID NO: 52, and a polynucleotide encoding a fusion protein comprising the amino acid sequence of SEQ ID NO: 38, or xL) a first gRNA comprising a first targeting sequence comprising the nucleic acid sequence of SEQ ID NO: 55, a second gRNA comprising a second targeting sequence comprising the nucleic acid sequence of SEQ ID NO: 56, and a polynucleotide encoding a fusion protein comprising the amino acid sequence of SEQ ID NO: 38.

In some aspects, the composition is encapsulated in at least one Lipid Nanoparticle (LNLP).

In some aspects, the composition is encapsulated in at least one LNP comprising: about 54% of SS-OP by moles, about 35% of cholesterol by moles, about 5% of DOPC by moles, about 5% of DSPC by moles, and about 1% of DMG-PEG2000 by moles; or about 54% of SS-OP by moles, about 35% of cholesterol by moles, about 10% of DOPE by moles, and about 1% of DMG-PEG2000 by moles, wherein the ratio of lipid to RNA molecule in the at least one nanoparticle is about 100:1 (w/w) and the total lipid of 25 nM.

In some aspects, the composition is encapsulated in at least one LNP comprising: about 50% COMPOUND NO. 1 by moles, about 50% COMPOUND NO. 1 by moles, about 10% DOPC by moles, about 38.5% cholesterol by moles, and about 1.5% DMG-PEG2000 by moles; or about 40% COMPOUND NO. 1 by moles, about 10% DOPC by moles, about 48.5% cholesterol by moles, and about 1.5% DMG-PEG2000 by moles, wherein the ratio of lipid to RNA molecule in the at least one nanoparticle is about 80:1 (w/w) or about 60:1, or about 50:1 and wherein COMPOUND NO. 1 comprises the following structure:

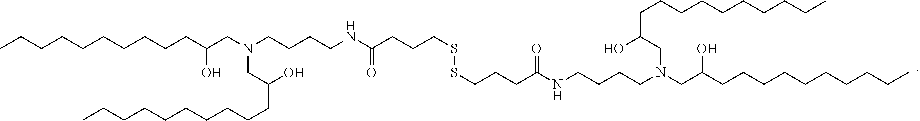 [SMILES: CCCCCCCCCCC(O)CN(CCCCNC(=O)CCCSSCCCC(=O)NCCCCN(CC(O)CCCCCCCCCC)CC(O)CCCCCCCCCC)CC(O)CCCCCCCCCC]

In some aspects, The composition of any one of claims 1-18, wherein the composition is encapsulated in at least one LNP comprising: about 50% COMPOUND NO. 37 by moles, about 10% DSPC by moles, about 38.5% cholesterol by moles, and about 1.5% DMG-PEG2000 by moles; or about 50% COMPOUND NO. 37 by moles, about 10% DOPC by moles, about 38% cholesterol by moles, and about 2% DMG-PEG2000 by moles, or about 50% COMPOUND NO. 37 by moles, about 10% DSPC by moles, about 38% cholesterol by moles, and about 2% DMG-PEG2000 by moles, or about 50% COMPOUND NO. 37 by moles, about 5% DSPC by moles, about 42% cholesterol by moles, and about 3% DMG-PEG2000 by moles, or about 50% COMPOUND NO. 37 by moles, about 10% DPPC by moles, about 38% cholesterol by moles, and about 2% DMG-PEG2000 by moles, wherein the ratio of lipid to RNA molecule in the at least one nanoparticle is about 50:1 (w/w), wherein the lipid nanoparticle further comprises at least one mRNA molecule and wherein COMPOUND NO. 37 comprises the following structure:

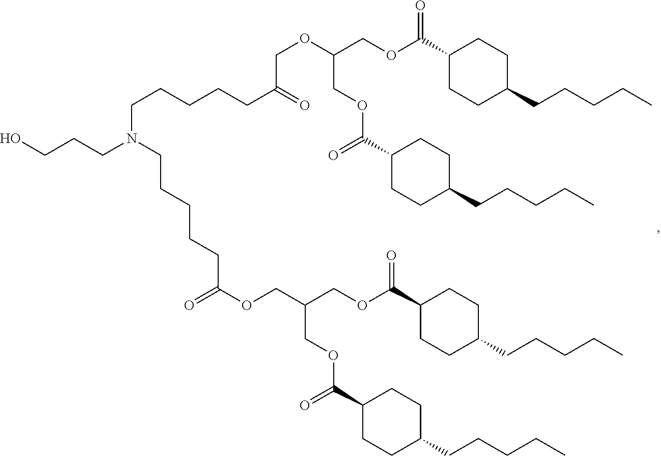 [SMILES: CCCCC[C@H]1CC[C@H](C(=O)OCC(COC(=O)CCCCCN(CCCO)CCCCCC(=O)COC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1]

In some aspects, the composition is for use in modifying a KLKB1 gene.

The disclosure provides a method of modifying a population of cells comprising contacting the population of cells with any one of the compositions of the disclosure, wherein the first gRNA forms a complex with the first targeting sequence and the first fusion protein, and the second gRNA forms a complex with the second targeting sequence and the second fusion protein, thereby generating an indel between the first targeting sequence and the second targeting sequence and producing a modified population of cells.

The disclosure provides methods of modifying a population of cells comprising contacting the population of cells with a composition of the present disclosure, wherein the first and second fusion proteins are expressed by each cell of the population, wherein the first gRNA is bound to the first fusion protein and the second gRNA is bound to the second fusion protein, wherein the first gRNA specifically binds to a first strand of a first double-stranded DNA target sequence in each cell of the population, and wherein the second gRNA specifically binds to a second strand of a second double-stranded DNA target sequence in each cell of the population.

In some aspects, the first fusion protein and the second fusion protein introduces a modification into the genome of one or more cells in the population. In some aspects, the modification is an insertion or deletion (indel) between the first double-stranded DNA target sequence and the second double-stranded DNA target sequence. In some aspects, the indel causes inactivation of a KLKB1 gene.

The disclosure provides populations of cells modified according to the methods of the present disclosure. In some aspects, the modified population of cells has about a reduced level of KLKB1 protein expression relative to an unmodified population of cells. In some aspects, the modified population of cells has at least a 50% reduction of KLKB1 protein expression relative to an unmodified population of cells. In some aspects, at least 20% of the cells in the population of modified cells include an indel at the KLKB1 locus. The disclosure provides a population of cells modified according to any one of the methods of the disclosure.

The disclosure provides a method of treating a Hereditary Angioedema in a subject in need thereof, comprising administering to a subject a therapeutically effective amount of one or more compositions of the disclosure. In some aspects, the Hereditary Angioedema is Hereditary Angioedema Type 1 or Hereditary Angioedema Type 2.

The disclosure provides the use of a composition of the present disclosure or a population of cells produced by a method of the present disclosure for the manufacture of a medicament for the treatment of Hereditary Angioedema. In some aspects, the Hereditary Angioedema is Hereditary Angioedema Type 1 or Hereditary Angioedema Type 2.

All documents cited herein, including any cross referenced or related patents or applications are hereby incorporated herein by reference in its entirety for all purposes, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

### BRIEF DESCRIPTION OF THE DRAWINGS

FIG. 1 is a schematic illustrating the KLKB1 target gene including its two different mRNA transcripts with individual peptides. gRNAs were designed to target exons 6-12 (boxed).

FIG. 2 is a graph showing indels in HUH7 cells following editing with compositions comprising Cas-CLOVER (5 μg) and gRNA pairs targeting KLKB1 (4 μg). On the y-axis, indels are shown as a percentage of modified reads of total sequencing reads. The x-axis shows the gRNA pairs that were tested (guide pairs #K1-#K17). B2M was used as a control.

FIG. 3 is a graph showing indels in HepaRG cells following editing with compositions comprising Cas-CLOVER (5 μg) and gRNA pairs targeting KLKB1 (4 μg). On the y-axis, indels are shown as a percentage of modified reads of total sequencing reads. The x-axis shows the gRNA pairs that were tested (Guide pairs #K1, #K4, #K5, #K15, and #K16).

FIG. 4 is a graph showing KLKB1 protein levels for each LNP concentration using a KLKB1 ELISA to determine the percent decrease in KLKB1 protein levels compared to levels in unedited baseline cells.

FIG. 5 is a schematic of an exemplary pharmaceutical study outlining the editing of murine KLKB1 locus in adult wild type mice.

FIG. 6 is a graph showing the editing efficiency of Cas-CLOVER in liver-humanized TK-Nog mice. The y-axis shows the percent of KLKB1 edited. The x-axis shows the Cas-CLOVER versions (CCv2 (Cas-CLOVER v2 dosed once), CCv2-x2 (Cas-CLOVER v2 dosed twice), and CCv3 (Cas-CLOVERv3 dosed once)) that were tested, compared to a PBS control.

FIG. 7 is a graph showing indels in mouse hepatoma cells following editing with compositions comprising Cas-CLOVER and gRNA pairs targeting KLKB1. On the y-axis, indels are shown as a percentage of modified reads of total sequencing reads. The x-axis shows the gRNA pairs that were tested (pair numbers 1-7).

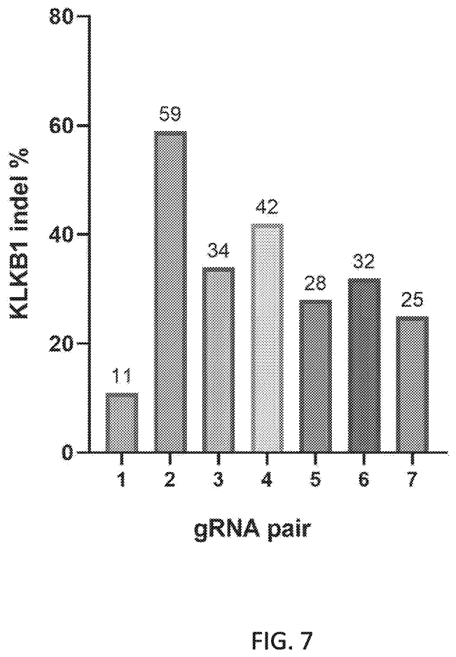

FIG. 8A is a graph showing the efficiency of KLKB1 editing with Cas-CLOVER in C57Bl/6 male mice. The y-axis shows the percent of KLKB1 edited. The x-axis shows the Cas-CLOVER versions (CCv2 1 mpk (Cas-CLOVER v2 dosed at 1 mg/kg), CCv3 1 mpk (Cas-CLOVERv3 dosed at 1 mg/kg), CCv2 2 mpk (Cas-CLOVER v2 dosed at 2 mg/kg) and CCv3 2 mpk (Cas-CLOVER v3 2 mg/kg)) that were tested compared to a PBS control. FIG. 8B is a graph showing the serum KLKB1 mRNA levels as a percent of baseline after treatment with Cas-CLOVER in C57Bl/6 male mice. The y-axis shows the serum KLKB1 mRNA levels as a percent of baseline. The x-axis shows the Cas-CLOVER versions (CCv2 1 mpk (Cas-CLOVER v2 dosed at 1 mg/kg), CCv3 1 mpk (Cas-CLOVER v3 dosed at 1 mg/kg), CCv2 2 mpk (Cas-CLOVER v2 dosed at 2 mg/kg) and CCv3 2 mpk (Cas-CLOVER v3 2 mg/kg)) that were tested compared to a PBS control.

FIG. 9A is a graph showing the efficiency of KLKB1 editing with Cas-CLOVER in C57B1/6 female mice. The y-axis shows the percent of KLKB1 edited. The x-axis shows the Cas-CLOVER versions (CCv2 1 mpk (Cas-CLOVER v2 dosed at 1 mg/kg), CCv3 1 mpk (Cas-CLOVER v3 dosed at 1 mg/kg), CCv2 2 mpk (Cas-CLOVER v2 dosed at 2 mg/kg) and CCv3 2 mpk (Cas-CLOVER v3 2 mg/kg)) that were tested compared to a PBS control. FIG. 9B is a graph showing the serum KLKB1 mRNA levels as a percent of baseline after treatment with Cas-CLOVER in C57Bl/6 female mice. The y-axis shows the serum KLKB1 mRNA levels as a percent of baseline. The x-axis shows the Cas-CLOVER versions (CCv2 1 mpk (Cas-CLOVER v2 dosed at 1 mg/kg), CCv3 1 mpk (Cas-CLOVER v3 dosed at 1 mg/kg), CCv2 2 mpk (Cas-CLOVER v2 dosed at 2 mg/kg) and CCv3 2 mpk (Cas-CLOVER v3 2 mg/kg)) that were tested compared to a PBS control.

FIG. 10 is a schematic diagram of the composition of the disclosure. A first fusion protein (e.g. Cas-Clover comprising a dCas9-linker-Clo051) is complexed with a first gRNA at the 5′ terminus of the genomic region. A second fusion protein (e.g. Cas-Clover comprising a dCas9-linker Clo051) is complexed with a second gRNA at the 3′ terminus of the genomic region. Targeting using gRNAs provides highly efficient and accurate targeting. Only when the Clo051 nucleases of the first fusion protein and the second fusion protein are brought in proximity, is a cut made to the genomic DNA template.

FIG. 11 is a schematic diagram depicting a partially humanized KLKB1 mouse model (huKLKB1 mice) used to evaluate the efficacy of P-KLKB1-101. The partially humanized mouse model was generated by germline introduction of the human sgRNA target sequences into the mKlkb1 gene to produce P-KLKB1-101-editable loci in all somatic and reproductive cells.

FIG. 12 is a graph illustrating the effects of increasing LNP dosage on Activated partial thromboplastin time (aPTT) in African green monkeys.

FIG. 13 is a graph illustrating the effects of increasing LNP dosage on the efficiency of KLKB1 editing with Cas-CLOVER in African green monkeys.

### DETAILED DESCRIPTION

The present invention provides compositions and methods for genetically modifying a genome to include a polynucleotide insertion, deletion and/or a substitution into chromosomal DNA that reduces the transcription of the KLKB1 gene. In particular, the present disclosure overcomes problems associated with current technologies by providing methods and compositions for efficiently genetically modifying cellular genomes to include polynucleotide insertions, deletions and/or substitutions, which is an advantageous validated target in the KLKB1 gene, a key mediator of the kallikrein bradykinin pathway that is hyperactivated in Hereditary Angioedema (HAE). These methods and compositions can be used for the treatment of Hereditary Angioedema Types I and II caused by mutations in C1-inh (SERPING1 gene). In some embodiments, the introduction of polynucleotide insertions, deletions and/or substitutions in the KLKB1 gene (or gene locus) of a cellular genome may result in reduced transcription of the KLKB1 gene and reduced KLKB1 protein expression, compared to cells not modified by a method or composition of the present disclosure. Patients with HAE suffer from spontaneous angioedema attacks 2 to 4 times per month. Without wishing to be bound by theory, reduced transcription of KLKB1 in patients with HAE may result in a reduction in one or more symptoms of HAE including reduced instances of spontaneous angioedema attacks which typically occur 2 to 4 times per month.

The genome editing systems of this disclosure can include two or more fusion proteins (e.g., Cas-Clover) and two or more gRNAs having a targeting domain that is complementary to a sequence in or near the target region. In certain embodiments, the two or more gRNAs are capable of targeting the fusion protein to the target region. In certain embodiments, the DNA binding region of KLKB1 is targeted for disruption. In certain embodiments, exons 6-12 of KLKB1 are targeted for disruption. In certain embodiments, the genome editing systems disclosed herein may be used to introduce a polynucleotide insertion, deletion, and/or substitution in the targeted region. The genome editing systems of the present disclosure can also be encapsulated in one or more Lipid Nanoparticles (LNPs). In certain embodiments, an LNP composition of the present disclosure can deliver a genome editing system of this disclosure to a target cell or tissue.

The present disclosure overcomes problems associated with current technologies by providing compositions comprising genetically engineered fusion molecules (e.g. Cas-Clover) for targeted reduction or elimination of gene products in a cell for use in in vivo gene therapy. The compositions comprising genetically engineered fusion molecules of the disclosure are useful for the treatment of genetic diseases. Non-limiting examples of genetic diseases include Hereditary Angioedema Types I and II. Accordingly, methods of making genetically engineered fusion molecules and pharmaceutical formulations thereof (e.g., lipid nanoparticle formulations) for use in in vivo delivery are also provided. As a non-limiting example, the magnitude of the improvement provided by the compositions the disclosure, could provide a key therapeutic threshold to reduce KLKB1 expression, which would provide therapeutic efficacy for treatment of Hereditary Angioedema Types I and II.

### Methods for Targeted Genome Editing at Selected Locus

### Gene Editing Compositions and Methods

The present disclosure provides a gene editing composition and/or a cell comprising the gene editing composition. The gene editing composition can comprise a sequence encoding a DNA binding domain and a sequence encoding a nuclease protein or a nuclease domain thereof. The sequence encoding a nuclease protein or the sequence encoding a nuclease domain thereof can comprise a nucleic acid sequence (e.g. DNA sequence, an RNA sequence, or a combination thereof). The nuclease or the nuclease domain thereof can comprise one or more of a CRISPR/Cas protein, a Transcription Activator-Like Effector Nuclease (TALEN), a Zinc Finger Nuclease (ZFN), and an endonuclease.

In some aspects, the nucleic acid molecule can be a synthetic nucleic acid molecule. In some aspects, the nucleic acid molecule can be a non-naturally occurring nucleic acid molecule. Modified nucleic acids can include, but are not limited to, 5-methoxy uridine (5moU), N1-methyl pseudouridine (me1ψ), pseudouridine (Y), 5-methylcytidine (5-MeC). In some aspects, the non-naturally occurring nucleic acid molecule can comprise at least one non-naturally occurring nucleotide. The at least one non-naturally occurring nucleotide can be any non-naturally occurring nucleotide known in the art. In some aspects, the nucleic acid molecule can be a modified nucleic acid molecule. In some aspects, the modified nucleic acid molecule can comprise at least one modified nucleotide. The at least one modified nucleotide can be any modified nucleic acid known in the art. In some aspects, the nucleic acid molecule may be a circular DNA molecule, such as, but not limited to, a DNA plasmid. In some aspects, the nucleic acid molecule can be a linearized DNA molecule, such as, but not limited to, a linearized DNA plasmid. In some aspect, the nucleic acid molecule can be a DoggyBone DNA molecule. In some aspects, the nucleic acid molecule can be a DNA nanoplasmid.

In some embodiments, a nucleic acid molecule of the present disclosure can be at least about 0.25 kb, or at least about 0.5 kb, or at least about 0.75 kb, or at least about 1.0 kb, or at least about 1.25 kb, or at least about 1.5 kb, or at least about 1.75 kb, or at least about 2.0 kb, or at least about 2.25 kb, or at least about 2.5 kb, or at least about 2.75 kb, or at least about 3.0 kb, or at least about 3.25 kb, or at least about 3.5 kb, or at least about 3.75 kb, or at least about 4.0 kb, or at least about 4.25 kb, or at least about 4.5 kb, or at least about 4.75 kb, or at least about 5.0 kb, or at least about 5.25 kb, or at least about 5.5 kb, or at least about 5.75 kb, or at least about 6.0 kb, or at least about 6.25 kb, or at least about 6.5 kb, or at least about 6.75 kb, or at least about 7.0 kb, or at least about 7.25 kb, or at least about 7.5 kb, or at least about 7.75 kb, or at least about 8.0 kb, or at least about 8.25 kb, or at least about 8.5 kb, or at least about 8.75 kb, or at least about 9.0 kb, or at least about 9.25 kb, or at least about 9.5 kb, or at least about 9.75 kb, or at least about 10.0 kb, or at least about 10.25 kb, or at least about 10.5 kb, or at least about 10.75 kb, or at least about 11.0 kb, or at least about 11.25 kb, or at least about 11.5 kb, or at least about 11.75 kb, or at least about 12 kb, or at least about 12.25 kb, or at least about 12.5 kb, or at least about 12.75 kb, or at least about 13.0 kb, or at least about 13.25 kb, or at least about 13.5 kb, or at least about 13.75 kb, or at least about 14.0 kb, or at least about 14.25 kb, or at least about 14.5 kb, or at least about 14.75 kb or at least about 15.0 kb in length.

A method for directing proteins to a specific locus in a genome of an organism is also disclosed herein. The method may comprise the steps of providing a DNA localization component and providing an effector molecule, wherein the DNA localization component and the effector molecule are capable of operatively linking via a non-covalent linkage.

Exemplary dCas9-Clo051 (Cas-CLOVER) Fusion Proteins

The nuclease or the nuclease domain thereof can comprise a nuclease-inactivated Cas (dCas) protein and an endonuclease. The endonuclease can comprise a Clo051 nuclease or a nuclease domain thereof. The gene editing composition can comprise a fusion protein comprising a mutated or nuclease-inactivated Cas9 (dCas9) protein and a Clo051 nuclease or a Clo051 nuclease domain. The gene editing composition can further comprise a guide sequence. The guide sequence comprises an RNA sequence.

The disclosure provides compositions comprising a Cas9 operatively linked to an effector. The disclosure provides a fusion protein comprising, consisting essentially of or consisting of a DNA localization component and an effector molecule, wherein the effector comprises a Cas9. A Cas9 of the disclosure can comprise an effector comprising a type IIS endonuclease.

The disclosure provides compositions comprising an inactivated, Cas9 (dSaCas9) operatively linked to an effector. The disclosure provides a fusion protein comprising, consisting essentially of or consisting of a DNA localization component and an effector molecule, wherein the effector comprises an inactivated Cas9 (dSaCas9). An inactivated Cas9 (dSaCas9) construct of the disclosure can comprise an effector comprising a type IIS endonuclease. A dSaCas9 can comprise the amino acid sequence of SEQ ID NO: 40, which includes a D10A and an N580A mutation relative to wildtype Cas9 to inactivate the catalytic site.

The disclosure provides compositions comprising an inactivated Cas9 (dCas9) operatively linked to an effector. The disclosure provides a fusion protein comprising, consisting essentially of, or consisting of, a DNA localization component and an effector molecule, wherein the effector comprises an inactivated Cas9 (dCas9).

The dCas9 can be isolated or derived from Streptococcus pyogenes. The dCas9 can comprise a dCas9 with substitutions at amino acid positions 10 and 840 of the wildtype sequence, which inactivate the catalytic site. In some aspects, these substitutions are D10A and H840A. The dCas9 can comprise the amino acid sequence of SEQ ID NO: 41 or SEQ ID NO: 42. In some aspects, the C-terminus of the dCas9 is joined to N-terminus of the Clo051 polypeptide or nuclease domain thereof via the peptide linker sequence of GGGGS (SEQ ID NO: 57).

In some embodiments, the C-terminus of the dCas9 or inactivated nuclease domain thereof is joined to the N-terminus of the Clo051 polypeptide or nuclease domain thereof via peptide linker sequence selected from GGGGS (SEQ ID NO: 57).

An exemplary Clo051 nuclease domain comprises, consists essentially of or consists of, the amino acid sequence of SEQ ID NO: 43. In some aspects, the Clo051 nuclease domain comprises at least one amino acid substitution relative to SEQ ID NO: 43. In some aspects, the amino acid substitution is in the alpha-helix-loop domain of the Clo051 nuclease. In some aspects, the amino acid substitution is at position 35, 37, 60, 92, 98, 100 or 146 of SEQ ID NO: 43. In some aspects, the amino acid substitution is at position 37 of SEQ ID NO: 43. In some aspects, the amino acid substitution is at positions 37 and 92 of SEQ ID NO: 43.

An exemplary dCas9-Clo051 (Cas-CLOVER) fusion protein can comprise, consist essentially of, or consist of, the amino acid sequence of SEQ ID NO: 44. The exemplary dCas9-Clo051 fusion protein can be encoded by a polynucleotide which comprises, consists essentially of, or consists of, the nucleic acid sequence of SEQ ID NO: 45. The nucleic acid encoding the dCas9-Clo051 fusion protein can be DNA or RNA.

An exemplary dCas9-Clo051 (Cas-CLOVER) fusion protein can comprise, consist essentially of, or consist of, the amino acid sequence of SEQ ID NO: 46. The exemplary dCas9-Clo051 fusion protein can be encoded by a polynucleotide which comprises, consists essentially of, or consists of, the nucleic acid sequence of SEQ ID NO: 47. The nucleic acid encoding the dCas9-Clo051 fusion protein can be DNA or RNA.

An exemplary dCas9-Clo051 fusion (Cas-CLOVER) fusion protein of the disclosure may further comprise at least one nuclear localization sequence (NLS). In some embodiments, the dCas9-Clo051 fusion protein of the disclosure comprises at least two nuclear localization sequences. In some embodiments, the NLS is located on the N-terminal end of the dCas9-Clo051 fusion protein (NLS-dCas9-Clo051). In some embodiments, the NLS is located on the C-terminal end of the dCas9-Clo051 fusion protein (dCas9-Clo051-NLS). In some embodiments, the NLS is located on the N-terminal end and at the C-terminal end of the dCas9-Clo051 fusion protein (“NLS-dCas9-Clo051-NLS” or “wildtype Cas-CLOVER” or “dspCas9 Ca-CLOVER”).

The NLS-dCas9-Clo051-NLS (“wildtype Cas-CLOVER”, or “Cas-CLOVER v2”, or “CCv2”, or “dspCas9 Cas-CLOVER”) fusion protein can comprise, consist essentially of, or consist of, the amino acid sequence of SEQ ID NO: 35.

Cas-CLOVER v2 amino acid sequence (NLS amino acid
sequence is bolded and underlined; linker is
bolded and italicized)
(SEQ ID NO: 35)
MAPKKKRKVEGIKSNISLLKDELRGQISHISHEYLSLIDLAFDSKQNRL
FEMKVLELLVNEYGFKGRHLGGSRKPDGIVYSTTLEDNFGIIVDTKAYS
EGYSLPISQADEMERYVRENSNRDEEVNPNKWWENFSEEVKKYYFVFIS
GSFKGKFEEQLRRLSMTTGVNGSAVNVVNLLLGAEKIRSGEMTIEELER
AMFNNSEFILKYGGGGSDKKYSIGLAIGTNSVGWAVITDEYKVPSKKFK
VLGNIDRHSIKKNLIGALLFDSGETAEATRLKRTARRRYTRRKNRICYL
QEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSD
VDKLFIQLVQTYNQLFEENPINASGVDAKAILSARLSKSRRLENLIAQL
PGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDE
HHQDLTLLKALVRQQLPEKYKEIFFDQSKNGYAGYIDGGASQEEFYKFI
KPILEKMDGTEELLVKLNREDLLRKQRTFDNGSIPHQIHLGELHAILRR
QEDFYPFLKDNREKIEKILTFRIPYYVGPLARGNSRFAWMIRKSEETIT
PWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKI
ECFDSVEISGVEDRFNASLGTYHDLLKIIKDKDFLDNEENEDILEDIVL
TLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQ
TTQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAG
FIKRQLVETRQITKHVAQILDSRMNTKYDENDKLIREVKVITLKSKLVS
DFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPL
IETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENG
RKRMLASAGELQKGNELALPSKYVNFLYLASHYEKLKGSPEDNEQKQLF
VEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLY
KKRKVSS.ETRIDLSQLGGDGSPK

The nucleic acid encoding the NLS-dCas9-Clo051-NLS (“wildtype Cas-CLOVER”, or “Cas-CLOVER v2”, or “CCv2”, or “dspCas9 Cas-CLOVER”) fusion protein can be DNA or RNA. In some embodiments, a dCas9-Clo051 fusion protein comprising two NLS regions is encoded by an mRNA sequence comprising, consisting essentially of or consisting of SEQ ID NO: 36 or a DNA sequence comprising, consisting essentially of or consisting of SEQ ID NO: 37.

NLS-dCas9-Clo051-NLS mRNA sequence
(SEQ ID NO: 36)
auggcucccaagaagaagcggaaggucGAGGGCAUCAAGAGCAACAUCAGCCUGCUGAAGGACGAG
CUGAGAGGCCAGAUCAGCCACAUCUCCCACGAGUACCUGAGCCUGAUCGACCUGGCCUUCGACCCC
AAGCAGAACCGGCUGUUCGAGAUGAAGGUGCUGGAACUGCUGGUCAACGAGUACGGCUUCAAGGGC
AGACACCUCGGCGGCAGCAGAAAGCCUGAUGGCAUCGUGUACAGCACCACACUCGAGGACAACUUC
GGCAUCAUCGUGGACACCAAGGCCUACAGCGAGGGCUACAGCCUGCCUAUCUCUCAGGCCGACGAG
AUGGAAAGAUACGUGCGCGAGAACAGCAACCGCGACGAGGAAGUGAACCCCAACAAGUGGUGGGAG
AACUUCAGCGAGGAAGUCAAAAAGUACUACUUCGUGUUCAUCAGCGGCAGCUUUAAGGGCAAGUUC
GAGGAACAGCUGCGGCGGCUGUCUAUGACCACAGGCGUUAACGGCAGCGCCGUGAACGUGGUCAAU
CUGCUGCUGGGCGCCGAGAAGAUUAGAAGCGGCGAGAUGACCAUCGAGGAACUGGAACGGGCCAUG
UUCAACAACAGCGAGUUCAUCCUGAAGUACggcggaggcggcagcgacaagaaguacucuaucgga
cuggccaucggcaccaacucuguuggaugggccgugaucaccgacgaguacaaggugcccagcaag
aaauucaaagugcugggcaacaccgaccggcacagcaucaagaagaaucugaucggcgcccugcug
uucgacucuggcgaaacagccgaagccaccagacugaagagaaccgccagacggcgguacaccaga
agaaagaaccggaucugcuaccugcaagagaucuucagcaacgagauggccaagguggacgacagc
uucuuccacagacuggaagaguccuuccugguggaagaggacaagaagcacgagcggcaccccauc
uucggaaauaucguggacgagguggccuaccacgagaaguaccccaccaucuaccaccugagaaag
aaacugguggacagcaccgacaaggccgaccugcgacugaucuaucuggcccuggcucacaugauc
aaguuccggggccacuuccugaucgagggcgaccugaauccugacaacuccgacguggacaagcug
uucauccagcuggugcagaccuacaaucagcuguucgaagagaaucccaucaacgccucuggcgug
gacgccaaagccauccugucugccagacugagcaagagcagacggcuggaaaaccugaucgcucag
cugcccggcgagaagaagaauggccuguucggcaaccugauugcccugucucugggccugacaccu
aacuucaaguccaacuucgaucuggccgaggaugccaaacugcagcuguccaaggacaccuacgac
gacgaccuggauaaccugcuggcccagaucggcgaucaguacgccgacuuguuucuggccgccaag
aaccugucugacgccauccugcugagcgacauccugagagugaacaccgagaucacaaaggccccu
cugagcgccucuaugaucaagagauacgacgagcaccaccaggaucugacccugcugaaagcucuc
gucaggcagcagcugccagagaaguacaaagagauuuucuucgaccagagcaagaacggcuacgcc
ggcuacauugauggcggagccagccaagaggaauucuacaaguucaucaagcccauccucgagaag
auggacggcacagaggaacugcucgugaagcugaacagagaggaccugcugcggaagcagcggacc
uucgacaauggcucuaucccucaccagauccaccugggagagcugcacgccauucugcggagacaa
gaggacuuuuacccauuccugaaggacaaccgggaaaagauugagaagauccugaccuucaggauc
cccuacuacgugggaccacuggccagaggcaauagcagauucgccuggaugaccagaaagagcgag
gaaaccaucacacccuggaacuucgaagaggugguggacaagggcgccagcgcucaguccuucauc
gagcggaugaccaauuucgacaagaaucugcccaacgagaaagugcugcccaagcacucccugcug
uacgaguacuucaccguguacaacgagcugaccaaagugaaauacgugaccgagggaaugagaaag
cccgccuuucuguccggcgagcagaaaaaggccaucguggaucugcuguucaagaccaaccggaaa
gugaccgugaagcagcugaaagaggacuacuucaagaaaaucgagugcuucgacuccguggaaauc
agcggcguggaagaucgguucaaugccagccugggcacauaccacgaucugcugaaaauuaucaag
gacaaggacuuccuggacaacgaggaaaacgaggacauccuugaggacaucgugcugacccugaca
cuguucgaggacagagagaugaucgaggaaaggcugaaaacauacgcccaccuguucgacgacaaa
gucaugaagcaacugaagcggcggcgcuacacaggcuggggcagacugucuagaaagcugaucaac
ggcauccgggacaagcaguccggcaagaccauccuggacuuucugaaguccgacggcuucgccaac
agaaacuucaugcagcugauucacgacgacagccucaccuucaaagaggacauucagaaggcccag
guuuccggccagggcgauucucugcacgagcacauugccaaucuggccggcucucccgccauuaag
aagggcauucugcagacagugaaagugguggaugagcuggucaaagugauggggagacacaagccc
gagaacaucgugaucgaaauggccagagagaaccagaccacacagaagggccagaagaacucccgc
gagagaaugaagcggaucgaagagggaaucaaagagcuggggagccagauccugaaagaacacccc
guggaaaacacccagcugcagaacgagaagcuguaccuguacuaccuccagaacggccgggauaug
uacguggaccaagagcuggacaucaaccgccugagcgacuacgauguggacgcuaucgugccccag
ucuuuucugaaagaugacuccaucgacaacaaggugcugaccagaagcgauaagaaccggggcaag
agcgacaacgugcccucugaagaggucgugaagaagaugaagaacuacuggcgacagcugcugaac
gccaagcugauuacccagcggaaguucgauaaccugaccaaggccgagagaggcggccugucugaa
cuggauaaggccggcuucaucaagagacagcugguggaaacccggcagaucaccaaacacguggca
cagauucuggacucccggaugaacaccaaquacgaugagaacgacaaacugauccgggaagugaaa
gucaucacccugaaguccaagcugguguccgauuuccggaaggauuuccaguucuacaaagugcgg
gaaaucaacaacuaccaucacgcccacgacgccuaccugaaugccguuguuggaacagcccugauc
aagaaguaucccaagcuggaaagcgaguucguguacggcgacuacaagguguacgacgugcggaag
augaucgccaagagcgagcaagagauuggaaaggcuaccgccaaauacuucuucuacuccaacauc
augaacuuuuucaagacagagaucacccucgccaacggcgagaucagaaagcggccucugaucgag
acaaacggcgaaaccggcgagauugugugggauaagggcagagacuuugccacagugcggaaggug
cucagcaugccccaagugaauaucgugaaaaagaccgaggugcagacaggcggcuucagcaaagag
uccauucugccuaagcggaacuccgacaagcugaucgcccggaagaaggacugggaccccaagaaa
uacggcggcuucgauagcccuaccguggccuauucugugcuggugguggccaaaguggaaaaggga
aaguccaagaagcucaagagcgucaaagaacuccugggcaucaccaucauggaacgguccagcuuc
gagaagaacccuaucgacuuucuggaagccaagggcuacaaagaagucaagaaggaccugaucauc
aagcuccccaaguacagccuguucgagcuggaaaauggccggaagcggaugcuggcuucugcuggc
gaacugcagaagggaaacgaacuggcccugccuagcaaauaugugaacuuccuguaccuggccagc
cacuaugagaagcugaagggcagccccgaggacaaugagcagaagcagcuuuucgucgagcagcac
aagcacuaccuggacgagaucaucgagcagaucuccgaguucuccaagagagugauccuggccgac
gccaaccuggacaagguucuguccgccuacaacaagcaccgggauaagcccaucagagagcaggcc
gagaauaucauccaccuguuuacccugaccaaccugggagccccugccgccuucaaguacuucgac
accaccaucgaccggaagcgcuacaccagcaccaaagaagugcuggacgccacacugauccaccag
agcaucaccggccuguacgagacacggaucgaucugucucagcuuggaggcgacggcagcccuaag
aagaagagaaagguuuccagcuaauaa.

A cell comprising the gene editing composition can express the gene editing composition stably or transiently.

### Exemplary Mutant Cas-CLOVER Fusion Proteins

In some aspects, NLS-dCas9-Clo051-NLS (“wildtype Cas-CLOVER”) comprises at least one amino acid substitution. In some aspects, the amino acid substitution is located in the Clo051 domain of the NLS-dCas9-Clo051-NLS.

In some aspects, the NLS-dCas9-Clo051-NLS of SEQ ID NO: 35 can comprise at least one substitution at amino acid positions 42, 44, 67, 105, 107 or 153. In some aspects, the amino acid substitutions are F42E, F42D, S44E, S44P, R67E, I105Q, Q107A, Q107E, Q107H, Q107D and/or K153D. In some aspects, the amino acid substitution is S44P.

An exemplary S44P mutant NLS-dCas9-Clo051-NLS (“$44P Cas-CLOVER”, or “S44P CC”, or “S44P”, or “Cas-CLOVERv3”, or “CCv3”) fusion protein can comprise, consist essentially of, or consist of, the amino acid sequence of SEQ ID NO: 38.

Cas-CLOVER v3 amino acid sequence (NLS amino
acid sequence is bolded and underlined;
linker is bolded and italicized)
(SEQ ID NO: 38)
MAPKKKRKVEGIKSNISLLKDELRGQISHISHEYLSLIDLAFDPKQNRL
FEMKVLELLVNEYGFKGRHLGGSRKPDGIVYSTTLEDNFGIIVDTKAYS
EGYSLPISQADEMERYVRENSNRDEEVNPNKWWENFSEEVKKYYFVFIS
GSFKGKFEEQLRRLSMTTGVNGSAVNVVNLLLGAEKIRSGEMTIEELER
AMFNNSEFILKYGGGGSDKKYSIGLAIGTNSVGWAVITDEYKVPSKKFK
VLGNTDRHSIKKNLIGALLFDSGETAEATRLKRTARRRYTRRKNRICYL
QEIFSNEMAKVDDSFFHRLEESFLVEEDKKHERHPIFGNIVDEVAYHEK
YPTIYHLRKKLVDSTDKADLRLIYLALAHMIKFRGHFLIEGDLNPDNSD
VDKLFIQLVQTYNQLFEENPINASGVDAKAILSARLSKSRRLENLIAQL
PGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDE
HHQDLILLKALVRQQLPEKYKEIFFDQSKNGYAGYIDGGASQEEFYKFI
KPILEKMDGTEELLVKLNREDLLRKQRTFDNGSIPHQIHLGELHAILRR
QEDFYPFLKDNREKIEKILTFRIPYYVGPLARGNSRFAWMTRKSEETIT
PWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKI
ECEDSVEISGVEDRFNASLGTYHDLLKIIKDKDFLDNEENEDILEDIVL
TLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDS
LHEHIANLAGSPAIKKGILQTVKVVDELVKVMGRHKPENIVIEMARENQ
TTQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRGK
SDNVPSEEVVKKMKNYWRQLLNAKLITQRKFDNLTKAERGGLSELDKAG
FIKRQLVETRQITKHVAQILDSRMNTKYDENDKLIREVKVITLKSKLVS
DFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPL
IETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSV
KELLGITIMERSSFEKNPIDFLEAKGYKEVKKDLIIKLPKYSLFELENG
RKRMLASAGELQKGNELALPSKYVNFLYLASHYEKLKGSPEDNEQKQLF
VEQHKHYLDEIIEQISEFSKRVILADANLDKVLSAYNKHRDKPIREQAE
NIIHLFTLTNLGAPAAFKYFDTTIDRKRYTSTKEVLDATLIHQSITGLY
ETRIDLSQLGGDGSPKKKRKVSS

The Cas-CLOVER v3 fusion protein can be encoded by a polynucleotide which comprises, consists essentially of, or consists of, the nucleic acid sequence of SEQ ID NO: 39. The nucleic acid encoding the dCas9-Clo051 fusion protein can be DNA or RNA.

Cas-CLOVER v3 mRNA sequence
(SEQ ID NO: 39)
acauuugcuucugacacaacuguguucacuagcaaccucaaacagacaccaagcuugccaccauggcucccaagaagaag
cggaaggucGAGGGCAUCAAGAGCAACAUCAGCCUGCUGAAGGACGAGCUGAGAGGCCAGAUCAGCCACAUCUCCCACGA
GUACCUGAGCCUGAUCGACCUGGCCUUCGACCCCAAGCAGAACCGGCUGUUCGAGAUGAAGGUGCUGGAACUGCUGGUCA
ACGAGUACGGCUUCAAGGGCAGACACCUCGGCGGCAGCAGAAAGCCUGAUGGCAUCGUGUACAGCACCACACUCGAGGAC
AACUUCGGCAUCAUCGUGGACACCAAGGCCUACAGCGAGGGCUACAGCCUGCCUAUCUCUCAGGCCGACGAGAUGGAAAG
AUACGUGCGCGAGAACAGCAACCGCGACGAGGAAGUGAACCCCAACAAGUGGUGGGAGAACUUCAGCGAGGAAGUCAAAA
AGUACUACUUCGUGUUCAUCAGCGGCAGCUUUAAGGGCAAGUUCGAGGAACAGCUGCGGCGGCUGUCUAUGACCACAGGC
GUUAACGGCAGCGCCGUGAACGUGGUCAAUCUGCUGCUGGGCGCCGAGAAGAUUAGAAGCGGCGAGAUGACCAUCGAGGA
ACUGGAACGGGCCAUGUUCAACAACAGCGAGUUCAUCCUGAAGUACggcggaggcggcagcgacaagaaguacucuaucg
gacuggccaucggcaccaacucuguuggaugggccgugaucaccgacgaguacaaggugcccagcaagaaauucaaagug
cugggcaacaccgaccggcacagcaucaagaagaaucugaucggcgcccugcuguucgacucuggcgaaacagccgaagc
caccagacugaagagaaccgccagacggcgguacaccagaagaaagaaccggaucugcuaccugcaagagaucuucagca
acgagauggccaagguggacgacagcuucuuccacagacuggaagaguccuuccugguggaagaggacaagaagcacgag
cggcaccccaucuucggaaauaucguggacgagguggccuaccacgagaaguaccccaccaucuaccaccugagaaagaa
acugguggacagcaccgacaaggccgaccugcgacugaucuaucuggcccuggcucacaugaucaaguuccggggccacu
uccugaucgagggcgaccugaauccugacaacuccgacguggacaagcuguucauccagcuggugcagaccuacaaucag
cuguucgaagagaaucccaucaacgccucuggcguggacgccaaagccauccugucugccagacugagcaagagcagacg
gcuggaaaaccugaucgcucagcugcccggcgagaagaagaauggccuguucggcaaccugauugcccugucucugggcc
ugacaccuaacuucaaguccaacuucgaucuggccgaggaugccaaacugcagcuguccaaggacaccuacgacgacgac
cuggauaaccugcuggcccagaucggcgaucaguacgccgacuuguuucuggccgccaagaaccugucugacgccauccu
gcugagcgacauccugagagugaacaccgagaucacaaaggccccucugagcgccucuaugaucaagagauacgacgagc
accaccaggaucugacccugcugaaagcucucgucaggcagcagcugccagagaaguacaaagagauuuucuucgaccag
agcaagaacggcuacgccggcuacauugauggcggagccagccaagaggaauucuacaaguucaucaagcccauccucga
gaagauggacggcacagaggaacugcucgugaagcugaacagagaggaccugcugcggaagcagcggaccuucgacaaug
gcucuaucccucaccagauccaccugggagagcugcacgccauucugcggagacaagaggacuuuuacccauuccugaag
gacaaccgggaaaagauugagaagauccugaccuucaggauccccuacuacgugggaccacuggccagaggcaauagcag
auucgccuggaugaccagaaagagcgaggaaaccaucacacccuggaacuucgaagaggugguggacaagggcgccagcg
cucaguccuucaucgagcggaugaccaauuucgacaagaaucugcccaacgagaaagugcugcccaagcacucccugcug
uacgaguacuucaccguguacaacgagcugaccaaagugaaquacgugaccgagggaaugagaaagcccgccuuucuguc
cggcgagcagaaaaaggccaucguggaucugcuguucaagaccaaccggaaagugaccgugaagcagcugaaagaggacu
acuucaagaaaaucgagugcuucgacuccguggaaaucagcggcguggaagaucgguucaaugccagccugggcacauac
cacgaucugcugaaaauuaucaaggacaaggacuuccuggacaacgaggaaaacgaggacauccuugaggacaucgugcu
gacccugacacuguucgaggacagagagaugaucgaggaaaggcugaaaacauacgcccaccuguucgacgacaaaguca
ugaagcaacugaagcggcggcgcuacacaggcuggggcagacugucuagaaagcugaucaacggcauccgggacaagcag
uccggcaagaccauccuggacuuucugaaguccgacggcuucgccaacagaaacuucaugcagcugauucacgacgacag
ccucaccuucaaagaggacauucagaaggcccagguuuccggccagggcgauucucugcacgagcacauugccaaucugg
ccggcucucccgccauuaagaagggcauucugcagacagugaaagugguggaugagcuggucaaagugauggggagacac
aagcccgagaacaucgugaucgaaauggccagagagaaccagaccacacagaagggccagaagaacucccgcgagagaau
gaagcggaucgaagagggaaucaaagagcuggggagccagauccugaaagaacaccccguggaaaacacccagcugcaga
acgagaagcuguaccuguacuaccuccagaacggccgggauauguacguggaccaagagcuggacaucaaccgccugagc
gacuacgauguggacgcuaucgugccccagucuuuucugaaagaugacuccaucgacaacaaggugcugaccagaagcga
uaagaaccggggcaagagcgacaacgugcccucugaagaggucgugaagaagaugaagaacuacuggcgacagcugcuga
acgccaagcugauuacccagcggaaguucgauaaccugaccaaggccgagagaggcggccugucugaacuggauaaggcc
ggcuucaucaagagacagcugguggaaacccggcagaucaccaaacacguggcacagauucuggacucccggaugaacac
caaauacgaugagaacgacaaacugauccgggaagugaaagucaucacccugaaguccaagcugguguccgauuuccgga
aggauuuccaguucuacaaagugcgggaaaucaacaacuaccaucacgcccacgacgccuaccugaaugccguuguugga
acagcccugaucaagaaguaucccaagcuggaaagcgaguucguguacggcgacuacaagguguacgacgugcggaagau
gaucgccaagagcgagcaagagauuggaaaggcuaccgccaaauacuucuucuacuccaacaucaugaacuuuuucaaga
cagagaucacccucgccaacggcgagaucagaaagcggccucugaucgagacaaacggcgaaaccggcgagauugugugg
gauaagggcagagacuuugccacagugcggaaggugcucagcaugccccaagugaauaucgugaaaaagaccgaggugca
gacaggcggcuucagcaaagaguccauucugccuaagcggaacuccgacaagcugaucgcccggaagaaggacugggacc
ccaagaaauacggcggcuucgauagcccuaccguggccuauucugugcuggugguggccaaaguggaaaagggaaagucc
aagaagcucaagagcgucaaagaacuccugggcaucaccaucauggaacgguccagcuucgagaagaacccuaucgacuu
ucuggaagccaagggcuacaaagaagucaagaaggaccugaucaucaagcuccccaaguacagccuguucgagcuggaaa
auggccggaagcggaugcuggcuucugcuggcgaacugcagaagggaaacgaacuggcccugccuagcaaauaugugaac
uuccuguaccuggccagccacuaugagaagcugaagggcagccccgaggacaaugagcagaagcagcuuuucgucgagca
gcacaagcacuaccuggacgagaucaucgagcagaucuccgaguucuccaagagagugauccuggccgacgccaaccugg
acaagguucuguccgccuacaacaagcaccgggauaagcccaucagagagcaggccgagaauaucauccaccuguuuacc
cugaccaaccugggagccccugccgccuucaaguacuucgacaccaccaucgaccggaagcgcuacaccagcaccaaaga
agugcuggacgccacacugauccaccagagcaucaccggccuguacgagacacggaucgaucugucucagcuuggaggcg
acggcagcccuaagaagaagagaaagguuuccagcuaauaaggcggccgcccucgccccggaccugcccucccgccaggu
gcacccaccugcaauaaaugcagcgaagccgggagaauucccucgccccggaccugcccucccgccaggugcacccaccu
gcaauaaaugcagcgaagccgggagcggccgcggauccccggguaccgaauucgauaucucuauagugucaccuaaauuu
aauuaaaaaaaaaaaaaaaaaaaaaaaaaaaaaaaaaaaaaaaaaaaaaaaaaaaaaaaaaaaaaaaaaaaaaaaaaaaa
aaaA

gRNAS

As used herein, the term “guide sequence” or “spacer” in the context of a Cas-Clover system or a CRISPR-Cas9 system, comprises any polynucleotide molecule having sufficient complementarity with a target nucleic acid sequence to hybridize with the target nucleic acid sequence and direct sequence-specific binding of a nucleic acid-targeting complex to the target nucleic acid sequence. The guide sequence may comprise both RNA and DNA polynucleotides. The guide sequence may form a duplex with a target sequence. The duplex may be a DNA duplex, an RNA duplex, or an RNA/DNA duplex. The terms “guide molecule”, “guide RNA”, “gRNA”, “single guide RNA” and “sgRNA” are used interchangeably herein to refer to RNA-based molecules that are capable of forming a complex with a Cas-Clover or a CRISPR-Cas protein and comprises a guide sequence having sufficient complementarity with a target nucleic acid sequence to hybridize with the target nucleic acid sequence and direct sequence-specific binding of the complex to the target nucleic acid sequence. The guide molecule or guide RNA may encompass RNA-based molecules having one or more chemical modifications (e.g., by chemically linking two ribonucleotides or by replacement of one or more ribonucleotides with one or more deoxyribonucleotides), as described herein. The guide sequence may also partially comprise RNA and DNA-based nucleotides in which the molecule is chimeric for RNA and DNA nucleobases (e.g., containing either ribose or deoxyribose sugars).

The term “target region”, “target sequence” or “protospacer” as used interchangeably herein refers to the region of the target gene or genomic target site, to which the Cas-Clover system or the CRISPR/Cas9-based system targets. The Cas-Clover or the CRISPR/Cas9-based system may include at least two gRNAs, wherein the gRNAs target different DNA sequences. The target DNA sequences may be overlapping. The Cas-Clover system may include at least two gRNAs, wherein the gRNAs target different DNA sequences. The target sequence or protospacer is generally followed by a protospacer adjacent motif (PAM) sequence at the 3′ end of the protospacer. Different Type II CRISPR systems have differing PAM requirements. For example, the Streptococcus pyogenes Type II system uses an “NGG” sequence, where “N” can be any nucleotide.

The guide RNA or the guide RNA of a Cas-Clover protein or a CRISPR-Cas protein may comprise a tracr-mate sequence (encompassing a “direct repeat” in the context of an endogenous CRISPR system) and a guide sequence (also referred to as a “spacer” in the context of an endogenous CRISPR system). In some embodiments, the Cas-Clover or the CRISPR-Cas system or complex as described herein does not comprise and/or does not rely on the presence of a tracr sequence. In certain embodiments, the guide molecule may comprise, consist essentially of, or consist of a direct repeat sequence fused or linked to a guide sequence or spacer sequence.

In some embodiments, the guide RNA comprises a guide sequence and a scaffold sequence. In some embodiments, the scaffold sequence is isolated from Streptococcus pyogenes. In some embodiments, the Streptococcus pyogenes scaffold sequence comprises the nucleic acid sequence:

(SEQ ID NO: 48)
GUUUUAGAGCUAGAAAUAGCAAGUUAAAAUAAGGCUAGUCCGUUAUCAA
CUUGAAAAAGUGGCACCGAGUCGGUGCUUUU.

In some embodiments, the scaffold sequence is isolated from Staphylococcus aureus. In some embodiments, the Staphylococcus aureus scaffold sequence comprises the nucleic acid sequence:

(SEQ ID NO: 58)
GUUUUAGUACUCUGGAAACAGAAUCUACUAAAACAAGGCAAAAUGCCGU
GUUUAUCUCGUCAACUUGUUGGCGAGAUUUU.

In certain embodiments, the guide sequence or spacer of the guide molecules is 15 to 50 nucleotides in length. In certain embodiments, the spacer of the guide RNA is at least 15 nucleotides in length. In certain embodiments, the spacer is from 15 to 17 nucleotides in length, from 17 to 20 nucleotides in length, from 20 to 24 nucleotides in length, from 23 to 25 nucleotides in length, from 24 to 27 nucleotides in length, from 27 to 30 nucleotides in length, from 30 to 35 nucleotides in length, or greater than 35 nucleotides in length.

In some embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 nucleotides in length.

In some embodiments, the sequence of the guide molecule (direct repeat and/or spacer) is selected to reduce the degree of secondary structure within the guide molecule. In some embodiments, about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and P A Carr and G M Church, 2009, Nature Biotechnology 27 (12): 1151-62).

As described herein, the Cas-Clover system and the CRISPR/Cas9 system utilizes one or more targeting gRNAs that provides the targeting of the Cas-Clover system and the CRISPR/Cas9-based system. The gRNA may be a fusion of two noncoding RNAs: a crRNA and a tracrRNA. The sgRNA may target any desired DNA sequence by exchanging the sequence encoding a 20 bp protospacer which confers targeting specificity through complementary base pairing with the desired DNA target. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to cleave the target nucleic acid.

In some embodiments, the gRNA targets a KLKB1 gene locus (e.g., the KLKB1 open reading frame (ORF) or 0-1000 bp upstream of the open reading frame). In some embodiments, the gRNA targets a sequence near a KLKB1 gene locus. In some embodiments, the gRNA targets a region 0-50 bp, 0-100 bp, 0-150 bp, 0-200 bp, or 0-250 bp upstream or downstream of the KLKB1 gene ORF. In some embodiments, the gRNA targets a region 0-50 bp, 0-100 bp, 0-150 bp, 0-200 bp, 0-250 bp, 0-300 bp, 0-350 bp, 0-400 bp, 0-450 bp, 0-500 bp, 0-550 bp, 0-600 bp, 0-650 bp, 0-700 bp, 0-750 bp, 0-800 bp, 0-850 bp, 0-900 bp, 0-950 bp or 0-1000 bp upstream of the transcription start site of the KLKB1 gene. In some embodiments, the gRNA targets a region within about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp or about 1500 bp upstream of the KLKB1 ORF.

A gRNA can be divided into a target binding region (also referred to herein as a “targeting sequence”) and a Cas9 binding region. The target binding region hybridizes with a target region in a target gene or intergenic region. Methods for designing such target binding regions are known in the art, see, e.g., Doench et al., Nat Biotechnol. (2014) 32:1262-7; and Doench et al., Nat Biotechnol. (2016) 34:184-91, incorporated by reference herein in their entirety. Design tools are available at, e.g., Feng Zhang lab's target Finder, Michael Boutros lab's Target Finder (E-CRISP), RGEN Tools (Cas-OF Finder), CasFinder, and CRISPR Optimal Target Finder. In certain embodiments, the target binding region can be between about 15 and about 50 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 nucleotides in length). In certain embodiments, the target binding region can be between about 19 and about 21 nucleotides in length. In one embodiment, the target binding region is 15, 16, 17, 18, 19, 20, 21, 22, 23, 24, or 25 nucleotides in length.

In one embodiment, the target binding region is complementary, e.g., completely complementary, to the target region in the target gene. In one embodiment, the target binding region is substantially complementary to the target region in the target gene. In one embodiment, the target binding region comprises no more than 1, 2, 3, 4, 5, 6, 7, 8, 9, or 10 nucleotides that are not complementary to the target region in the target gene.

Exemplary gRNAs of the disclosure include but are not limited to sequences for targeting KLKB1 gene locus.

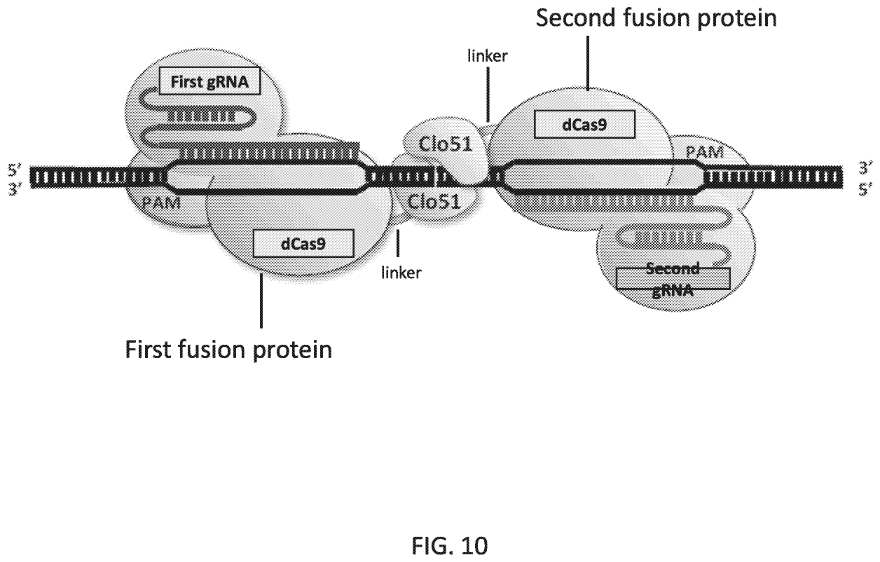

In some embodiments, the first gRNA (also referred to as “left gRNA”) binds to a template sequence at the 5′ terminus of the target gene locus and the second gRNA (also referred to as “right gRNA”) binds to a template sequence at the 3′ terminus of the target gene locus. A schematic diagram is shown in FIG. 10.

Exemplary gRNAs of the disclosure comprise, consist essentially of or consists of the target sequences as shown in Table 1 and Table 2. In some embodiments, a KLKB1 gene gRNA sequence of Table 1 comprises the following modifications: 2′-O-methyl analogs (OMe) on the first three bases and last three positions, and 3′ phosphorothioate internucleotide linkages (PS) between the first three bases and between the last two bases. In some embodiments, each sequence of Table 2 comprises the following modifications: each uridine residue in the mRNA is N1-methylpsuedouridine.

TABLE 1
Exemplary gRNAs of the disclosure targeting human KLKB1 gene

| Pair | | SEQ | | SEQ | Conserved |
|---|---|---|---|---|---|
| # | First/left gRNA | ID NO: | Second/right gRNA | ID NO: | in cyno |
| K1 | TGCCATTCTAAAATTT | SEQ ID | AACACAAAAAGATATC | SEQ ID | No |
| | ACCC | NO: 1 | AGAT | NO: 18 | |
| K2 | ACAAGCACACGCATTG | SEQ ID | AAAGAGAGCGTTGACG | SEQ ID | No |
| | TTGG | NO: 2 | CTAA | NO: 19 | |
| K3 | TATGGAAAATCGAGTC | SEQ ID | GTTGGGGTGATAGGTG | SEQ ID | No |
| | ACAA | NO: 3 | CAGA | NO: 20 | |
| K4 | CTGCTTTGATGGGTAA | SEQ ID | CTATGAGTGACCCTCC | SEQ ID | No |
| | GTGT | NO: 4 | ACAC | NO: 21 | |
| K5 | TTCTTAAGATTATCTAT | SEQ ID | AGAAAGAGTAAGCCTT | SEQ ID | No |
| | GGA | NO: 5 | CACA | NO: 22 | |
| K6 | GTCACAAAGGCGAGTA | SEQ ID | TTGTATAGAATGTAAA | SEQ ID | No |
| | TGCA | NO: 6 | GAAG | NO: 23 | |
| K7 | TGCCATTCTAAAATTT | SEQ ID | AAACACAAAAAGATAT | SEQ ID | No |
| | ACCC | NO: 7 | CAGA | NO: 24 | |
| K8 | CTGTGTGGAGGGTCAC | SEQ ID | ACCTGCAGGCTCACCT | SEQ ID | No |
| | TCAT | NO: 8 | GCCA | NO: 25 | |
| K9 | TATGGAAAATCGAGTC | SEQ ID | GAAGAGGCAGTTGGGG | SEQ ID | No |
| | ACAA | NO: 9 | TGAT | NO: 26 | |
| K10 | TTTGAGATTGTGTAAC | SEQ ID | GTCCCATACGCAATCC | SEQ ID | No |
| | ACTG | NO: 10 | TAGT | NO: 27 | |
| K11 | GGTGTTTTCTTGAGGAG | SEQ ID | ACCTGCAAAAGAACTTT | SEQ ID | Yes |
| | TAG | NO: 11 | ACC | NO: 28 | |
| K12 | TCTTGAGGAGTAGAGGA | SEQ ID | ACCTGCAAAAGAACTTT | SEQ ID | Yes |
| | ACT | NO: 12 | ACC | NO: 29 | |
| K13 | CACCTGCAGGCTCACCT | SEQ ID | CTGTGTGGAGGGTCACT | SEQ ID | Yes |
| | GCC | NO: 13 | CAT | NO: 30 | |
| K14 | ACCTGCAGGCTCACCTG | SEQ ID | CTGTGTGGAGGGTCACT | SEQ ID | Yes |
| | CCA | NO: 14 | CAT | NO: 31 | |
| K15 | CTATGAGTGACCCTCCA | SEQ ID | ACTGCTGCCCACTGCTT | SEQ ID | Yes |
| | CAC | NO: 15 | TGA | NO: 32 | |
| K16 | CTATGAGTGACCCTCCA | SEQ ID | CTGCTGCCCACTGCTTT | SEQ ID | Yes |
| | CAC | NO: 16 | GAT | NO: 33 | |
| K17 | CTATGAGTGACCCTCCA | SEQ ID | CTGCTTTGATGGGTAAG | SEQ ID | Yes |
| | CAC | NO: 17 | TGT | NO: 34 | |
| K18 | GAGCTGTCAGCTTCACC | SEQ ID | GTCACTCATAGGACACC | SEQ ID | No |
| | TGC | NO: 49 | AGT | NO: 51 | |
| K19 | GAGCTGTCAGCTTCACC | SEQ ID | GGTCACTCATAGGACAC | SEQ ID | No |
| | TGC | NO: 50 | CAG | NO: 52 | |
| K20 | AAAGAGTAAGCCTTCAC | SEQ ID | GTGTTTCTTAAGATTAT | SEQ ID | No |
| | AGG | NO: 55 | CTA | NO: 56 | |

TABLE 2
Exemplary gRNAs of the disclosure targeting
exon 11 of the mouse KLKB1 gene

| First/left gRNA | | Second/right gRNA | |
|---|---|---|---|
| | SEQ ID NO: | | SEQ ID NO: |
| TACCCATTGG | SEQ ID NO: 53 | AACACAAAAA | SEQ ID NO: 54 |
| CGACCAATGA | | GATATCAGAT | |

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory, it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cpf1 gRNA, and/or a targeting domain of a gRNA.

In some embodiments the mRNA comprises a 5′ cap. As one example, the 5′ end of a gRNA can include a eukaryotic mRNA cap structure or cap analog (e.g., a G(5)ppp(5)G cap analog, a m7 G(5)ppp(5)G cap analog, or a 3′-O-Me-m7 G(5)ppp(5)G anti reverse cap analog (ARCA)), as shown below:

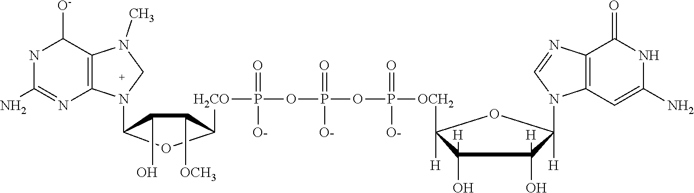

The cap or cap analog can be included during either chemical synthesis or in vitro transcription of the gRNA. In some aspects, an mRNA molecule can be capped using any method and/or capping moiety known in the art. An mRNA molecule can be capped with m7 G(5′)ppp(5′) G moiety. A m7 G(5′)ppp(5′) G moiety is also referred to herein as a “CapO”. An mRNA molecule can be capped with a CleanCap® moiety. A CleanCap® moiety can comprise a m7 G(5′)ppp(5′)(2′OMeA) (CleanCap® AG) moiety. A CleanCap® moiety can comprise a m7 G(5′)ppp(5′)(2′OMeG) (CleanCap®) GG) moiety. An mRNA molecule can be capped with an anti-reverse cap analog (ARCA®) moiety. An ARCA® moiety can comprise a m7(3′-0-methyl)G(5′)ppp(5′) G moiety. An mRNA molecule can be capped with a CleanCap® 3′OMe moiety (CleanCap®+ARCA®).

Along similar lines, the 5′ end of the gRNA can lack a 5′ triphosphate group. For instance, in vitro transcribed gRNAs can be phosphatase-treated (e.g., using calf intestinal alkaline phosphatase) to remove a 5′ triphosphate group.

Another common modification involves the addition, at the 3′ end of a gRNA, of a plurality (e.g., 1-10, 10-20, or 25-200) of adenine (A) residues referred to as a poly A tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g., a gRNA, whether transcribed in vivo from a DNA vector, or in vitro transcribed gRNA, can include either or both of a 5′ cap structure or cap analog and a 3′ poly A tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

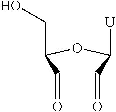

wherein “U” can be an unmodified or modified uridine.

The 3′ terminal U ribose can be modified with a 2′3′ cyclic phosphate as shown below:

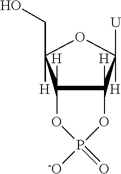

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino) propyl uridine, and 5-bromo uridine, or with any of the modified uridines described herein. In some embodiments, adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aco), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, including without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide that is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with α-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur(S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In some embodiments, the gRNA comprises one or more chemical modifications of a ribonucleotide, a ribonucleotide base, or a phosphodiester bond. In some embodiments, the one or more chemical modifications comprises at least one chemically modified phosphodiester bond. In some embodiments, the at least one chemically modified phosphodiester bond is a phosphorothioate bond.

In some embodiments, the gRNA comprises three phosphorothioate bonds at the 5-prime terminus of the gRNA. In some embodiments, the gRNA comprises two phosphorothioate bonds at the 3′ terminus of the gRNA. In some embodiments, the gRNA comprises a 2′ O-Me chemical modification at the 3′-terminus of the gRNA.

Exemplary gRNA Sequences

In some embodiments, a gRNA comprises a nucleotide sequence at least 95%, 96%, 97%, 98% or 99% (or any percentage in between) identical to SEQ ID NO: 1. In some embodiments, a gRNA comprises a nucleotide sequence of SEQ ID NO: 1.

In some embodiments, a gRNA comprises a nucleotide sequence at least 95%, 96%, 97%, 98% or 99% (or any percentage in between) identical to SEQ ID NO: 2. In some embodiments, a gRNA comprises a nucleotide sequence of SEQ ID NO: 2.

In some embodiments, a gRNA comprises a nucleotide sequence at least 95%, 96%, 97%, 98% or 99% (or any percentage in between) identical to SEQ ID NO: 3. In some embodiments, a gRNA comprises a nucleotide sequence of SEQ ID NO: 3.

In some embodiments, a gRNA comprises a nucleotide sequence at least 95%, 96%, 97%, 98% or 99% (or any percentage in between) identical to SEQ ID NO: 4. In some embodiments, a gRNA comprises a nucleotide sequence of SEQ ID NO: 4.

In some embodiments, a gRNA comprises a nucleotide sequence at least 95%, 96%, 97%, 98% or 99% (or any percentage in between) identical to SEQ ID NO: 5. In some embodiments, a gRNA comprises a nucleotide sequence of SEQ ID NO: 5.

In some embodiments, a gRNA comprises a nucleotide sequence at least 95%, 96%, 97%, 98% or 99% (or any percentage in between) identical to SEQ ID NO: 6. In some embodiments, a gRNA comprises a nucleotide sequence of SEQ ID NO: 6.

In some embodiments, a gRNA comprises a nucleotide sequence at least 95%, 96%, 97%, 98% or 99% (or any percentage in between) identical to SEQ ID NO: 7. In some embodiments, a gRNA comprises a nucleotide sequence of SEQ ID NO: 7.

In some embodiments, a gRNA comprises a nucleotide sequence at least 95%, 96%, 97%, 98% or 99% (or any percentage in between) identical to SEQ ID NO: 8. In some embodiments, a gRNA comprises a nucleotide sequence of SEQ ID NO: 8.

In some embodiments, a gRNA comprises a nucleotide sequence at least 95%, 96%, 97%, 98% or 99% (or any percentage in between) identical to SEQ ID NO: 9. In some embodiments, a gRNA comprises a nucleotide sequence of SEQ ID NO: 9.

In some embodiments, a gRNA comprises a nucleotide sequence at least 95%, 96%, 97%, 98% or 99% (or any percentage in between) identical to SEQ ID NO: 10. In some embodiments, a gRNA comprises a nucleotide sequence of SEQ ID NO: 10.

In some embodiments, a gRNA comprises a nucleotide sequence at least 95%, 96%, 97%, 98% or 99% (or any percentage in between) identical to SEQ ID NO: 11. In some embodiments, a gRNA comprises a nucleotide sequence of SEQ ID NO: 11.

In some embodiments, a gRNA comprises a nucleotide sequence at least 95%, 96%, 97%, 98% or 99% (or any percentage in between) identical to SEQ ID NO: 12. In some embodiments, a gRNA comprises a nucleotide sequence of SEQ ID NO: 12.

In some embodiments, a gRNA comprises a nucleotide sequence at least 95%, 96%, 97%, 98% or 99% (or any percentage in between) identical to SEQ ID NO: 13. In some embodiments, a gRNA comprises a nucleotide sequence of SEQ ID NO: 13.

In some embodiments, a gRNA comprises a nucleotide sequence at least 95%, 96%, 97%, 98% or 99% (or any percentage in between) identical to SEQ ID NO: 14. In some embodiments, a gRNA comprises a nucleotide sequence of SEQ ID NO: 14.

In some embodiments, a gRNA comprises a nucleotide sequence at least 95%, 96%, 97%, 98% or 99% (or any percentage in between) identical to SEQ ID NO: 15. In some embodiments, a gRNA comprises a nucleotide sequence of SEQ ID NO: 15.

In some embodiments, a gRNA comprises a nucleotide sequence at least 95%, 96%, 97%, 98% or 99% (or any percentage in between) identical to SEQ ID NO: 16. In some embodiments, a gRNA comprises a nucleotide sequence of SEQ ID NO: 16.

In some embodiments, a gRNA comprises a nucleotide sequence at least 95%, 96%, 97%, 98% or 99% (or any percentage in between) identical to SEQ ID NO: 17. In some embodiments, a gRNA comprises a nucleotide sequence of SEQ ID NO: 17.

In some embodiments, a gRNA comprises a nucleotide sequence at least 95%, 96%, 97%, 98% or 99% (or any percentage in between) identical to SEQ ID NO: 18. In some embodiments, a gRNA comprises a nucleotide sequence of SEQ ID NO: 18.

In some embodiments, a gRNA comprises a nucleotide sequence at least 95%, 96%, 97%, 98% or 99% (or any percentage in between) identical to SEQ ID NO: 19. In some embodiments, a gRNA comprises a nucleotide sequence of SEQ ID NO: 19.

In some embodiments, a gRNA comprises a nucleotide sequence at least 95%, 96%, 97%, 98% or 99% (or any percentage in between) identical to SEQ ID NO: 20. In some embodiments, a gRNA comprises a nucleotide sequence of SEQ ID NO: 20.

In some embodiments, a gRNA comprises a nucleotide sequence at least 95%, 96%, 97%, 98% or 99% (or any percentage in between) identical to SEQ ID NO: 21. In some embodiments, a gRNA comprises a nucleotide sequence of SEQ ID NO: 21.

In some embodiments, a gRNA comprises a nucleotide sequence at least 95%, 96%, 97%, 98% or 99% (or any percentage in between) identical to SEQ ID NO: 22. In some embodiments, a gRNA comprises a nucleotide sequence of SEQ ID NO: 22.

In some embodiments, a gRNA comprises a nucleotide sequence at least 95%, 96%, 97%, 98% or 99% (or any percentage in between) identical to SEQ ID NO: 23. In some embodiments, a gRNA comprises a nucleotide sequence of SEQ ID NO: 23.

In some embodiments, a gRNA comprises a nucleotide sequence at least 95%, 96%, 97%, 98% or 99% (or any percentage in between) identical to SEQ ID NO: 24. In some embodiments, a gRNA comprises a nucleotide sequence of SEQ ID NO: 24.

In some embodiments, a gRNA comprises a nucleotide sequence at least 95%, 96%, 97%, 98% or 99% (or any percentage in between) identical to SEQ ID NO: 25. In some embodiments, a gRNA comprises a nucleotide sequence of SEQ ID NO: 25.

In some embodiments, a gRNA comprises a nucleotide sequence at least 95%, 96%, 97%, 98% or 99% (or any percentage in between) identical to SEQ ID NO: 26. In some embodiments, a gRNA comprises a nucleotide sequence of SEQ ID NO: 26.

In some embodiments, a gRNA comprises a nucleotide sequence at least 95%, 96%, 97%, 98% or 99% (or any percentage in between) identical to SEQ ID NO: 27. In some embodiments, a gRNA comprises a nucleotide sequence of SEQ ID NO: 27.

In some embodiments, a gRNA comprises a nucleotide sequence at least 95%, 96%, 97%, 98% or 99% (or any percentage in between) identical to SEQ ID NO: 28. In some embodiments, a gRNA comprises a nucleotide sequence of SEQ ID NO: 28.

In some embodiments, a gRNA comprises a nucleotide sequence at least 95%, 96%, 97%, 98% or 99% (or any percentage in between) identical to SEQ ID NO: 29. In some embodiments, a gRNA comprises a nucleotide sequence of SEQ ID NO: 29.

In some embodiments, a gRNA comprises a nucleotide sequence at least 95%, 96%, 97%, 98% or 99% (or any percentage in between) identical to SEQ ID NO: 30. In some embodiments, a gRNA comprises a nucleotide sequence of SEQ ID NO: 30.

In some embodiments, a gRNA comprises a nucleotide sequence at least 95%, 96%, 97%, 98% or 99% (or any percentage in between) identical to SEQ ID NO: 31. In some embodiments, a gRNA comprises a nucleotide sequence of SEQ ID NO: 31.

In some embodiments, a gRNA comprises a nucleotide sequence at least 95%, 96%, 97%, 98% or 99% (or any percentage in between) identical to SEQ ID NO: 32. In some embodiments, a gRNA comprises a nucleotide sequence of SEQ ID NO: 32.

In some embodiments, a gRNA comprises a nucleotide sequence at least 95%, 96%, 97%, 98% or 99% (or any percentage in between) identical to SEQ ID NO: 33. In some embodiments, a gRNA comprises a nucleotide sequence of SEQ ID NO: 33.

In some embodiments, a gRNA comprises a nucleotide sequence at least 95%, 96%, 97%, 98% or 99% (or any percentage in between) identical to SEQ ID NO: 34. In some embodiments, a gRNA comprises a nucleotide sequence of SEQ ID NO: 34.

In some embodiments, a gRNA comprises a nucleotide sequence at least 95%, 96%, 97%, 98% or 99% (or any percentage in between) identical to SEQ ID NO: 49. In some embodiments, a gRNA comprises a nucleotide sequence of SEQ ID NO: 49.

In some embodiments, a gRNA comprises a nucleotide sequence at least 95%, 96%, 97%, 98% or 99% (or any percentage in between) identical to SEQ ID NO: 50. In some embodiments, a gRNA comprises a nucleotide sequence of SEQ ID NO: 50.

In some embodiments, a gRNA comprises a nucleotide sequence at least 95%, 96%, 97%, 98% or 99% (or any percentage in between) identical to SEQ ID NO: 51. In some embodiments, a gRNA comprises a nucleotide sequence of SEQ ID NO: 51.

In some embodiments, a gRNA comprises a nucleotide sequence at least 95%, 96%, 97%, 98% or 99% (or any percentage in between) identical to SEQ ID NO: 52. In some embodiments, a gRNA comprises a nucleotide sequence of SEQ ID NO: 52.

In some embodiments, a gRNA comprises a nucleotide sequence at least 95%, 96%, 97%, 98% or 99% (or any percentage in between) identical to SEQ ID NO: 53. In some embodiments, a gRNA comprises a nucleotide sequence of SEQ ID NO: 53.

In some embodiments, a gRNA comprises a nucleotide sequence at least 95%, 96%, 97%, 98% or 99% (or any percentage in between) identical to SEQ ID NO: 54. In some embodiments, a gRNA comprises a nucleotide sequence of SEQ ID NO: 54.

In some embodiments, a gRNA comprises a nucleotide sequence at least 95%, 96%, 97%, 98% or 99% (or any percentage in between) identical to SEQ ID NO: 55. In some embodiments, a gRNA comprises a nucleotide sequence of SEQ ID NO: 55.

In some embodiments, a gRNA comprises a nucleotide sequence at least 95%, 96%, 97%, 98% or 99% (or any percentage in between) identical to SEQ ID NO: 56. In some embodiments, a gRNA comprises a nucleotide sequence of SEQ ID NO: 56.

Exemplary gRNA Compositions

In certain compositions of the disclosure, a gRNA composition comprises a first gRNA and a second gRNA. In some embodiments, the first gRNA comprises a first targeting sequence. In some embodiments, the second gRNA comprises a second targeting sequence. It will be apparent to a person of skill in the art that the gRNA sequences present in Tables 1 and 2 can be freely combined to achieve optimal targeting of the construct.

In some embodiments, the composition comprises a first gRNA comprising the nucleic acid sequence of SEQ ID NO: 1 and a second gRNA comprising the nucleic acid sequence of SEQ ID NO: 18.

In some embodiments, the composition comprises a first gRNA comprising the nucleic acid sequence of SEQ ID NO: 2 and a second gRNA comprising the nucleic acid sequence of SEQ ID NO: 19.

In some embodiments, the composition comprises a first gRNA comprising the nucleic acid sequence of SEQ ID NO: 3 and a second gRNA comprising the nucleic acid sequence of SEQ ID NO: 20.

In some embodiments, the composition comprises a first gRNA comprising the nucleic acid sequence of SEQ ID NO: 4 and a second gRNA comprising the nucleic acid sequence of SEQ ID NO: 21.

In some embodiments, the composition comprises a first gRNA comprising the nucleic acid sequence of SEQ ID NO: 5 and a second gRNA comprising the nucleic acid sequence of SEQ ID NO: 22.

In some embodiments, the composition comprises a first gRNA comprising the nucleic acid sequence of SEQ ID NO: 6 and a second gRNA comprising the nucleic acid sequence of SEQ ID NO: 23.

In some embodiments, the composition comprises a first gRNA comprising the nucleic acid sequence of SEQ ID NO: 7 and a second gRNA comprising the nucleic acid sequence of SEQ ID NO: 24.

In some embodiments, the composition comprises a first gRNA comprising the nucleic acid sequence of SEQ ID NO: 8 and a second gRNA comprising the nucleic acid sequence of SEQ ID NO: 25.

In some embodiments, the composition comprises a first gRNA comprising the nucleic acid sequence of SEQ ID NO: 9 and a second gRNA comprising the nucleic acid sequence of SEQ ID NO: 26.

In some embodiments, the composition comprises a first gRNA comprising the nucleic acid sequence of SEQ ID NO: 10 and a second gRNA comprising the nucleic acid sequence of SEQ ID NO: 27.

In some embodiments, the composition comprises a first gRNA comprising the nucleic acid sequence of SEQ ID NO: 11 and a second gRNA comprising the nucleic acid sequence of SEQ ID NO: 28.

In some embodiments, the composition comprises a first gRNA comprising the nucleic acid sequence of SEQ ID NO: 12 and a second gRNA comprising the nucleic acid sequence of SEQ ID NO: 29.

In some embodiments, the composition comprises a first gRNA comprising the nucleic acid sequence of SEQ ID NO: 13 and a second gRNA comprising the nucleic acid sequence of SEQ ID NO: 30.

In some embodiments, the composition comprises a first gRNA comprising the nucleic acid sequence of SEQ ID NO: 14 and a second gRNA comprising the nucleic acid sequence of SEQ ID NO: 31.

In some embodiments, the composition comprises a first gRNA comprising the nucleic acid sequence of SEQ ID NO: 15 and a second gRNA comprising the nucleic acid sequence of SEQ ID NO: 32.

In some embodiments, the composition comprises a first gRNA comprising the nucleic acid sequence of SEQ ID NO: 16 and a second gRNA comprising the nucleic acid sequence of SEQ ID NO: 33.

In some embodiments, the composition comprises a first gRNA comprising the nucleic acid sequence of SEQ ID NO: 17 and a second gRNA comprising the nucleic acid sequence of SEQ ID NO: 34.

In some embodiments, the composition comprises a first gRNA comprising the nucleic acid sequence of SEQ ID NO: 49 and a second gRNA comprising the nucleic acid sequence of SEQ ID NO: 51.

In some embodiments, the composition comprises a first gRNA comprising the nucleic acid sequence of SEQ ID NO: 50 and a second gRNA comprising the nucleic acid sequence of SEQ ID NO: 52.

In some embodiments, the composition comprises a first gRNA comprising the nucleic acid sequence of SEQ ID NO: 53 and a second gRNA comprising the nucleic acid sequence of SEQ ID NO: 54.

In some embodiments, the composition comprises a first gRNA comprising the nucleic acid sequence of SEQ ID NO: 55 and a second gRNA comprising the nucleic acid sequence of SEQ ID NO: 56.

Exemplary Cas-CLOVER and gRNA Compositions

Gene editing compositions, including Cas-CLOVER, and methods of using these compositions for gene editing are described in detail in PCT Application Numbers PCT/US2016/037922, PCT/US2018/066941, PCT/US2017/054799, U.S. Patent Publication Nos. 2017/0107541, 2017/0114149, 2018/0187185 and U.S. Pat. No. 10,415,024, each of which is incorporated herein by reference in its entirety for examples of Cas-CLOVER constructs and other gene editing compositions that may be used in the methods described herein. In certain compositions of the disclosure, the composition comprises a first gRNA, a first fusion protein or a first polynucleotide encoding the first fusion protein (e.g. Cas-Clover), a second gRNA and a second fusion protein or a second polynucleotide encoding a second fusion protein (e.g., Cas-Clover). The first gRNA comprises a first targeting sequence. The second gRNA comprises a second targeting sequence.

In some embodiments, the first gRNA and the first fusion protein, once expressed in a cell, form a complex that localizes at the 5′ terminus of the target DNA. In some embodiments, the second gRNA and the second fusion protein form a complex that localizes at the 3′ terminus of the target DNA. In some embodiments, the first and second gRNAs are capable of targeting the fusion protein to a target region.

In some embodiments, a composition of the disclosure comprises (a) a first guide RNA (gRNA) comprising a first targeting sequence set forth in SEQ ID Nos: 1-17, 49, 50, or 55; (b) a second gRNA comprising a second targeting sequence set forth in SEQ ID NOs: 18-34, 51, 52, or 56; (c) a first polynucleotide encoding a first fusion protein, wherein the first fusion protein comprises a first inactivated Cas9 domain, or nuclease domain thereof, and a first Clo051 domain, or nuclease domain thereof; and (d) a second polynucleotide encoding a second fusion protein, wherein the second fusion protein comprises a second inactivated Cas9 domain, or nuclease domain thereof, and a second Clo051 domain, or nuclease domain thereof.

In some embodiments, the first fusion protein, the second fusion protein, or both the first and the second fusion protein comprises the amino acid sequence set forth in SEQ ID NO: 35 or SEQ ID NO: 38.

In some embodiments, the first polynucleotide encoding the first fusion protein, the second polynucleotide encoding the second fusion protein, or both the first the first polynucleotide encoding the first fusion protein and the second polynucleotide encoding the second fusion protein is an mRNA comprising the sequence set forth in SEQ ID NO: 36 or SEQ ID NO: 39.

In some embodiments, a composition of the disclosure comprises (a) a first guide RNA (gRNA) comprising a first targeting sequence set forth in SEQ ID NO: 1; (b) a second gRNA comprising a second targeting sequence set forth in SEQ ID NO: 18; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 2; (b) a second gRNA comprising a second targeting sequence set forth in SEQ ID NO: 19; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 3; (b) a second gRNA comprising a second targeting sequence set forth in SEQ ID NO: 20; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 4; (b) a second gRNA comprising a second targeting sequence set forth in SEQ ID NO: 21; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 5; (b) a second gRNA comprising a second targeting sequence set forth in SEQ ID NO: 22; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 6; (b) a second gRNA comprising a second targeting sequence set forth in SEQ ID NO: 23; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 7; (b) a second gRNA comprising a second targeting sequence set forth in SEQ ID NO: 24; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 8; (b) a second gRNA comprising a second targeting sequence set forth in SEQ ID NO: 25; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 19; (b) a second gRNA comprising a second targeting sequence set forth in SEQ ID NO: 26; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 10; (b) a second gRNA comprising a second targeting sequence set forth in SEQ ID NO: 27; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 11; (b) a second gRNA comprising a second targeting sequence set forth in SEQ ID NO: 28; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 12; (b) a second gRNA comprising a second targeting sequence set forth in SEQ ID NO: 29; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 13; (b) a second gRNA comprising a second targeting sequence set forth in SEQ ID NO: 30; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 14; (b) a second gRNA comprising a second targeting sequence set forth in SEQ ID NO: 31; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 15; (b) a second gRNA comprising a second targeting sequence set forth in SEQ ID NO: 32; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 16; (b) a second gRNA comprising a second targeting sequence set forth in SEQ ID NO: 33; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 17; (b) a second gRNA comprising a second targeting sequence set forth in SEQ ID NO: 34; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 49; (b) a second gRNA comprising a second targeting sequence set forth in SEQ ID NO: 51; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 50; (b) a second gRNA comprising a second targeting sequence set forth in SEQ ID NO: 52; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 53; (b) a second gRNA comprising a second targeting sequence set forth in SEQ ID NO: 54; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 55; (b) a second gRNA comprising a second targeting sequence set forth in SEQ ID NO: 56; (c) a first polynucleotide encoding a first fusion protein comprising the polypeptide sequence set forth in SEQ ID NO: 35, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 35, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 1; (b) a second gRNA comprising a second targeting sequence set forth in SEQ ID NO: 18; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 2; (b) a second gRNA comprising a second targeting sequence set forth in SEQ ID NO: 19; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 3; (b) a second gRNA comprising a second targeting sequence set forth in SEQ ID NO: 20; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 4; (b) a second gRNA comprising a second targeting sequence set forth in SEQ ID NO: 21; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 5; (b) a second gRNA comprising a second targeting sequence set forth in SEQ ID NO: 22; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 6; (b) a second gRNA comprising a second targeting sequence set forth in SEQ ID NO: 23; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 7; (b) a second gRNA comprising a second targeting sequence set forth in SEQ ID NO: 24; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 8; (b) a second gRNA comprising a second targeting sequence set forth in SEQ ID NO: 25; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 19; (b) a second gRNA comprising a second targeting sequence set forth in SEQ ID NO: 26; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 10; (b) a second gRNA comprising a second targeting sequence set forth in SEQ ID NO: 27; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 11; (b) a second gRNA comprising a second targeting sequence set forth in SEQ ID NO: 28; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 12; (b) a second gRNA comprising a second targeting sequence set forth in SEQ ID NO: 29; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 13; (b) a second gRNA comprising a second targeting sequence set forth in SEQ ID NO: 30; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 14; (b) a second gRNA comprising a second targeting sequence set forth in SEQ ID NO: 31; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 15; (b) a second gRNA comprising a second targeting sequence set forth in SEQ ID NO: 32; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 16; (b) a second gRNA comprising a second targeting sequence set forth in SEQ ID NO: 33; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 17; (b) a second gRNA comprising a second targeting sequence set forth in SEQ ID NO: 34; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 49; (b) a second gRNA comprising a second targeting sequence set forth in SEQ ID NO: 51; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 50; (b) a second gRNA comprising a second targeting sequence set forth in SEQ ID NO: 52; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 53; (b) a second gRNA comprising a second targeting sequence set forth in SEQ ID NO: 54; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 55; (b) a second gRNA comprising a second targeting sequence set forth in SEQ ID NO: 56; (c) a first polynucleotide encoding a first fusion protein comprising the polypeptide sequence set forth in SEQ ID NO: 38, wherein the first fusion protein comprises a first inactivated Cas9 domain, or nuclease domain thereof, and a first Clo051 domain, or nuclease domain thereof; and (d) a second polynucleotide encoding a second fusion protein comprising the polypeptide sequence set forth in SEQ ID NO: 38, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 1; (b) a second gRNA comprising a second targeting sequence set forth in SEQ ID NO: 18; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 2; (b) a second gRNA comprising a second targeting sequence set forth in SEQ ID NO: 19; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 3; (b) a second gRNA comprising a second targeting sequence set forth in SEQ ID NO: 20; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 4; (b) a second gRNA comprising a second targeting sequence set forth in SEQ ID NO: 21; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 5; (b) a second gRNA comprising a second targeting sequence set forth in SEQ ID NO: 22; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 6; (b) a second gRNA comprising a second targeting sequence set forth in SEQ ID NO: 23; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 7; (b) a second gRNA comprising a second targeting sequence set forth in SEQ ID NO: 24; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 8; (b) a second gRNA comprising a second targeting sequence set forth in SEQ ID NO: 25; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 19; (b) a second gRNA comprising a second targeting sequence set forth in SEQ ID NO: 26; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 10; (b) a second gRNA comprising a second targeting sequence set forth in SEQ ID NO: 27; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 11; (b) a second gRNA comprising a second targeting sequence set forth in SEQ ID NO: 28; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 12; (b) a second gRNA comprising a second targeting sequence set forth in SEQ ID NO: 29; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 13; (b) a second gRNA comprising a second targeting sequence set forth in SEQ ID NO: 30; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 14; (b) a second gRNA comprising a second targeting sequence set forth in SEQ ID NO: 31; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 15; (b) a second gRNA comprising a second targeting sequence set forth in SEQ ID NO: 32; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 16; (b) a second gRNA comprising a second targeting sequence set forth in SEQ ID NO: 33; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 17; (b) a second gRNA comprising a second targeting sequence set forth in SEQ ID NO: 34; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 49; (b) a second gRNA comprising a second targeting sequence set forth in SEQ ID NO: 51; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 50; (b) a second gRNA comprising a second targeting sequence set forth in SEQ ID NO: 52; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 53; (b) a second gRNA comprising a second targeting sequence set forth in SEQ ID NO: 54; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 55; (b) a second gRNA comprising a second targeting sequence set forth in SEQ ID NO: 56; (c) a first polynucleotide comprising the nucleic acid sequence set forth in SEQ ID NO: 36, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 36, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 1; (b) a second gRNA comprising a second targeting sequence set forth in SEQ ID NO: 18; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 2; (b) a second gRNA comprising a second targeting sequence set forth in SEQ ID NO: 19; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 3; (b) a second gRNA comprising a second targeting sequence set forth in SEQ ID NO: 20; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 4; (b) a second gRNA comprising a second targeting sequence set forth in SEQ ID NO: 21; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 5; (b) a second gRNA comprising a second targeting sequence set forth in SEQ ID NO: 22; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 6; (b) a second gRNA comprising a second targeting sequence set forth in SEQ ID NO: 23; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 7; (b) a second gRNA comprising a second targeting sequence set forth in SEQ ID NO: 24; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 8; (b) a second gRNA comprising a second targeting sequence set forth in SEQ ID NO: 25; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 19; (b) a second gRNA comprising a second targeting sequence set forth in SEQ ID NO: 26; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 10; (b) a second gRNA comprising a second targeting sequence set forth in SEQ ID NO: 27; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 11; (b) a second gRNA comprising a second targeting sequence set forth in SEQ ID NO: 28; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 12; (b) a second gRNA comprising a second targeting sequence set forth in SEQ ID NO: 29; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 13; (b) a second gRNA comprising a second targeting sequence set forth in SEQ ID NO: 30; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 14; (b) a second gRNA comprising a second targeting sequence set forth in SEQ ID NO: 31; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 15; (b) a second gRNA comprising a second targeting sequence set forth in SEQ ID NO: 32; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 16; (b) a second gRNA comprising a second targeting sequence set forth in SEQ ID NO: 33; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 17; (b) a second gRNA comprising a second targeting sequence set forth in SEQ ID NO: 34; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 49; (b) a second gRNA comprising a second targeting sequence set forth in SEQ ID NO: 51; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 50; (b) a second gRNA comprising a second targeting sequence set forth in SEQ ID NO: 52; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 53; (b) a second gRNA comprising a second targeting sequence set forth in SEQ ID NO: 54; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, a composition of the disclosure comprises (a) a first guide RNA (gRNA) comprising a first targeting sequence set forth in SEQ ID NO: 55; (b) a second gRNA comprising a second targeting sequence set forth in SEQ ID NO: 56; (c) a first polynucleotide comprising the nucleic acid sequence set forth in SEQ ID NO: 39, encoding a first fusion protein, wherein the first fusion protein comprises a first inactivated Cas9 domain, or nuclease domain thereof, and a first Clo051 domain, or nuclease domain thereof; and (d) a second polynucleotide comprising the nucleic acid sequence set forth in SEQ ID NO: 39, encoding a second fusion protein, wherein the second fusion protein comprises a second inactivated Cas9 domain, or nuclease domain thereof, and a second Clo051 domain, or nuclease domain thereof.

In some embodiments, the first gRNA and the first fusion protein, once expressed in a cell, form a complex that localizes at the 5′ terminus of the target DNA to be modified. In some embodiments, the second gRNA and the second fusion protein, once expressed in a cell, form a complex that localizes at the 3′ terminus of the target DNA. A schematic diagram of the composition complexed with a target DNA is shown in FIG. 10. In some embodiments, the first gRNA is encoded in an mRNA. In some embodiments, the second gRNA is encoded in an mRNA. In some embodiments, the first and second gRNA are capable of targeting the fusion protein to a target region.

In some embodiments, the C-terminus of the first inactivated Cas9 domain, or nuclease domain thereof, the second inactivated Cas9 domain, or nuclease domain thereof, or both the first inactivated Cas9 domain or nuclease domain thereof, and second inactivated Cas9 domain, or nuclease domain thereof, and the N-terminus of the first Clo051 domain, or nuclease domain thereof, the second Clo051 domain, or nuclease domain thereof, or both the first Clo051 domain or nuclease domain thereof, and second Clo051 domain, or nuclease domain thereof are connected by a linker comprising the sequence set forth in SEQ ID NO: 57.

In some embodiments, the first fusion protein, the second fusion protein, or both the first and the second fusion protein comprises an inactivated Cas9 or dCas9 derived from an S. pyogenes Cas9 polypeptide.

In some embodiments, the first gRNA, the second gRNA, or both the first and the second gRNA comprises a guide sequence and a scaffold sequence derived from an S. pyogenes Cas9 polypeptide. In some embodiments, the scaffold sequence comprise the sequence set forth in SEQ ID NO: 48. In some embodiments, the first gRNA, the second gRNA, or both the first and the second gRNA comprises one or more chemical modifications of a ribonucleotide, a ribonucleotide base, or a phosphodiester bond. In some embodiments, the chemical modification comprises a phosphorothioate bond. In some embodiments, the 5′- and/or 3′ terminus of the first gRNA, the second gRNA, or both the first and the second gRNA comprises at least two consecutive phosphorothioate bonds. In some embodiments, the 5′- and/or 3′ terminus of the first gRNA, the second gRNA, or both the first and the second gRNA comprises at least one 2′ O-Me chemical modification.

In some embodiments, the first fusion protein, the second fusion protein, or both the first and the second fusion protein are encoded in an mRNA. In some embodiments, the first and the second fusion protein are the same. In some embodiments, the first and the second fusion protein are different.

In some embodiments, the composition comprises a first gRNA comprising the nucleic acid sequence of SEQ ID NO: 1, a second gRNA comprising the nucleic acid sequence of SEQ ID NO: 18 and a fusion protein comprising the polypeptide sequence of SEQ ID NO: 35.

In some embodiments, the composition comprises a first gRNA comprising the nucleic acid sequence of SEQ ID NO: 2, a second gRNA comprising the nucleic acid sequence of SEQ ID NO: 19 and a fusion protein comprising the polypeptide sequence of SEQ ID NO: 35.

In some embodiments, the composition comprises a first gRNA comprising the nucleic acid sequence of SEQ ID NO: 3, a second gRNA comprising the nucleic acid sequence of SEQ ID NO: 20 and a fusion protein comprising the polypeptide sequence of SEQ ID NO: 35.

In some embodiments, the composition comprises a first gRNA comprising the nucleic acid sequence of SEQ ID NO: 4, a second gRNA comprising the nucleic acid sequence of SEQ ID NO: 21 and a fusion protein comprising the polypeptide sequence of SEQ ID NO: 35.

In some embodiments, the composition comprises a first gRNA comprising the nucleic acid sequence of SEQ ID NO: 5, a second gRNA comprising the nucleic acid sequence of SEQ ID NO: 22 and a fusion protein comprising the polypeptide sequence of SEQ ID NO: 35.

In some embodiments, the composition comprises a first gRNA comprising the nucleic acid sequence of SEQ ID NO: 6, a second gRNA comprising the nucleic acid sequence of SEQ ID NO: 23 and a fusion protein comprising the polypeptide sequence of SEQ ID NO: 35.

In some embodiments, the composition comprises a first gRNA comprising the nucleic acid sequence of SEQ ID NO: 7, a second gRNA comprising the nucleic acid sequence of SEQ ID NO: 24 and a fusion protein comprising the polypeptide sequence of SEQ ID NO: 35.

In some embodiments, the composition comprises a first gRNA comprising the nucleic acid sequence of SEQ ID NO: 8, a second gRNA comprising the nucleic acid sequence of SEQ ID NO: 25 and a fusion protein comprising the polypeptide sequence of SEQ ID NO: 35.

In some embodiments, the composition comprises a first gRNA comprising the nucleic acid sequence of SEQ ID NO: 9, a second gRNA comprising the nucleic acid sequence of SEQ ID NO: 26 and a fusion protein comprising the polypeptide sequence of SEQ ID NO: 35.

In some embodiments, the composition comprises a first gRNA comprising the nucleic acid sequence of SEQ ID NO: 10, a second gRNA comprising the nucleic acid sequence of SEQ ID NO: 27 and a fusion protein comprising the polypeptide sequence of SEQ ID NO: 35.

In some embodiments, the composition comprises a first gRNA comprising the nucleic acid sequence of SEQ ID NO: 11, a second gRNA comprising the nucleic acid sequence of SEQ ID NO: 28 and a fusion protein comprising the polypeptide sequence of SEQ ID NO: 35.

In some embodiments, the composition comprises a first gRNA comprising the nucleic acid sequence of SEQ ID NO: 12, a second gRNA comprising the nucleic acid sequence of SEQ ID NO: 29 and a fusion protein comprising the polypeptide sequence of SEQ ID NO: 35.

In some embodiments, the composition comprises a first gRNA comprising the nucleic acid sequence of SEQ ID NO: 13, a second gRNA comprising the nucleic acid sequence of SEQ ID NO: 30 and a fusion protein comprising the polypeptide sequence of SEQ ID NO: 35.

In some embodiments, the composition comprises a first gRNA comprising the nucleic acid sequence of SEQ ID NO: 14, a second gRNA comprising the nucleic acid sequence of SEQ ID NO: 31 and a fusion protein comprising the polypeptide sequence of SEQ ID NO: 35.

In some embodiments, the composition comprises a first gRNA comprising the nucleic acid sequence of SEQ ID NO: 15, a second gRNA comprising the nucleic acid sequence of SEQ ID NO: 32 and a fusion protein comprising the polypeptide sequence of SEQ ID NO: 35.

In some embodiments, the composition comprises a first gRNA comprising the nucleic acid sequence of SEQ ID NO: 16, a second gRNA comprising the nucleic acid sequence of SEQ ID NO: 33 and a fusion protein comprising the polypeptide sequence of SEQ ID NO: 35.

In some embodiments, the composition comprises a first gRNA comprising the nucleic acid sequence of SEQ ID NO: 17, a second gRNA comprising the nucleic acid sequence of SEQ ID NO: 34 and a fusion protein comprising the polypeptide sequence of SEQ ID NO: 35.

In some embodiments, the composition comprises a first gRNA comprising the nucleic acid sequence of SEQ ID NO: 49, a second gRNA comprising the nucleic acid sequence of SEQ ID NO: 51 and a fusion protein comprising the polypeptide sequence of SEQ ID NO: 35.

In some embodiments, the composition comprises a first gRNA comprising the nucleic acid sequence of SEQ ID NO: 50, a second gRNA comprising the nucleic acid sequence of SEQ ID NO: 52 and a fusion protein comprising the polypeptide sequence of SEQ ID NO: 35.

In some embodiments, the composition comprises a first gRNA comprising the nucleic acid sequence of SEQ ID NO: 53, a second gRNA comprising the nucleic acid sequence of SEQ ID NO: 54 and a fusion protein comprising the polypeptide sequence of SEQ ID NO: 35.

In some embodiments, the composition comprises a first gRNA comprising the nucleic acid sequence of SEQ ID NO: 55, a second gRNA comprising the nucleic acid sequence of SEQ ID NO: 56 and a fusion protein comprising the polypeptide sequence of SEQ ID NO: 35.

In some embodiments, the composition comprises a first gRNA comprising the nucleic acid sequence of SEQ ID NO: 1, a second gRNA comprising the nucleic acid sequence of SEQ ID NO: 18 and a fusion protein comprising the polypeptide sequence of SEQ ID NO: 38.

In some embodiments, the composition comprises a first gRNA comprising the nucleic acid sequence of SEQ ID NO: 2, a second gRNA comprising the nucleic acid sequence of SEQ ID NO: 19 and a fusion protein comprising the polypeptide sequence of SEQ ID NO: 38.

In some embodiments, the composition comprises a first gRNA comprising the nucleic acid sequence of SEQ ID NO: 3, a second gRNA comprising the nucleic acid sequence of SEQ ID NO: 20 and a fusion protein comprising the polypeptide sequence of SEQ ID NO: 38.

In some embodiments, the composition comprises a first gRNA comprising the nucleic acid sequence of SEQ ID NO: 4, a second gRNA comprising the nucleic acid sequence of SEQ ID NO: 21 and a fusion protein comprising the polypeptide sequence of SEQ ID NO: 38.

In some embodiments, the composition comprises a first gRNA comprising the nucleic acid sequence of SEQ ID NO: 5, a second gRNA comprising the nucleic acid sequence of SEQ ID NO: 22 and a fusion protein comprising the polypeptide sequence of SEQ ID NO: 38.

In some embodiments, the composition comprises a first gRNA comprising the nucleic acid sequence of SEQ ID NO: 6, a first fusion protein comprising the polypeptide sequence of SEQ ID NO: 38 a second gRNA comprising the nucleic acid sequence of SEQ ID NO: 23 and a fusion protein comprising the polypeptide sequence of SEQ ID NO: 38.

In some embodiments, the composition comprises a first gRNA comprising the nucleic acid sequence of SEQ ID NO: 7, a second gRNA comprising the nucleic acid sequence of SEQ ID NO: 24 and a fusion protein comprising the polypeptide sequence of SEQ ID NO: 38.

In some embodiments, the composition comprises a first gRNA comprising the nucleic acid sequence of SEQ ID NO: 8, a second gRNA comprising the nucleic acid sequence of SEQ ID NO: 25 and a fusion protein comprising the polypeptide sequence of SEQ ID NO: 38.

In some embodiments, the composition comprises a first gRNA comprising the nucleic acid sequence of SEQ ID NO: 9, a second gRNA comprising the nucleic acid sequence of SEQ ID NO: 26 and a fusion protein comprising the polypeptide sequence of SEQ ID NO: 38.

In some embodiments, the composition comprises a first gRNA comprising the nucleic acid sequence of SEQ ID NO: 10, a second gRNA comprising the nucleic acid sequence of SEQ ID NO: 27 and a fusion protein comprising the polypeptide sequence of SEQ ID NO: 38.

In some embodiments, the composition comprises a first gRNA comprising the nucleic acid sequence of SEQ ID NO: 11, a second gRNA comprising the nucleic acid sequence of SEQ ID NO: 28 and a fusion protein comprising the polypeptide sequence of SEQ ID NO: 38.

In some embodiments, the composition comprises a first gRNA comprising the nucleic acid sequence of SEQ ID NO: 12, a second gRNA comprising the nucleic acid sequence of SEQ ID NO: 29 and a fusion protein comprising the polypeptide sequence of SEQ ID NO: 38.

In some embodiments, the composition comprises a first gRNA comprising the nucleic acid sequence of SEQ ID NO: 13, a second gRNA comprising the nucleic acid sequence of SEQ ID NO: 30 and a fusion protein comprising the polypeptide sequence of SEQ ID NO: 38.

In some embodiments, the composition comprises a first gRNA comprising the nucleic acid sequence of SEQ ID NO: 14, a second gRNA comprising the nucleic acid sequence of SEQ ID NO: 31 and a fusion protein comprising the polypeptide sequence of SEQ ID NO: 38.

In some embodiments, the composition comprises a first gRNA comprising the nucleic acid sequence of SEQ ID NO: 15, a second gRNA comprising the nucleic acid sequence of SEQ ID NO: 32 and a fusion protein comprising the polypeptide sequence of SEQ ID NO: 38.

In some embodiments, the composition comprises a first gRNA comprising the nucleic acid sequence of SEQ ID NO: 16, a second gRNA comprising the nucleic acid sequence of SEQ ID NO: 33 and a fusion protein comprising the polypeptide sequence of SEQ ID NO: 38.

In some embodiments, the composition comprises a first gRNA comprising the nucleic acid sequence of SEQ ID NO: 17, a second gRNA comprising the nucleic acid sequence of SEQ ID NO: 34 and a fusion protein comprising the polypeptide sequence of SEQ ID NO: 38.

In some embodiments, the composition comprises a first gRNA comprising the nucleic acid sequence of SEQ ID NO: 49, a second gRNA comprising the nucleic acid sequence of SEQ ID NO: 51 and a fusion protein comprising the polypeptide sequence of SEQ ID NO: 38.

In some embodiments, the composition comprises a first gRNA comprising the nucleic acid sequence of SEQ ID NO: 50, a second gRNA comprising the nucleic acid sequence of SEQ ID NO: 52 and a fusion protein comprising the polypeptide sequence of SEQ ID NO: 38.

In some embodiments, the composition comprises a first gRNA comprising the nucleic acid sequence of SEQ ID NO: 53, a second gRNA comprising the nucleic acid sequence of SEQ ID NO: 54 and a fusion protein comprising the polypeptide sequence of SEQ ID NO: 38.

In some embodiments, the composition comprises a first gRNA comprising the nucleic acid sequence of SEQ ID NO: 55, a second gRNA comprising the nucleic acid sequence of SEQ ID NO: 56 and a fusion protein comprising the polypeptide sequence of SEQ ID NO: 38.

Delivery of gRNAs and Genetic Editing Compositions

The compositions described herein can be delivered to a cell using any suitable method known in the art or described herein. For example, the gRNAs and polynucleotides encoding a fusion protein can be present on a transposon or a vector. The compositions, transposons, and vectors can be delivered using, for example, lipid nanoparticles.

### Lipid Nanoparticles

The compositions of the present disclosure may be encapsulated in at least one lipid nanoparticle comprising at least one cationic lipid. In some aspects, a cationic lipid can be a bioreducible ionizable cationic lipid. In some aspects, the at least one or more compositions of the present disclosure can be formulated in a lipid nanoparticle. In some aspects, a lipid nanoparticle can further comprise at least one structural lipid. In some aspects, a lipid nanoparticle can further comprise at least one phospholipid. In some aspects, a lipid nanoparticle can further comprise at least one PEGylated lipid.

Accordingly, the present disclosure provides compositions comprising at least one lipid nanoparticle, wherein the at least one lipid nanoparticle comprises at least one cationic lipid, at least one composition of the present disclosure, at least one structural lipid, at least one phospholipid and at least one PEGylated lipid. In some aspects, the lipid nanoparticle can comprise lipid and one or more nucleic acids of the present disclosure at a specified ratio (weight/weight).

The compositions disclosed herein can also be delivered to cells or target tissues using one or more lipid nanoparticle compositions and methods of making the same, as described in PCT Application No. PCT/US2023/061005 and PCT Publication No. WO 2022/182792, which is incorporated herein by reference in its entirety for examples of lipid nanoparticles that may be used to deliver the compositions disclosed herein to their target.

### Bioreducible Ionizable Cationic Lipids

In some aspects, a cationic lipid can be a bioreducible ionizable cationic lipid. Accordingly, the compositions of the present disclosure can be encapsulated at least one lipid nanoparticle, wherein the at least one lipid nanoparticle comprises at least one bioreducible ionizable cationic lipid.

As used herein, the term “bioreducible ionizable cationic lipid” is used in its broadest sense to refer to a cationic lipid comprising: at least one tertiary amine, at least one disulfide group, at least one group comprising a bond that is susceptible to cleavage by thioesterification, and further comprising at least two saturated or unsaturated hydrocarbon chains. Exemplary bioreducible ionizable cationic lipids include, but are not limited to, those described in Akita et al., (2020) Biol. Phar. Bull. 43:1617-1625, the contents of which are incorporated herein by reference in their entirety.

Additional exemplary bioreducible ionizable cationic lipids and methods of preparing such lipids useful in the methods of the present disclosure include those disclosed in International Patent Application No. PCT/JP2016/052690, published as WO/2016/121942 and International Patent Application No. PCT/JP2019/012302, published as WO/2019/188867, the contents of each of which are incorporated herein by reference in their entirety for examples of lipid nanoparticles that may be used to deliver the compositions disclosed herein to their target.

Accordingly, compositions of the present disclosure may be encapsulated in at least one lipid nanoparticle, wherein the at least one lipid nanoparticle comprises any one of the bioreducible ionizable cationic lipids put forth in WO/2016/121942 and WO/2019/188867.

The bioreducible ionizable cationic lipids of the present disclosure are biodegradable, thereby allowing the bioreducible ionizable cationic lipids to be broken down and metabolized in an animal. Without wishing to be bound by theory, this bioreducibility advantageously lessens cationic lipid-associated cytotoxicity.

In some aspects of the compositions and methods of the present disclosure, a bioreducible ionizable cationic lipid for use in the LNP compositions can be ssPalmO-Ph-P4C2. As would be appreciated by the skilled artisan, ssPalmO-Ph-P4C2 has the following structure:

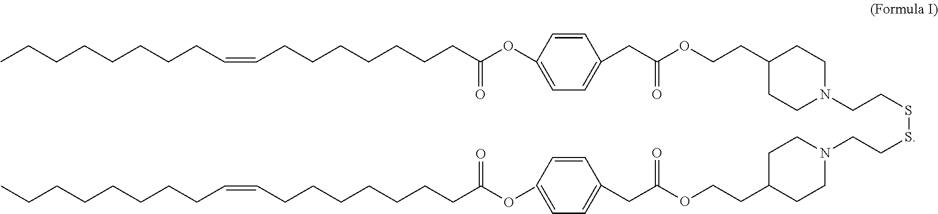

As would be appreciated by the skilled artisan, ssPalmO-Ph-P4C2 can also be referred to as Coatsome® SS-OP, ssPalmO-Phe-P4C2, ssPalmO-Phenyl-P4C2, ssPalmO-Phe and ssPalmO-Ph. Accordingly, ssPalmO-Ph-P4C2, Coatsome® SS-OP, ssPalmO-Phe-P4C2, ssPalmO-Phenyl-P4C2, ssPalmO-Phe and ssPalmO-Ph are used interchangeably herein to refer to the bioreducible ionizable cationic lipid with the chemical structure put forth in Formula I. Gene editing tools can also be delivered to cells using one or more poly(histidine)-based micelles. Poly(histidine) (e.g., poly(L-histidine)), is a pH-sensitive polymer due to the imidazole ring providing an electron lone pair on the unsaturated nitrogen. That is, poly(histidine) has amphoteric properties through protonation-deprotonation. In particular, at certain pHs, poly(histidine)-containing triblock copolymers may assemble into a micelle with positively charged poly(histidine) units on the surface, thereby enabling complexing with the negatively charged gene editing molecule(s). Using these nanoparticles to bind and release proteins and/or nucleic acids in a pH-dependent manner may provide an efficient and selective mechanism to perform a desired gene modification. In particular, this micelle-based delivery system provides substantial flexibility with respect to the charged materials, as well as a large payload capacity, and targeted release of the nanoparticle payload. In one example, site-specific cleavage of the double stranded DNA is enabled by delivery of a composition disclosed herein using the poly(histidine)-based micelles. Without wishing to be bound by a particular theory, it is believed that in the micelles that are formed by the various triblock copolymers, the hydrophobic blocks aggregate to form a core, leaving the hydrophilic blocks and poly(histidine) blocks on the ends to form one or more surrounding layer.

Without wishing to be bound by theory, three specific segments of ssPalmO-Ph-P4C2 are hypothesized to facilitate its biodegradation. First, the tertiary amine of each piperidine ring is an acidic pH-responsive cation-charging unit. Upon endocytosis, the tertiary amine moieties become positively charged in response to the acidic, intracellular endosomal compartment. These are now able to interact and destabilize the membrane and this leads to endosomal escape. Once in the cytosol, the disulfide bond is susceptible to reduction by glutathione generating two free sulfhydryl groups. The resulting high concentration of free thiols further leads to nucleophilic reaction and the particle undergoes self-degradation/collapse via thioesterification and releases the payload in the cytosol. This is defined as HyPER or Hydrolysis accelerated by the intra-Particle Enrichment of Reactant and potentially eliminates the potentially toxic side effects of cationic lipids in general.

In an aspect, the disclosure provides triblock copolymers made of a hydrophilic block, a hydrophobic block, and a charged block. In some aspects, the hydrophilic block may be poly(ethylene oxide) (PEO), and the charged block may be poly(L-histidine). An example tri-block copolymer that can be used is a PEO-b-PLA-b-PHIS, with variable numbers of repeating units in each block varying by design.

Diblock copolymers that can be used as intermediates for making triblock copolymers can have hydrophilic biocompatible poly(ethylene oxide) (PEO), which is chemically synonymous with PEG, coupled to various hydrophobic aliphatic poly(anhydrides), poly(nucleic acids), poly(esters), poly(ortho esters), poly(peptides), poly(phosphazenes) and poly(saccharides), including but not limited by poly(lactide) (PLA), poly(glycolide) (PLGA), poly(lactic-co-glycolic acid) (PLGA), poly(ε-caprolactone) (PCL), and poly(trimethylene carbonate) (PTMC). Polymeric micelles comprised of 100% PEGylated surfaces possess improved in vitro chemical stability, augmented in vivo bioavailability, and prolonged blood circulatory half-lives.

Polymeric vesicles, polymersomes and poly(Histidine)-based micelles, including those that comprise triblock copolymers, and methods of making the same, are described in further detail in U.S. Pat. Nos. 7,217,427; 7,868,512; 6,835,394; 8,808,748; 10,456,452; U.S. Publication Nos. 2014/0363496; 2017/0000743; and 2019/0255191; and PCT Publication No. WO 2019/126589, each of which is incorporated herein by reference in its entirety for examples of lipid nanoparticles that may be used to deliver the compositions disclosed herein to their target.

In some aspects, a composition of the present disclosure is encapsulated in at least one lipid nanoparticle comprising: about 40.75% of a terpene lipidoid compound by moles, about 51.75% of cholesterol by moles, about 5% of DOPC by moles, and about 2.5% of DMG-PEG2000 by moles, wherein a polynucleotide encoding the mutant Cas-Clover is a RNA molecule, and wherein the ratio of lipid to RNA molecule in the at least one nanoparticle is about 120:1 (w/w).

In some aspects, the terpene lipidoid compound is HMA-404:

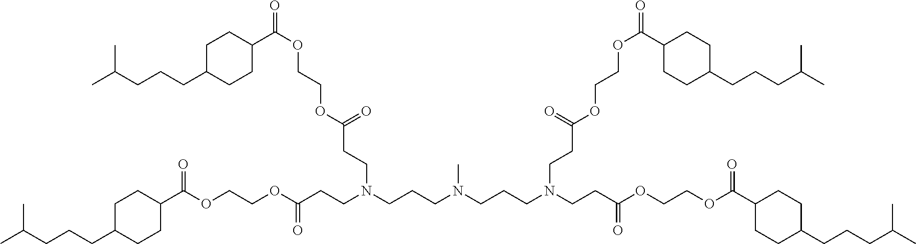 [SMILES: CC(C)CCCC1CCC(C(=O)OCCOC(=O)CCN(CCCN(C)CCCN(CCC(=O)OCCOC(=O)C2CCC(CCCC(C)C)CC2)CCC(=O)OCCOC(=O)C2CCC(CCCC(C)C)CC2)CCC(=O)OCCOC(=O)C2CCC(CCCC(C)C)CC2)CC1]

Accordingly, in some aspects, a composition of the present disclosure is encapsulated in at least one lipid nanoparticle comprising: about 40.75% of HMA-404 by moles, about 51.75% of cholesterol by moles, about 5% of DOPC by moles, and about 2.5% of DMG-PEG2000 by moles, wherein a polynucleotide encoding the mutant Cas-Clover is a RNA molecule, and wherein the ratio of lipid to RNA molecule in the at least one nanoparticle is about 120:1 (w/w).

In some aspects, a composition of the present disclosure is encapsulated in at least one lipid nanoparticle comprising: about 54% of SS-OP by moles, about 35% of cholesterol by moles, about 5% of DOPC by moles, about 5% of DSPC by moles, and about 1% of DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

In some aspects, a composition of the present disclosure is encapsulated in at least one lipid nanoparticle comprising at least one nucleic acid molecule can comprise about 54% SS-OP by moles, about 35% cholesterol by moles, about 10% DOPE by moles, and about 1% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 100:1 (w/w).

### General Procedure for Synthesis of Lipidoids (E)

### General Formula E.1

The general structure of E.1 compounds is shown below.

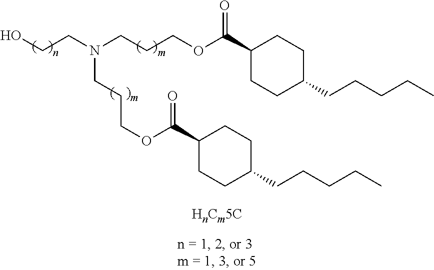

The synthetic route of E. 1 compounds is given in the following General Scheme E.1. This two-step sequence begins with an esterification reaction between trans-4-pentylcyclohexane carboxylic acid and hydroxy substituted alkyl bromides of different lengths (C3, C5, and C7) catalyzed by N-Ethyl-N′-(3-dimethylaminopropyl) carbodiimide hydrochloride (EDC-HCl) and N,N-Dimethylpyridin-4-amine (DMAP). The corresponding ester which bears a bromide as a functional handle reacts with hydroxy substituted amines (Hₙ, where n=2, 3, or 4) to give the target compounds.

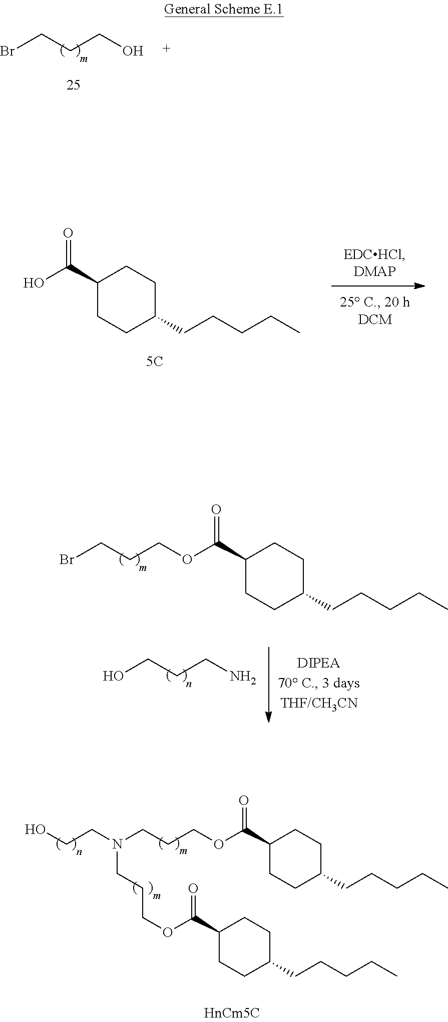

### Preparation of Compound No. 37

COMPOUND NO. 37 was prepared in accordance with the General Scheme (E.1).

Following the general protocol for amine alkylation described in General Scheme E.1, amine H3 (22 mg) was combined with BC6B5C (442 mg) and DIPEA (200 μL) in THF/CH3CN (1:1, 0.8 mL). After the reaction, the crude was purified by 6% MeOH/DCM eluants. Brown oil, 127 mg (36%); 1H NMR (499 MHz, CDCl₃) δ 4.12 (t, J=4.8 Hz, 12H), 3.81-3.75 (m, 2H), 2.65 (s, 2H), 2.39 (td, J=12.8, 6.8 Hz, 5H), 2.32 (t, J=7.5 Hz, 4H), 2.22 (tt, J=12.2, 3.6 Hz, 4H), 1.98-1.91 (m, 8H), 1.84-1.76 (m, 8H), 1.72-1.60 (m, 7H), 1.50 (s, 4H), 1.44-1.13 (m, 49H), 0.95-0.84 (m, 20H); LC-MS: Rt 9.504 min, m/z calculated [M+H]: 1200.92, found 1200.75.

In some aspects, COMPOUND NO. 37 comprises the following structure:

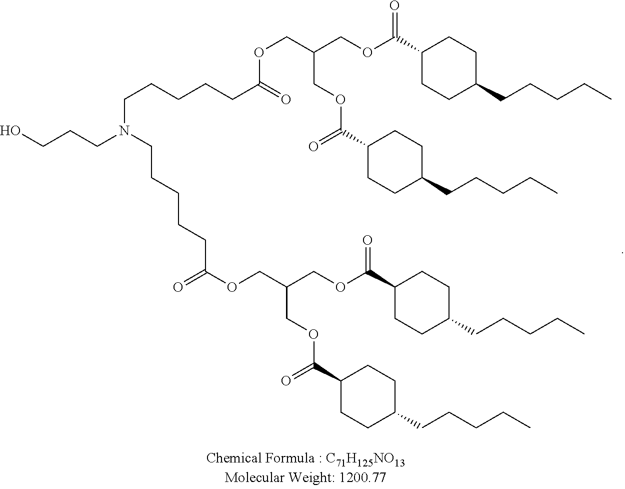 [SMILES: CCCCC[C@H]1CC[C@H](C(=O)OCC(COC(=O)CCCCCN(CCCO)CCCCCC(=O)OCC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1]

In some aspects, a composition of the present disclosure is encapsulated in at least one lipid nanoparticle comprising at least one nucleic acid molecule can comprise about 50% COMPOUND NO. 37 by moles, about 10% DSPC by moles, about 38.5% cholesterol by moles, and about 1.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w).

In some aspects, a composition of the present disclosure is encapsulated in at least one lipid nanoparticle comprising at least one nucleic acid molecule can comprise about 50% COMPOUND NO. 37 by moles, about 10% DOPC by moles, about 38% cholesterol by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w).

In some aspects, a composition of the present disclosure is encapsulated in at least one lipid nanoparticle comprising at least one nucleic acid molecule can comprise about 50% COMPOUND NO. 37 by moles, about 10% DSPC by moles, about 38.5% cholesterol by moles, and about 1.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w). In some aspects, the ratio of lipid to nucleic acid in the at least one nanoparticle can be about 40:1 (w/w).

In some aspects, a composition of the present disclosure is encapsulated in at least one lipid nanoparticle comprising at least one nucleic acid molecule can comprise about 50% COMPOUND NO. 37 by moles, about 10% DPPC by moles, about 38.5% cholesterol by moles, and about 1.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w). In some aspects, the ratio of lipid to nucleic acid in the at least one nanoparticle can be about 40:1 (w/w).

In some aspects, a composition of the present disclosure is encapsulated in at least one lipid nanoparticle comprising at least one nucleic acid molecule can comprise about 50% COMPOUND NO. 37 by moles, about 10% DSPC by moles, about 38% cholesterol by moles, and about 2% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w). In some aspects, the ratio of lipid to nucleic acid in the at least one nanoparticle can be about 40:1 (w/w).

In some aspects, a composition of the present disclosure is encapsulated in at least one lipid nanoparticle comprising at least one nucleic acid molecule can comprise about 50% COMPOUND NO. 37 by moles, about 10% DPPC by moles, about 38% cholesterol by moles, and about 2% DMG-PEG2000 by moles, wherein the lipid nanoparticle further comprises at least one mRNA molecule. In some aspects, a composition of the present disclosure is encapsulated in at least one lipid nanoparticle comprising at least one nucleic acid molecule can comprise about 50% COMPOUND NO. 37 by moles, about 10% DSPC by moles, about 38% cholesterol by moles, and about 2% DMG-PEG2000 by moles, wherein the lipid nanoparticle further comprises at least one mRNA molecule. In some aspects, the composition is encapsulated in at least one lipid nanoparticle comprising at least one nucleic acid molecule can comprise about 50% COMPOUND NO. 37 by moles, about 5% DSPC by moles, about 42% cholesterol by moles, and about 3% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w). In some aspects, the ratio of lipid to nucleic acid in the at least one nanoparticle can be about 40:1 (w/w).

In some aspects the lipid compound is COMPOUND NO. 1:

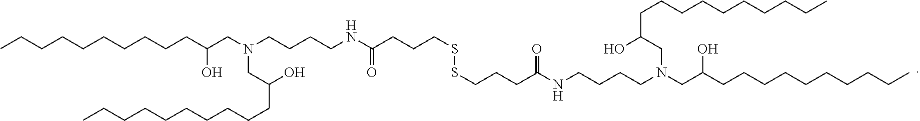 [SMILES: CCCCCCCCCCC(O)CN(CCCCNC(=O)CCCSSCCCC(=O)NCCCCN(CC(O)CCCCCCCCCC)CC(O)CCCCCCCCCC)CC(O)CCCCCCCCCC]

In some aspects, a composition of the present disclosure is encapsulated in at least one lipid nanoparticle comprising at least one nucleic acid molecule can comprise about 50% COMPOUND NO. 1 by moles, about 10% DOPC by moles, about 38.5% cholesterol by moles, and about 1.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 50:1 (w/w). In some aspects, the ratio of lipid to nucleic acid in the at least one nanoparticle can be about 60:1 (w/w). In some aspects, the ratio of lipid to nucleic acid in the at least one nanoparticle can be about 80:1 (w/w).

In some aspects, a composition of the present disclosure is encapsulated in at least one lipid nanoparticle comprising at least one nucleic acid molecule can comprise about 40% COMPOUND NO. 1 by moles, about 10% DOPC by moles, about 48.5% cholesterol by moles, and about 1.5% DMG-PEG2000 by moles, wherein the lipid nanoparticle further comprises at least one mRNA molecule. In some aspects, the mRNA molecule further comprises a 5′-CAP. In some aspects, the ratio of lipid to nucleic acid in the at least one nanoparticle can be about 60:1 (w/w).

As described herein, the LNPs encapsulating a composition of the present disclosure that comprises at least one bioreducible ionizable cationic lipid advantageously exhibit significantly reduced toxicity in animals as compared to LNPs comprising non-bioreducible ionizable cationic lipids. In particular, administration of the LNPs of the present disclosure surprisingly does not result in any body weight loss. In some aspects, certain LNP compositions of the present disclosure are sufficiently non-toxic that animals administered the LNPs actually gain body weight, even when administered amounts of LNPs that exceed the lethal dose of LNP comprising non-bioreducible ionizable cationic lipids.

### LNP Components

The LNPs disclosed herein may comprise one or more structural lipids, one or more phospholipids, and/or one or more pegylated lipids.

### Structural Lipids

In some aspects, a structural lipid can be a steroid. In some aspects, a structural lipid can be a sterol. In some aspects, a structural lipid can comprise cholesterol. In some aspects, a structural lipid can comprise ergosterol. In some aspects, a structural lipid can be a phytosterol.

### Phospholipid

As used herein, the term “phospholipid” is used in its broadest sense to refer to any amphiphilic molecule that comprises a polar (hydrophilic) headgroup comprising phosphate and two hydrophobic fatty acid chains.

In some aspects of the LNPs of the present disclosure, a phospholipid can comprise dioleoylphosphatidylethanolamine (DOPE).

In some aspects of the LNPs of the present disclosure, a phospholipid can comprise DOPC (1,2-Dioleoyl-sn-glycero-3-phosphocholine).

In some aspects of the LNPs of the present disclosure, a phospholipid can comprise DSPC (1,2-Distearoyl-sn-glycero-3-phosphocholine).

### PEGylated Lipid

As used herein, the term “PEGylated lipid” is used to refer to any lipid that is modified (e.g., covalently linked to) at least one polyethylene glycol molecule. In some aspects, a PEGylated lipid can comprise 1,2-dimyristoyl-rac-glycero-3-methoxypoly ethylene glycol-2000, hereafter referred to as DMG-PEG2000.

### Cells and Modified Cells of the Disclosure

In another aspect, provided herein are cells that are modified using the compositions and methods disclosed herein. In preferred embodiments, the cells are modified to reduce the expression of KLKB1 protein.

Cells and modified cells of the disclosure can be mammalian cells. The cells and modified cells are human cells. In some embodiments, the cells can comprise hematopoietic progenitor cells (HPCs). In some embodiments, the cells can comprise Hematopoietic stem cells (HSCs). In some embodiments, the cells are hematopoietic stem and precursor cells (HSPCs). In some embodiments, the HSPCs are capable of differentiating into erythroid progenitor cells. In certain embodiments, at least a portion of the plurality of cells may be within an erythroid lineage. In some embodiments, the HSPC is capable of differentiating into an erythroid progenitor cell.

Cells that have been altered ex vivo according to this disclosure can be manipulated (e.g., expanded, passaged, frozen, differentiated, de-differentiated, transduced with a transgene, etc.) prior to their delivery to a subject. The cells can be delivered to a subject from which they are obtained (in an “autologous” transplant), or to a recipient who is immunologically distinct from a donor of the cells (in an “allogeneic” transplant). In some embodiments, the cell is not a germ cell. In some embodiments, the cell is not a human germ cell.

In some embodiments, an autologous transplant includes the steps of obtaining, from the subject, a plurality of cells, either circulating in peripheral blood, or within the marrow or other tissue (e.g., spleen, skin, etc.), and manipulating those cells to enrich for cells in the erythroid lineage (e.g., by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting the KLKB1 gene. The genome editing system can be implemented or delivered to the cells in any suitable format, including as a ribonucleoprotein complex, as separated protein and nucleic acid components, and/or as nucleic acids encoding the components of the genome editing system.

The cells, following delivery of the genome editing system, are optionally manipulated e.g., to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

### Modified Cells of the Disclosure

The disclosure provides a method of modifying a population of cells comprising contacting the population of cells with the compositions of the disclosure (e.g., first Cas-Clover fusion protein and first gRNA, and second Cas-Clover fusion protein and second gRNA compositions), wherein the first gRNA forms a complex with the first targeting sequence and the first fusion protein, and the second gRNA forms a complex with the second targeting sequence and the second fusion protein, thereby generating an indel between the first targeting sequence and the second targeting sequence and producing a modified population of cells. In some aspects, the targeting sequence is at the KLKB1 gene. In some aspects, the targeting sequence is within Exons 6 to 12 of the KLKB1 gene. In some embodiments, the indel is generated at the KLKB1 gene. In some embodiments, the indel causes inactivation of the KLKB1 gene.

The disclosure provides methods of using a disclosed composition or pharmaceutical composition for modifying the genomes of a population of cells. In some embodiments the method of modifying the genomes of a population of cells comprises contacting the population of cells with one or more compositions of the present disclosure wherein the first and second fusion proteins are expressed by each cell of the population, wherein the first gRNA is bound to the first fusion protein and the second gRNA is bound to the second fusion protein, wherein the first gRNA specifically binds to a first strand of a first double-stranded DNA target sequence in each cell of the population, and wherein the second gRNA specifically binds to a second strand of a second double-stranded DNA target sequence in each cell of the population.

In some embodiments, the first fusion protein and the second fusion protein introduces a modification into the genome of one or more cells in the population. In some embodiments, the modification is an insertion or deletion (indel) between the first double-stranded DNA target sequence and the second double-stranded DNA target sequence. In some embodiments, the indel causes the inactivation of a KLKB1 gene.

The disclosure also provides a population of cells modified according to the method of the present disclosure. In some embodiments, the population of cells has a reduced level of Klkb1 protein expression relative to an unmodified population of cells

In some embodiments, the disclosure relates to compositions including a plurality of cells generated by the method disclosed herein, in which at least 20%, 30%, 40%, 50%, 60%, 70%, 80% or 90% of the cells comprise an indel between the first targeting sequence and the second targeting sequence. In some embodiments, the disclosure relates to compositions including a plurality of cells generated by the method disclosed herein, in which 20-90%, 30-80%, 40-70%, or 50-60% of cells include an indel between the first targeting sequence and the second targeting sequence. In some embodiments, the disclosure relates to compositions including a plurality of cells generated by the method disclosed herein, in which about 20%, 30%, 40%, 50%, 60%, 70%, 80% or 90% of the cells comprise an indel between the first targeting sequence and the second targeting sequence. In some embodiments, the first targeting sequence, the second targeting sequence, or the first and second targeting sequence is at the KLKB1 gene.

In some embodiments, the disclosure relates to compositions including a plurality of cells generated by the method disclosed herein, in which at least 20%, 30%, 40%, 50%, 60%, 70%, 80% or 90% of the cells comprise an indel at the targeting sequences. In some embodiments, the disclosure relates to compositions including a plurality of cells generated by the method disclosed herein, in which 20-90%, 30-80%, 40-70%, or 50-60% of the cells comprise an indel at the targeting sequences. In some embodiments, the disclosure relates to compositions including a plurality of cells generated by the method disclosed herein, in which about 20%, 30%, 40%, 50%, 60%, 70%, 80% or 90% of the cells comprise an indel at the targeting sequences. In some embodiments, the targeting sequences are at the KLKB1 gene.

In some embodiments, the disclosure relates to compositions including a plurality of cells generated by the method disclosed herein, in which at least 20%, 30%, 40%, 50%, 60%, 70%, 80% or 90% of the cells comprise an edited KLKB1 gene. In some embodiments, the disclosure relates to compositions including a plurality of cells generated by the method disclosed herein, in which 20-90%, 30-80%, 40-70%, or 50-60% of cells comprise an edited KLKB1 gene. In some embodiments, the disclosure relates to compositions including a plurality of cells generated by the method disclosed herein, in which about 20%, 30%, 40%, 50%, 60%, 70%, 80% or 90% of the cells comprise an edited KLKB1 gene.

In some embodiments, the disclosure relates to compositions including a plurality of cells generated by the method disclosed herein, in which there is at least a 20%, 30%, 40%, 50%, 60%, 70%, 80%, 90%, 95%, or 99% reduction of KLKB1 protein relative to an unmodified population of cells. In some embodiments, the disclosure relates to compositions including a plurality of cells generated by the method disclosed herein, in which there is a 20-99%, 30-95%, 40-90%, or 60-80% reduction of KLKB1 protein relative to an unmodified population of cells. In some embodiments, the disclosure relates to compositions including a plurality of cells generated by the method disclosed herein, in which about 20%, 30%, 40%, 50%, 60%, 70%, 80% 90%, 99%, or 100% reduction of KLKB1 protein relative to an unmodified population of cells.

The unmodified population of cells may be, for example, a population of cells prior to modification by one or gene editing compositions of the present disclosure, or a population of cells that did not receive a gene composition of the present disclosure.

Cells and modified immune cells of the disclosure can be autologous cells or allogeneic cells. Allogeneic cells are engineered to prevent adverse reactions to engraftment following administration to a subject. Allogeneic cells may be any type of cell. Allogenic cells can be stem cells or can be derived from stem cells. Allogeneic cells can be differentiated somatic cells.

### Formulations, Dosages and Modes of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material that releases the incorporated components by diffusion. The components can be homogeneously or heterogeneously distributed within the release system. A variety of release systems can be useful, however, the choice of the appropriate system will depend upon the rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Methods of Using the Compositions of the Disclosure

The disclosure provides the use of a disclosed composition or pharmaceutical composition for the treatment of a disease or disorder in a cell, tissue, organ, animal, or subject, as known in the art or as described herein, using the disclosed compositions and pharmaceutical compositions, e.g., administering or contacting the cell, tissue, organ, animal, or subject with a therapeutic effective amount of the composition or pharmaceutical composition. In one aspect, the subject is a mammal. Preferably, the subject is human. The terms “subject” and “patient” are used interchangeably herein.

The disclosure provides a method for modulating or treating at least one disease or disorder in a cell, tissue, organ, animal or subject. Preferably, the malignant disease is a Hereditary Angioedema. Non-limiting examples of a Hereditary Angioedema include Type I and Type II.

The disclosure provides the use of a composition of the present disclosure for the manufacture of a medicament for the treatment of Hereditary Angioedema.

The compositions of the disclosure may be used to treat a disease or disorder by use of a therapeutic transgene encoding for an exogenous nucleic acid sequence or exogenous amino acid sequence. For certain diseases or disorders, the therapeutic transgene can include KLKB1.

### Definitions

As used throughout the disclosure, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more standard deviations. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The disclosure provides isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various aspects, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the disclosure or biologically active portion thereof is recombinantly produced, optimally culture medium represents less than about 30%, 20%, 10%, 5%, or 1% (by dry weight) of chemical precursors or non-protein-of-interest chemicals.

Nucleic acids or proteins of the disclosure can be constructed by a modular approach including preassembling monomer units and/or repeat units in target vectors that can subsequently be assembled into a final destination vector. Polypeptides of the disclosure may comprise repeat monomers of the disclosure and can be constructed by a modular approach by preassembling repeat units in target vectors that can subsequently be assembled into a final destination vector. The disclosure provides polypeptides produced by this method as well as nucleic acid sequences encoding these polypeptides. The disclosure provides host organisms and cells comprising nucleic acid sequences encoding polypeptides produced by this modular approach.

The term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of shall mean excluding more than trace elements of other ingredients and substantial method steps. Aspects defined by each of these transition terms are within the scope of this disclosure.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. “Gene expression” refers to the conversion of the information, contained in a gene, into a gene product. A gene product can be the direct transcriptional product of a gene (e.g., mRNA, tRNA, rRNA, antisense RNA, ribozyme, shRNA, micro RNA, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” or “regulation” of gene expression refers to a change in the activity of a gene. Modulation of expression can include, but is not limited to, gene activation and gene repression.

The term “operatively linked” or its equivalents (e.g., “linked operatively”) means two or more molecules are positioned with respect to each other such that they are capable of interacting to affect a function attributable to one or both molecules or a combination thereof.

A “target site” or “target sequence” or “targeting sequence” is a nucleic acid sequence that defines a portion of a nucleic acid to which a binding molecule will bind, provided sufficient conditions for binding exist.

Nucleic acids of the disclosure may be single- or double-stranded. Nucleic acids of the disclosure may contain double-stranded sequences even when the majority of the molecule is single-stranded. Nucleic acids of the disclosure may contain single-stranded sequences even when the majority of the molecule is double-stranded. Nucleic acids of the disclosure may include genomic DNA, cDNA, RNA, or a hybrid thereof. Nucleic acids of the disclosure may contain combinations of deoxyribo- and ribo-nucleotides. Nucleic acids of the disclosure may contain combinations of bases including uracil, adenine, thymine, cytosine, guanine, inosine, xanthine hypoxanthine, isocytosine and isoguanine. Nucleic acids of the disclosure may be synthesized to comprise non-natural amino acid modifications. Nucleic acids of the disclosure may be obtained by chemical synthesis methods or by recombinant methods.

Nucleic acids of the disclosure, either their entire sequence, or any portion thereof, may be non-naturally occurring. Nucleic acids of the disclosure may contain one or more mutations, substitutions, deletions, or insertions that do not naturally occur, rendering the entire nucleic acid sequence non-naturally occurring. Nucleic acids of the disclosure may contain one or more duplicated, inverted or repeated sequences, the resultant sequence of which does not naturally occur, rendering the entire nucleic acid sequence non-naturally occurring, Nucleic acids of the disclosure may contain modified, artificial, or synthetic nucleotides that do not naturally occur, rendering the entire nucleic acid sequence non-naturally occurring.

Given the redundancy in the genetic code, a plurality of nucleotide sequences may encode any particular protein. All such nucleotide sequences are contemplated herein.

A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. Amino acids of similar hydropathic indexes can be substituted and still retain protein function. In an aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits the calculation of the greatest local average hydrophilicity of that peptide, a useful measure that has been reported to correlate well with antigenicity and immunogenicity. U.S. Pat. No. 4,554,101, incorporated fully herein by reference.

Substitution of amino acids having similar hydrophilicity values can result in peptides retaining biological activity, for example, immunogenicity. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

As used herein, “conservative” amino acid substitutions may be defined as set out in Tables A, B, or C below. In some aspects, fusion polypeptides and/or nucleic acids encoding such fusion polypeptides include conservative substitutions that have been introduced by modification of polynucleotides encoding polypeptides of the disclosure. Amino acids can be classified according to physical properties and contribution to secondary and tertiary protein structure. A conservative substitution is a substitution of one amino acid for another amino acid that has similar properties. Exemplary conservative substitutions are set out in Table 33.

TABLE 33
Conservative Substitutions I

| Side chain characteristics | | Amino Acid |
|---|---|---|
| Aliphatic | Non-polar | G A P I L V F |
| | Polar-uncharged | C S T M N Q |
| | Polar-charged | D E K R |

| | |
|---|---|
| Aromatic | H F W Y |
| Other | N Q D E |

Alternately, conservative amino acids can be grouped as described in Lehninger, (Biochemistry, Second Edition; Worth Publishers, Inc. NY, N.Y. (1975), pp. 71-77) as set forth in Table 34.

TABLE 34
Conservative Substitutions II

| Side Chain Characteristic | | Amino Acid |
|---|---|---|
| Non-polar | Aliphatic: | A L I V P |
| (hydrophobic) | Aromatic: | F W Y |
| | Sulfur-containing: | M |
| | Borderline: | G Y |
| Uncharged-polar | Hydroxyl: | S T Y |
| | Amides: | N Q |
| | Sulfhydryl: | C |
| | Borderline: | G Y |

| | |
|---|---|
| Positively Charged (Basic): | K R H |
| Negatively Charged (Acidic): | D E |

Alternately, exemplary conservative substitutions are set out in Table 35.

Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the polypeptides of the disclosure are intended to include polypeptides bearing one or more insertions, deletions, or substitutions, or any combination thereof, of amino acid residues as well as modifications other than insertions, deletions, or substitutions of amino acid residues. Polypeptides or nucleic acids of the disclosure may contain one or more conservative substitutions.

Polypeptides and proteins of the disclosure, either their entire sequence, or any portion thereof, may be non-naturally occurring. Polypeptides and proteins of the disclosure may contain one or more mutations, substitutions, deletions, or insertions that do not naturally occur, rendering the entire amino acid sequence non-naturally occurring. Polypeptides and proteins of the disclosure may contain one or more duplicated, inverted or repeated sequences, the resultant sequence of which does not naturally occur, rendering the entire amino acid sequence non-naturally occurring. Polypeptides and proteins of the disclosure may contain modified, artificial, or synthetic amino acids that do not naturally occur, rendering the entire amino acid sequence non-naturally occurring.

As used throughout the disclosure, “sequence identity” may be determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety). The terms “identical” or “identity” when used in the context of two or more nucleic acids or polypeptide sequences, refer to a specified percentage of residues that are the same over a specified region of each of the sequences. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of a single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used throughout the disclosure, the term “endogenous” refers to nucleic acid or protein sequences naturally associated with a target gene or a host cell into which it is introduced.

As used throughout the disclosure, the term “exogenous” refers to a nucleic acid or protein sequence not naturally associated with a target gene or a host cell into which it is introduced, including non-naturally occurring multiple copies of a naturally occurring nucleic acid, e.g., DNA sequence, or naturally occurring nucleic acid sequence located in a non-naturally occurring genome location.

The disclosure provides methods of introducing a polynucleotide construct comprising a DNA sequence into a host cell. By “introducing” is intended to present to the cell the polynucleotide construct in such a manner that the construct gains access to the interior of the host cell. The methods of the disclosure do not depend on a particular method for introducing a polynucleotide construct into a host cell, only that the polynucleotide construct gains access to the interior of one cell of the host. Methods for introducing polynucleotide constructs into bacteria, plants, fungi and animals are known in the art including, but not limited to, stable transformation methods, transient transformation methods, and virus-mediated methods.

As used herein, the term “subject” refers to any animal, preferably a human patient, livestock, or other domesticated animal.

As used herein, the term “isolated” or the like refers to a cell, or a population of cells, which has been separated from its original environment, i.e., the environment of the isolated cells is substantially free of at least one component as found in the environment in which the “un-isolated” reference cells exist. The term includes a cell that is removed from some or all components as it is found in its natural environment, for example, tissue, or biopsy. The term also includes a cell that is removed from at least one, some or all components as the cell is found in non-naturally occurring environments, for example, culture, or cell suspension. Therefore, an isolated cell is partly or completely separated from at least one component, including other substances, cells or cell populations, as it is found in nature or as it is grown, stored or subsisted in non-naturally occurring environments. Specific examples of isolated cells include partially pure cells, substantially pure cells and cells cultured in a medium that is non-naturally occurring. Isolated cells may be obtained from separating the desired cells, or populations thereof, from other substances or cells in the environment, or from removing one or more other cell populations or subpopulations from the environment. As used herein, the term “purify” or the like refers to increased purity. For example, the purity can be increased to at least 50%, 60%, 70%, 80%, 90%, 95%, 99%, or 100%.

A “therapeutically effective amount”, as used herein, includes within its meaning a non-toxic but sufficient and/or effective amount of the particular therapeutic and/or pharmaceutical composition to which it is referring to provide a desired therapeutic effect. The exact amount required will vary from subject to subject depending on factors such as the patient's general health, the patient's age and the stage and severity of the condition. In particular embodiments, a therapeutically sufficient amount is sufficient and/or effective to ameliorate, reduce, and/or improve at least one symptom associated with a disease or condition of the subject being treated.

### EXAMPLES

### Example 1: Design and Synthesis of gRNA Pairs Targeting Exons 6-12 of the Human KLKB1 Gene

A set of 20 gRNA pairs targeting Exons 6-12 of the human KLKB1 gene were designed and the targeting sequence of each gRNA is shown in Table 1.

The 20 gRNA pairs comprising the targeting sequences of Table 1 were synthesized, purified, and resuspended in RNAse free water and stored at −70° C. until use.

### Example 2: Preparation of 5′-Capped mRNA Encoding Cas-CLOVER for Encapsulation in LNP Compositions

The following is a non-limiting example demonstrating the preparation of an exemplary mRNA encoding Cas-CLOVER that may be incorporated in LNP compositions for use in combination with methods including AAV donor DNA polynucleotides and vectors of the present disclosure.

The DNA plasmid “pRTb_Cas-CLOVERv3” encodes Cas-CLOVERv3 (amino acid sequence-SEQ ID NO: 38). The DNA plasmid “pRTb_Cas-CLOVERv3” encodes a Cas-CLOVERv3 comprising an N-terminal SV40 nuclear localization signal (NLS) and containing the 5′ UTR of the human beta-globin gene (HBB) and the 3′ UTR of the human cytochrome b-245 alpha chain gene (CYBA). This plasmid was used as a template for in vitro transcription reactions to produce mRNA encoding Cas-CLOVERv3 further comprising a 5′-CAP. This plasmid was used as a template for in vitro transcription reactions to produce mRNA encoding Cas-CLOVERv3 further comprising a 5′-CAP (full length mRNA sequence-SEQ ID NO: 39).

Briefly, approximately 300 μg of supercoiled pRTb_Cas-CLOVERv3 was added to a 15 mL conical comprising 300 μL CutSmart® Buffer, 60 μL of the restriction enzyme Bbsl-HF in 3000 μL total volume. The plasmid DNA was linearized by incubating at 37° C. overnight to ensure complete digestion.

The linearized plasmid was purified using a DNA QIAquick PCR purification kit according to the manufacturer's instructions, and the purified DNA was eluted in 900 μL of nuclease-free water. The DNA concentration and purity of the eluate were determined using a NanoDrop® microvolume spectrophotometer in accordance with the manufacturer's instructions.

The purified plasmid was used as a DNA template to produce mRNA using the custom in vitro transcription mMESSAGE mMACHINE T7 Transcription Kit in accordance with internal, Quality-controlled manufacturing batch records. Briefly, 100 mM stocks of the nucleotides GTP, ATP, CTP, and N1MeΨTP (N1-Methylpseudouridine-5′-Triphosphate) and CleanCap Reagent AG (m7 G(5′)ppp(5′)(2′OMeA)pG; Trilink®) were prepared. 1,485 μL each of ATP, UTP, and 5MeC and 1,188 μL each of GTP and CleanCap® Reagent AG were blended.

For the IVT reaction, 153 μg of linearized pRTb_Cas-CLOVERv3 DNA, 1,800 μL of 10×T7 RXN Buffer, 1,800 μL of T7 Enzyme mix, and 6,831 μL of the NTP and cap blend were added to a 50 mL conical (18,000 μL final volume) and incubated at 37° C. for 3 hours. A 900 μL aliquot of DNaseI enzyme was added and the tube was further incubated at 37° C. for 15 min to degrade the DNA template.

A poly(A) tail was post-enzymatically added to the 3′ end of the S′-CleanCap®-Cas-CLOVER-N1MeΨ mRNA. 18 mL of 5×EPAP Buffer, 9 mL of 25 mM MnCl₂, 9 mL ATP Solution, and 3,000 μL E-PAP, were added to the IVT reaction (90,000 μL total volume), and incubated at 37° C. for 1 hour. The bulk E-PAP reaction was subsequently divided into three 125 mL PETG bottles in 30 mL aliquots.

The 5′-CleanCap®-Cas-CLOVER-poly(A)-N1MeΨ mRNA was purified using an RNeasy Maxi Purification Kit according to the manufacturer's instructions. Briefly, a working stock of Buffer RLT was formulated using 178.2 mL of Buffer RLT with 1.8 mL of 2-mercaptoethanol. 52.2 mL of the BME+RLT solution and 37.8 mL of 100% EtOH were added to each 30 mL mRNA aliquot. The purified mRNA product was eluted in 52.5 mL of nuclease-free water, and the bulk product was stored at −80° C. The DNA linearization, IVT, and mRNA purification process is repeated until the target yield is reached.

Bulk mRNA lots were analyzed using gel electrophoresis before combination in a 500 mL PETG bottle and sampled for concentration readings using the NanoDrop®. Lithium Chloride 5× was added to the pooled mRNA in the amount of ⅓ of the total volume of the mRNA, then divided into equal 40 mL aliquots in 50 mL conical tubes and incubated at −20° C. for 45 minutes. Directly following incubation, the conical tubes are centrifuged at 14,000×g for 30 minutes at 4° C. The mRNA pellet is washed using 70% EtOH three times.

The washed mRNA pellets are dried, then resuspended in nuclease-free water. The mRNA concentration was determined using the NanoDrop®, and additional nuclease-free water was added as necessary to further dilute the product to the target concentration. The mRNA is sterile filtered using a 0.22 μm PES SteriCup® Filter before the final mRNA concentration and purity are measured on the NanoDrop®.

### Example 3: In Vitro Cas-CLOVER Editing of Exons 6-12 of Human KLKB1 Gene in Huh7 Cell Line

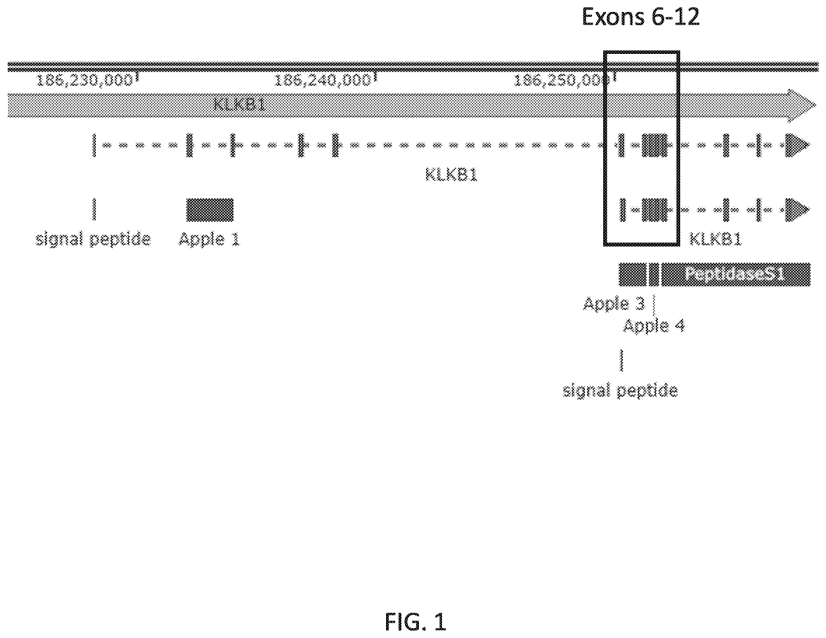

gRNA pairs targeting Exons 6-12 of the human KLKB1 gene (Table 1) were screened in Huh7 cell line to determine the percent of Cas-CLOVER editing of the KLKB1 locus for each gRNA pair (FIG. 1). Briefly, 50,000 Huh7 cells were transfected with 5 μg of an mRNA encoding Cas-CLOVER v2 (full length mRNA sequence (SEQ ID NO: 36) encodes Cas-CLOVER v2 (SEQ ID NO: 35)) and 4 μg of each gRNA pair using lipofectamine, and cultured in DMEM media supplemented with 10% FBS. After 48 hours, the medium was removed, the cells were lysed and genomic DNA was isolated from the cell extracts using a QuickExtract™ Kit in accordance with the manufacturer's instructions. The percentage of indels at the KLKB1 locus was determined by amplicon-seq for each of the gRNA pairs.

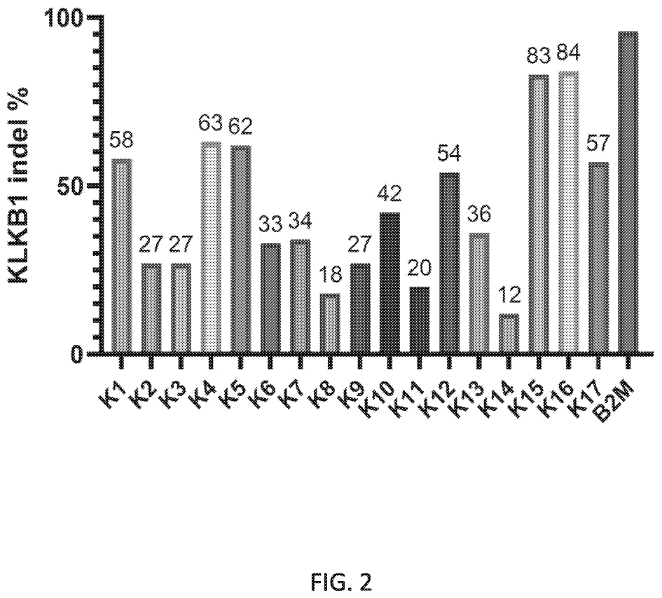

Briefly, genomic DNA samples were subjected to PCR amplification using DNA primers flanking the edit site at the KLKB1 gene that further contain Illumina partial adapters. The resulting PCR amplicons underwent a second PCR reaction using primers containing Illumina P5 and P7 sequences (Illumina Corp) and a unique index sequence (New England Biolabs). The final amplicons were pooled at equimolar concentrations and analyzed using a Miseq benchtop sequencer following standard procedures for Amplicon-seq according to the manufacturer (Illumina Corp). Sequence data were analyzed using a CRISPResso2 program to determine the frequency of indels in each sample. The results are shown in Table 3 and FIG. 2.

TABLE 3
Percent Indels at KLKB1 locus using Cas-
CLOVERv2 and targeted gRNA pairs

| gRNA Pair # | % Indels |
|---|---|
| 1 | 58 |
| 2 | 27 |
| 3 | 27 |
| 4 | 63 |
| 5 | 62 |
| 6 | 33 |
| 7 | 34 |
| 8 | 18 |
| 9 | 27 |
| 10 | 42 |
| 11 | 20 |
| 12 | 54 |
| 13 | 36 |
| 14 | 12 |
| 15 | 83 |
| 16 | 84 |
| 17 | 57 |
| 18 | 45 |
| 19 | 64 |
| 20 | 43 |

As shown in Table 3 and FIG. 2, each of the gRNA pairs was capable of targeting Cas-CLOVER to the KLKB1 locus with a range of activities from 12% to 84%, with several of the gRNA pairs demonstrating greater than 50% gene editing of the KLKB1 locus.

### Example 4: In Vitro Cas-CLOVER Editing of Exons 6-12 of Human KLKB1 Gene in HepaRG Cell Line

A subset of the gRNA pairs targeting Exons 6-12 of the human KLKB1 gene, gRNA Pairs #1, 4, 5, 15, and 16, were screened in HepaRG cell line to determine the percent of Cas-CLOVER editing of the KLKB1 locus for each of the five gRNA pairs. Briefly, 450,000 HepaRG cells were transfected with 5 μg of an mRNA encoding Cas-CLOVER v2 (full length mRNA sequence (SEQ ID NO: 36) encodes Cas-CLOVER v2 (SEQ ID NO: 35)) and 4 μg of each gRNA pair using lipofectamine, and cultured in DMEM medium supplemented with 10% FBS. After 48 hours, the medium was removed, the cells were lysed, and genomic DNA was isolated from the cell extracts using a QuickExtract™ Kit in accordance with the manufacturer's instructions. The percentage of indels at the KLKB1 locus was determined by amplicon-seq for each of the gRNA pairs.

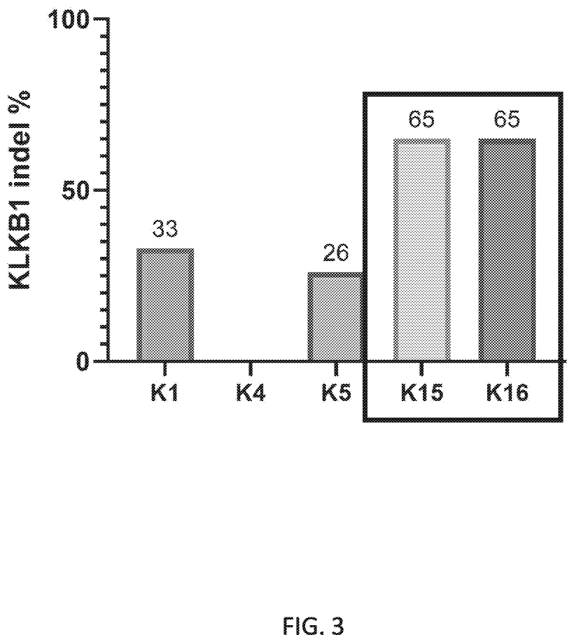

Briefly, genomic DNA samples were subjected to PCR amplification using DNA primers flanking the edit site at the KLKB1 gene that further contain Illumina partial adapters. The resulting PCR amplicons underwent a second PCR reaction using primers containing Illumina P5 and P7 sequences (Illumina Corp) and a unique index sequence (New England Biolabs). The final amplicons were pooled at equimolar concentrations and analyzed using a Miseq benchtop sequencer following standard procedures for Amplicon-seq according to the manufacturer (Illumina Corp). Sequence data were analyzed using a CRISPResso2 program to determine the frequency of indels in each sample. The results are shown in Table 4 and FIG. 3.

TABLE 4
Percent Indels at KLKB1 locus using Cas-
CLOVERv2 and targeted gRNA pairs

| gRNA Pair # | % Indels |
|---|---|
| 1 | 33 |
| 5 | 26 |
| 15 | 65 |
| 16 | 65 |

As shown in Table 4, gRNA Pairs 15 and 16 produced the highest number of percent indels of the human KLKB1 locus in HepaRG cell line.

### Example 5: Preparation of an LNP Composition Comprising a 5′-Capped mRNA Encoding Cas-CLOVER and a gRNA Pair

The following is a non-limiting example that provides exemplary methods for formulating an LNP composition comprising a 5′-capped mRNA encoding Cas-CLOVER and a KLKB1 gRNA pair for use in combination to edit the KLKB1 gene.

Individual 25 mg/ml stock solutions were prepared by solubilizing the lipids in 200-proof HPLC-grade ethanol and stock solutions were stored at −80° C. until formulated. At the time of formulation, the lipid stock solutions were briefly allowed to equilibrate to room temperature and then placed on a hot plate maintained at a temperature range of 50-55° C. Subsequently, the hot lipid stock solutions were combined to yield the desired final molar percentages.

A 1 mg/ml solution of the 5′CleanCap-N1-CC mRNA prepared in Example 2 to be incorporated into the LNPs was added to 150 mM sodium acetate buffer (pH 5.2) to form a stock solution and kept on ice. A 1 mg/ml solution of the gRNA Pair dissolved in RNAse free water to be incorporated into the LNPs was added to 150 mM sodium acetate buffer (pH 5.2) to form a gRNA stock solution and kept on ice. mRNA and gRNA stock solutions were mixed at a 3:1 ratio to form a nucleic acid stock solution. The lipid phase was mixed with the aqueous mRNA/gRNA phase inside a microfluidic chip using a NanoAssemblr® instrument according to the manufacturer's instructions to form LNP compositions comprising encapsulated Cas-CLOVER mRNAs and a targeting gRNA pair. Nanoassemblr® process parameters for mRNA encapsulation were at a flow rate of 20 ml/min and at a lipid:RNA ratio (v/v) of 1:3.

The resultant Cas-CLOVER mRNA-gRNA pair LNP compositions were then transferred to a Repligen Float-A-Lyzer dialysis device-having a molecular weight cut off (MWCO) of 8-10 kDa and processed by dialysis against 25 mM sodium acetate (dialysate:dialysis buffer volume at least 1:200 v/v), pH 5.5 overnight at 4° C. (or alternatively room temperature for at least 4 hours), to remove the 25% ethanol and achieve a complete buffer exchange. Where applicable, the LNP compositions were further concentrated using an Amicon® Ultra-4 centrifugal filter unit, MWCO-30 kDa spun at ~4100×g in an ultracentrifuge. Sucrose was added to a final concentration of 5% (w/v) to the mRNA LNPs which were then stored at 4° C. or frozen at −80° C. until further use. The average particle size diameter of the LNPs ranged from approximately 85-105 nm.

### Example 6: Editing of Human KLKB1 Locus in Cultured Primary Human Hepatocytes Using LNP Compositions Comprising a KLKB1 Targeting gRNA Pair and an mRNA Encoding Cas-CLOVER

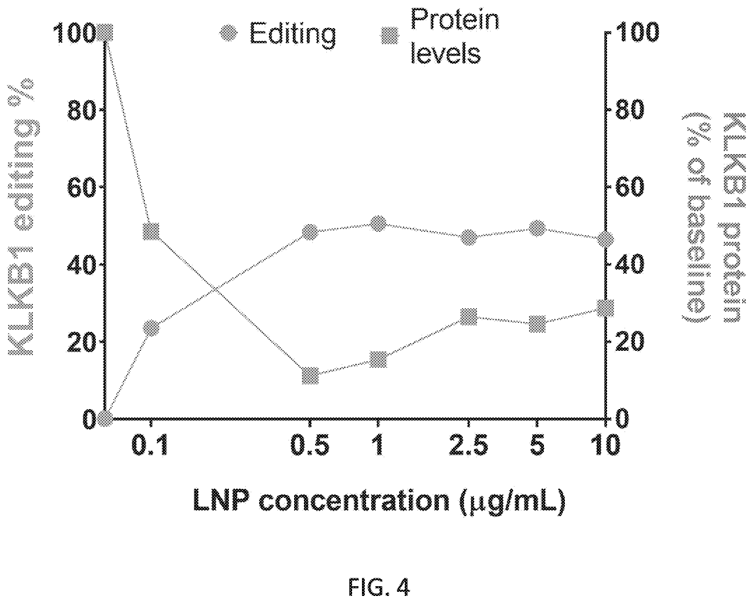

In a first experiment, approximately 450,000 cultured primary human hepatocytes were incubated with 0.005, 0.01, 0.5, 0.1, 0.5, or 1.0 μg/ml of the LNP composition of Example 5 comprising KLKB1 targeting gRNA Pair 16 (SEQ ID NO: 16 and SEQ ID NO: 33) and an mRNA encoding Cas-CLOVERv3 (SEQ ID NO: 39) in DMEM medium supplemented with 10% FBS. After 72 hours, the medium was removed, the cells were lysed and genomic DNA was isolated from the cell extracts using a QuickExtract Kit (Lucigen Corp.) in accordance with the manufacturer's instructions. The percentage indels at the KLKB1 locus was determined by amplicon-seq for each of the 6 LNP concentrations as described in Example 3. In addition, KLKB1 protein levels were calculated for each LNP concentration using a KLKB1 ELISA assay to determine the percent decrease in KLKB1 protein levels compared to levels in unedited baseline cells. The results are shown in FIG. 4.

As shown in FIG. 4, a dose response was observed for percent KLKB1 editing as well as KLKB1 protein levels in cells edited using increasing concentrations of LNP composition, with greater than 45% edited KLKB1 loci and a greater than 75% reduction in KLKB1 protein levels using 0.5 g/ml of LNP composition.

In a second experiment, approximately 450,000 cultured primary human hepatocytes were incubated with 1.0 μg/ml of the LNP composition of Example 5 comprising KLKB1 targeting gRNA Pair #K16, Pair #K18, Pair #K19 and Pair #K₂O and an mRNA encoding Cas-CLOVERv3.0 (SEQ ID NO: 39) in DMEM medium supplemented with 10% FBS. After 72 hours, the medium was removed, the cells were lysed, and genomic DNA was isolated from the cell extracts using a QuickExtract™ Kit in accordance with the manufacturer's instructions. The percentage indels at the KLKB1 locus was determined by amplicon-seq for each of the gRNA pairs as described in Example 3. In addition, KLKB1 protein levels were calculated for each LNP concentration using a KLKB1 ELISA assay to determine the percent decrease in KLKB1 protein levels compared to levels in unedited baseline cells. The results are shown in Table 5.

TABLE 5
Percent Protein Expression and Percent Indels at KLAB1
locus using Cas-CLOVERv3 and targeted gRNA pairs

| gRNA Pair # | % Indels | % KLKB1 Protein |
|---|---|---|
| K16 | 47 | 32 |
| K18 | 49 | 38 |
| K19 | 50 | 41 |
| K20 | 36 | 30 |

As shown in Table 5, all four gRNA pairs targeting the KLKB1 resulted in appreciable editing of the KLKB1 gene with 36%-50% editing per haploid genome and a resulting decrease in the percent of KLKB1 protein expression between 59% and 70% compared to unedited KLKB1 protein levels.

### Example 7: Editing of Human KLKB1 Locus in Liver Humanized Mice

LNP compositions comprising 54% Coatsome-SS-OP by moles; 35% Cholesterol by moles, 10% DOPE by moles and 1% DMG-PEG by moles, the gRNA Pair #16 targeting human KLKB1 locus, and an mRNA encoding Cas-CLOVER v2 (SEQ ID NO: 36) or an mRNA encoding Cas-CLOVER v3.0 (SEQ ID NO: 39) were prepared for delivering editing composition to the liver of mice. The two versions of the Cas-CLOVER mRNA comprise the different coding sequences and also differ in the non-coding sequence components and base modifications. For instance, Cas-CLOVER v2 comprises a wild type Cas-CLOVER sequence and 5′methyl-cytosine (5MeC) replacing cytosines whereas Cas-CLOVERv3 comprises a 5′-UTR, a S44P mutation in the Cas-CLOVER coding sequence, 3′ 2×CYBA elements and N1-pseudouridine base modifications.

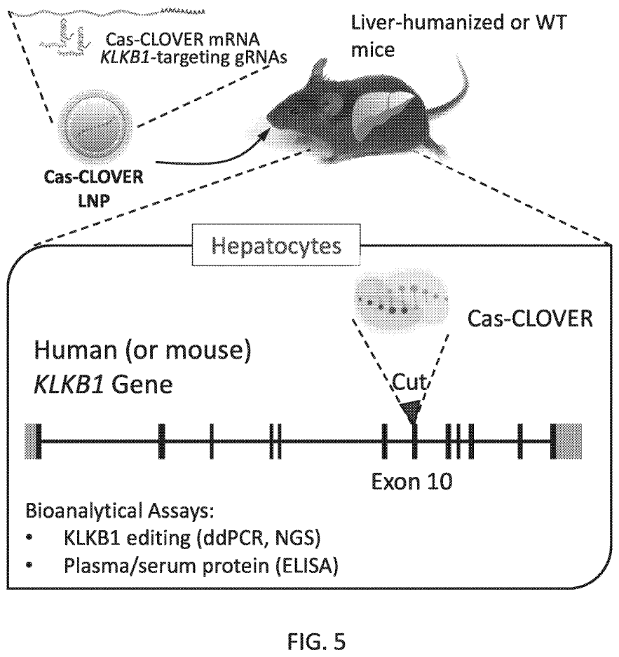

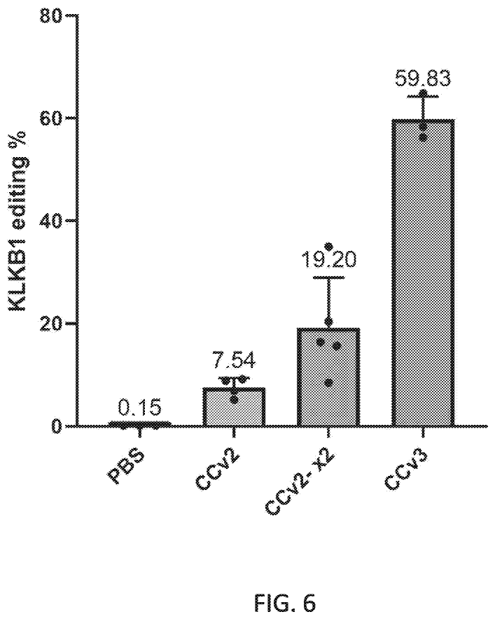

TK-Nog liver humanized mice were divided into four groups (n=3/group) and were intravenously administered: vehicle (control), 2 mg/kg of LNP Cas-CLOVER v2, two administrations of 2 mg/kg of LNP Cas-CLOVER v2, or 2 mg/kg of LNP Cas-CLOVERv3 (FIG. 5). After seven days, mice were euthanized, and liver tissue was resected and flash frozen in liquid nitrogen. To isolate genomic DNA, liver samples were mixed with lysis buffer (15 mg of tissue in 200 μL of lysis buffer+10 μL Proteinase K) and pulverized in a TissueLyser II using Triple-Pure zirconium beads. Homogenized tissue was then incubated at 56° C. for 30 minutes and column-purified using a Monarch Genomic DNA Purification kit in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a NanoDrop® device. DNA samples were used for KLKB1 editing quantification determined by amplicon-seq for control and treated mice as described in Example 3. The results are shown in Table 6 and FIG. 6.

TABLE 6

| Dose | % Indels |
|---|---|
| 0 | 0.15 |
| 2 mg/kg Cas-CLOVER v2 | 7.54 |
| 2 × 2 mg/kg Cas-CLOVER v2 | 19.20 |
| 2 mg/kg Cas-CLOVER v3 | 59.83 |

As shown in Table 6, Cas-CLOVER v2 editing was lower than Cas-CLOVER v3, but re-dosing Cas-CLOVER v2 resulted in increased editing levels compared to a single dose administration.

### Example 8: Editing of Murine Klkb1 Locus in Male and Female Adult Wild Type Mice

LNP compositions comprising 54% Coatsome-SS-OP by moles; 35% Cholesterol by moles, 10% DOPE by moles and 1% DMG-PEG by moles, a gRNA pair targeting murine Klkb1 locus identified in a murine gRNA screen (SEQ ID NO: 53 and SEQ ID NO: 54), and an mRNA encoding Cas-CLOVER v2 (SEQ ID NO: 36) or an mRNA encoding Cas-CLOVER v3 (SEQ ID NO: 39) were prepared as described herein for delivering editing composition to the liver of mice.

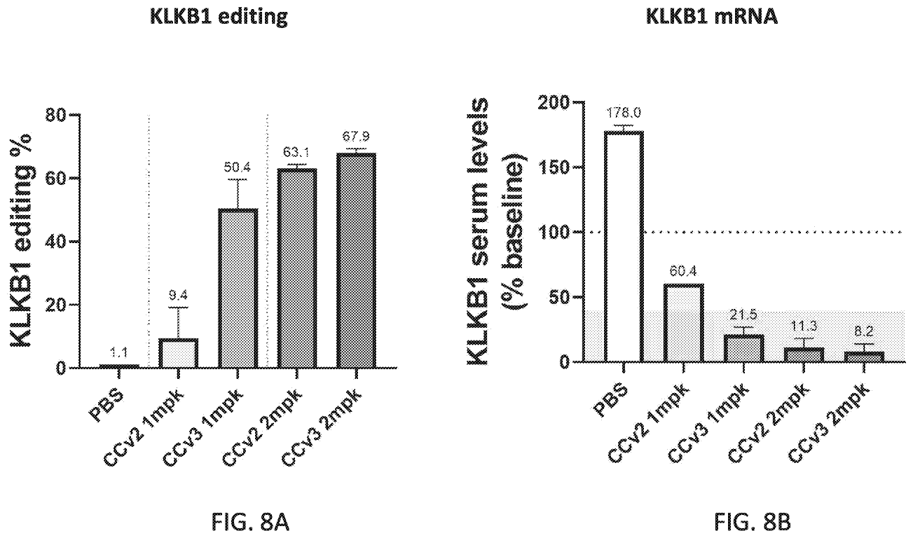

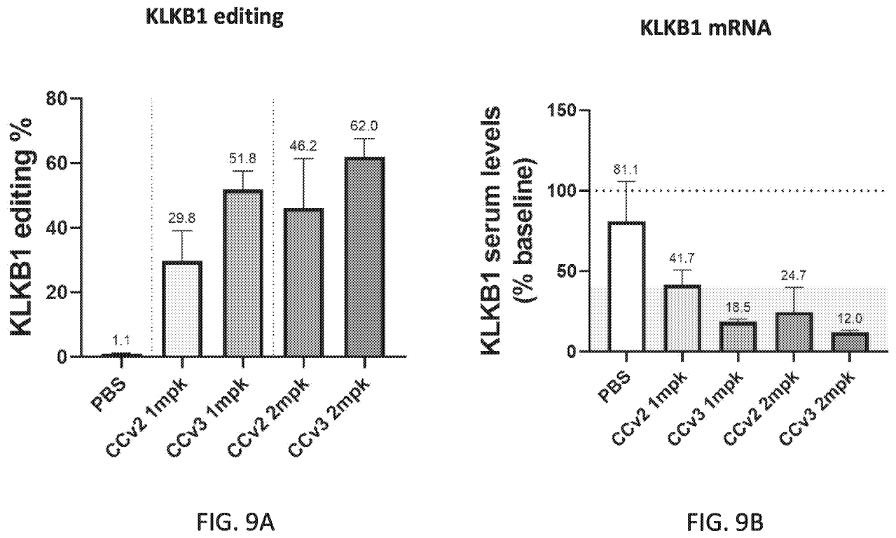

Adult male and female C57BL/6 mice were divided into five groups (n=3/group) and were intravenously administered: vehicle (control), 1 mg/kg of LNP Cas-CLOVER v2, 1 mg/kg LNP Cas-CLOVER v3, two administrations of 1 mg/kg of LNP Cas-CLOVER v2, or 2 mg/kg of LNP Cas-CLOVER v3. After seven days, mice were euthanized, liver tissue was resected, and genomic DNA from isolated liver tissue was isolated as described in Example 7. The percent indels (“% indels”) at the murine Klkb1 locus was determined by ddPCR for each of the Cas-CLOVER versions and doses for male and female mice (FIGS. 8A and 9A). In addition, Klkb1 mRNA levels were calculated for each LNP composition and administration to determine the percent decrease in Klkb1 mRNA levels compared in males and females to determine any gender differences. The results are shown in FIG. 8B, FIG. 9B, Table 7 (males), and Table 8 (females).

TABLE 7
Males

| Dose | % indels | Klkb1 mRNA serum level (% baseline) |
|---|---|---|
| 0 | 1.1 | 178.0 |
| 1 mg/kg CCv2 | 9.4 | 60.4 |
| 1 mg/kg CCv3 | 50.4 | 21.5 |
| 2 mg/kg CCv2 | 63.1 | 11.3 |
| 2 mg/kg CCv3 | 67.9 | 8.2 |

TABLE 8
Females

| Dose | % indels | Klkb1 mRNA serum level (% baseline) |
|---|---|---|
| 0 | 1.1 | 81.1 |
| 1 mg/kg CCv2 | 29.8 | 41.7 |
| 1 mg/kg CCv3 | 51.8 | 18.5 |
| 2 mg/kg CCv2 | 46.2 | 24.7 |
| 2 mg/kg CCv3 | 62.0 | 12.0 |

As shown in Table 7 and Table 8, both versions of Cas-CLOVER mRNA were capable of reducing KLKB1 in vivo protein serum levels in treated male and female mice, with Cas-CLOVER v3 demonstrating enhanced activity compared to Cas-CLOVER v2 at both doses.

### Example 9—LNP Compositions of Present Disclosure Deliver RNA with High Specificity to the Liver In Vivo

This experiment shows the ability of LNP compositions of the present disclosure to deliver Cas-CLOVER mRNA to the liver, targeted by a pair of gRNAs to the mouse KLKB1 gene, resulting in subsequent in vivo gene editing of the KLKB1 gene. The KLKB1 gene encodes for the protease kallikrein, which is elevated during attacks of hereditary angioedema, a rare genetic disorder characterized by recurrent episodes of the accumulation of fluids outside of the blood vessels, blocking the normal flow of blood or lymphatic fluid and causing rapid swelling of tissue in the hands, feet, limbs, face, intestinal tract or airways.

In this experiment, each group of adult female BALB/C mice (n=2/group) was intravenously co-administered mRNA encoding 5′-CleanCap-Cas-CLOVER v.3 (SEQ ID NO: 39) and a pair of gRNAs (SEQ ID NO: 53 and SEQ ID NO: 54) targeted to exon 11 of the mouse KLKB1 gene. The mRNA and gRNA molecules were formulated within LNP compositions of the present disclosure as shown in Table 9. All uridine residues in the mRNA were N1-methylpsuedouridine.

TABLE 9

| | COMPOUND | | | | |
|---|---|---|---|---|---|
| LNP | NO. 37 | phospholipid | Chol | PEG-DMG | Lipid:RNA |
| ID | (% moles) | (% moles) | (% moles) | (% moles) | (w/w) |
| 7.1 | 50 | 10% DSPC | 38.5 | 1.5 | 40 |
| 7.2 | 50 | 10% DSPC | 38.5 | 1.5 | 50 |
| 7.3 | 50 | 10% DPPC | 38.5 | 1.5 | 40 |
| 7.4 | 50 | 10% DPPC | 38.5 | 1.5 | 50 |
| 7.5 | 50 | 10% DSPC | 38 | 2 | 40 |
| 7.6 | 50 | 10% DSPC | 38 | 2 | 50 |
| 7.7 | 50 | 10% DPPC | 38 | 2 | 40 |
| 7.8 | 50 | 10% DPPC | 38 | 2 | 50 |

LNP compositions of the present disclosure (0.5 mg/kg or 1 mg/kg) were administered to the mice from each group. One group of mice was treated with vehicle (PBS) as a negative control.

Target site gene editing by the Cas-CLOVER mRNA delivered to the mice was measured by Droplet Digital PCR (ddPCR). Briefly, 50 ng of genomic DNA, primers and labeled probes were fractionated into 20000 droplets and PCR amplified with the following cycling parameters: 95 C for 10 min, 40 cycles of 94 C (30 sec), 55 C (1 min), and a final step of 98 C for 10 min. Then, samples were read with a QX200 droplet reader (Biorad) following manufacturer instructions. Percentage of indels was calculated as (# of NEHJ droplets×100)/(# of NHEJ+# of reference droplets). Results of the ddPCR are provided in Table 10 as indel percentages found at the KLKB1 intron 11 insertion site

TABLE 10

| | indel % | indel % |
|---|---|---|
| LNP ID | (0.5 mpk dose) | (1.0 mpk dose) |
| PBS control | 0 | 0 |
| 7.1 | 25.4 | 53.0 |
| 7.2 | 30.2 | 52.4 |
| 7.3 | 29.7 | 51.6 |
| 7.4 | 26.6 | 51.6 |
| 7.5 | 16.3 | 46.4 |
| 7.6 | 29.5 | 50.4 |
| 7.7 | 20.2 | 44.1 |
| 7.8 | 24.6 | 49.7 |

The results of this Example show that LNP compositions of the present disclosure successfully delivered Cas-CLOVER mRNA to the liver as shown by subsequent gene editing of the KLKB1 gene.

### Example 10—LNP Compositions of Present Disclosure Deliver RNA with High Specificity to the Liver In Vivo

As described in Example 9, LNP compositions of the present disclosure were used to deliver Cas-CLOVER mRNA to the liver, targeted by a pair of gRNAs to the mouse KLKB1 gene, resulting in subsequent in vivo gene editing of the KLKB1 gene.

In this experiment, each group of adult female BALB/C mice (n=2/group) was intravenously co-administered mRNA encoding 5′-CleanCap-Cas-CLOVER v.3 (SEQ ID NO: 39) and a pair of gRNAs (SEQ ID NO: 53 and SEQ ID NO: 54) targeted to exon 11 of the mouse KLKB1 gene. The mRNA and gRNA molecules were formulated within LNP compositions of the present disclosure comprising COMPOUND NO. 37, DSPC, Cholesterol and DMG-PEG2000 at the following molar ratio: 50:10:38.5:1.5, with a lipid:nucleic acid ratio of 50:1. All uridine residues in the mRNA were N1-methylpsuedouridine.

LNP compositions of the present disclosure were administered to the mice from each group at the following doses: 0.125 mg/kg, 0.25 mg/kg, 0.5 mg/kg, 1 mg/kg, 2 mg/kg, and 3 mg/kg. One group of mice was treated with vehicle (PBS) as a negative control.

After seven days post-administration, DNA was isolated from four tissue types from the mice in each group: liver, spleen, lung, and kidney. Briefly, tissues were resected after euthanasia, flash frozen in liquid nitrogen, mixed with lysis buffer (15 mg of tissue in 200 μL of lysis buffer+10 μL Proteinase K) and pulverized in a TissueLyser II (Qiagen) using Triple-Pure zirconium beads. Homogenized tissue was then incubated at 56 C for 30 minutes, and column-purified using a Monarch Genomic DNA Purification kit from New England Biolabs under the manufacturer's instructions. Final DNA elution was done in 50 μL of elution buffer (10 mM Tris-Cl, pH 8.5). The concentration and purity of DNA samples were assessed by measuring absorbance at 260 and 280 nm using a Nanodrop. Also, blood samples were drawn for LDL-C quantification. Briefly, 500 μL of blood was collected after euthanasia via cardiac puncture using 2 ml syringes and 25 G needles, transferred to microcentrifuge tubes, incubated at room temperature for 1 hour, and centrifuged at 1500 g for 15 minutes to separate the cellular fraction from serum. Serum fraction (200 μL) was transferred to a new tube and stored at −80 C until further analysis.

Serum levels of the kallikrein in the mice were measured 7 days after administration and the results are shown in Table 11. Briefly, a mouse ELISA kit (Biolegend) was used to determine kallikrein in each serum sample following the manufacturer's instructions. All serum samples were assayed in triplicate and results were expressed as the percentage reduction in kallikrein levels compared with kallikrein levels of PBS-treated mice.

TABLE 11

| LNP dose (mg/kg) | % kallikrein relative to baseline |
|---|---|
| 0.125 | 99.55 |
| 0.25 | 90.9 |
| 0.5 | 55.8 |
| 1 | 10.3 |
| 2 | 7.3 |
| 3 | 4.75 |

Additionally, as described in Example 9, target site gene editing by the Cas-CLOVER mRNA delivered to the mice was measured by Droplet Digital PCR (ddPCR). Results of the ddPCR are provided in Table 12 as indel percentages found at the KLKB1 intron 11 insertion site.

TABLE 12

| LNP dose (mg/kg) | indel % |
|---|---|
| 0.125 | 7.78 |
| 0.25 | 9.12 |
| 0.5 | 36.74 |
| 1 | 63.31 |
| 2 | 68.13 |
| 3 | 72.96 |
| vehicle | 0 |

The results of this Example show that LNP compositions of the present disclosure successfully delivered Cas-CLOVER mRNA to the liver as shown by subsequent gene editing of the KLKB1 gene and reduction in plasma kallikrein protein levels in treated mice, both which were also shown to be dose-dependent in this Example.

### Example 11—LNP Compositions of Present Disclosure Deliver RNA with High Specificity to the Liver In Vivo

As described in Example 9, LNP compositions of the present disclosure were used to deliver Cas-CLOVER mRNA to the liver, targeted by a pair of gRNAs to the mouse KLKB1 gene, resulting in subsequent in vivo gene editing of the KLKB1 gene.

In this experiment, each group of adult female BALB/C mice (n=2/group) was intravenously co-administered mRNA encoding 5′-CleanCap-Cas-CLOVER v.3 (SEQ ID NO: 39) and a pair of gRNAs (SEQ ID NO: 53 and SEQ ID NO: 54) targeted to exon 11 of the mouse KLKB1 gene. The mRNA and gRNA molecules were formulated within LNP compositions of the present disclosure comprising COMPOUND NO. 37, DSPC, Cholesterol and DMG-PEG2000 at the following molar ratio: 50:10:38:2, with a lipid:nucleic acid ratio of 50:1. All uridine residues in the mRNA were N1-methylpsuedouridine.

LNP compositions of the present disclosure were administered to the mice from each group at the following doses: 0.125 mg/kg, 0.25 mg/kg, 0.5 mg/kg, 1 mg/kg, 2 mg/kg, and 3 mg/kg. One group of mice was treated with vehicle (PBS, Thermo Fisher Scientific, USA) as a negative control.

As described in Example 10, serum levels of the kallikrein in the mice were measured 7 days after administration and the results are shown in Table 13.

TABLE 13

| LNP dose (mg/kg) | % kallikrein relative to baseline |
|---|---|
| 0.125 | 97.8 |
| 0.25 | 53.2 |
| 0.5 | 34.6 |
| 1 | 21.0 |
| 2 | 4.6 |
| 3 | 3.3 |

Additionally, as described in Example 9, target site gene editing by the Cas-CLOVER mRNA delivered to the mice was measured by Droplet Digital PCR (ddPCR). Results of the ddPCR are provided in Table 14 as indel percentages found at the KLKB1 intron 11 insertion site.

TABLE 14

| LNP dose (mg/kg | indel % |
|---|---|
| 0.125 | 12.3 |
| 0.25 | 30.5 |
| 0.5 | 57.8 |
| 1 | 66.5 |
| 2 | 69.8 |
| 3 | 71.6 |
| vehicle | 0 |

The results of this Example show that LNP compositions of the present disclosure successfully delivered Cas-CLOVER mRNA to the liver as shown by subsequent gene editing of the KLKB1 gene and reduction in plasma kallikrein protein levels in treated mice, both of which were also shown to be dose-dependent in this Example.

### Example 12—LNP Compositions of Present Disclosure Deliver RNA with High Specificity to the Liver In Vivo

This experiment shows the ability of LNP compositions of the present disclosure to deliver Cas-CLOVER mRNA to the liver, targeted by a pair of gRNAs to the mouse KLKB1 gene, resulting in subsequent in vivo gene editing of the KLKB1 gene. The KLKB1 gene encodes for the protease kallikrein, which is elevated during attacks of hereditary angioedema, a rare genetic disorder characterized by recurrent episodes of the accumulation of fluids outside of the blood vessels, blocking the normal flow of blood or lymphatic fluid and causing rapid swelling of tissue in the hands, feet, limbs, face, intestinal tract or airways.

In this experiment, each group of adult female BALB/C mice (n=2/group) was intravenously co-administered mRNA encoding 5′-CleanCapM6-Cas-CLOVER v.3 (SEQ ID NO: 39) and a pair of gRNAs (SEQ ID NO: 53 and SEQ ID NO: 54) targeted to exon 11 of the mouse KLKB1 gene. The mRNA and gRNA molecules were formulated within LNP compositions of the present disclosure as shown in Table 15. All uridine residues in the mRNA were N1-methylpsuedouridine.

TABLE 15

| | COMPOUND | DOPC | Chol | PEG- | Lipid:Nucleic |
|---|---|---|---|---|---|
| LNP | NO. 1 | (% | (% | DMG | Acid |
| ID | (% moles) | moles) | moles) | (% moles) | (w/w) |
| 3.1 | 40 | 10 | 48.5 | 1.5 | 60 |
| 3.2 | 40 | 10 | 48.5 | 1.5 | 50 |
| 3.3 | 40 | 10 | 48.5 | 1.5 | 40 |
| 3.4 | 40 | 10 | 48.5 | 1.5 | 30 |
| 3.5 | 50 | 10 | 38.5 | 1.5 | 60 |
| 3.6 | 50 | 10 | 38.5 | 1.5 | 50 |
| 3.7 | 50 | 10 | 38.5 | 1.5 | 40 |

LNP compositions of the present disclosure (0.5 mg/kg) were administered to the mice from each group. One group of mice was treated with vehicle (PBS) as a negative control.

Target site gene editing by the Cas-CLOVER mRNA delivered to the mice was measured by Droplet Digital PCR (ddPCR). Briefly, 50 ng of genomic DNA, primers and labeled probes were fractionated into 20000 droplets and PCR amplified with the following cycling parameters: 95 C for 10 min, 40 cycles of 94 C (30 sec), 55 C (1 min), and a final step of 98 C for 10 min. Then, samples were read with a QX200 droplet reader (Biorad) following manufacturer instructions. Percentage of indels was calculated as (# of NEHJ droplets×100)/(# of NHEJ+ # of reference droplets). Results of the ddPCR are provided in Table 16 as indel percentages found at the KLKB1 intron 11 insertion site.

TABLE 16

| LNP ID | indel % |
|---|---|
| 3.1 | 46.8 |
| 3.2 | 45.8 |
| 3.3 | 28.4 |
| 3.4 | 33.0 |
| 3.5 | 36.2 |
| 3.6 | 53.5 |
| 3.7 | 48.0 |
| vehicle | 0 |

As shown in Table 16, LNP compositions of the present disclosure with varying lipid:nucleic acid ratios successfully delivered Cas-CLOVER mRNA to the liver as shown by subsequent gene editing of the KLKB1 gene, with a range of activities from about 28% to greater than 50% edited KLKB1 loci.

### Example 13—LNP Compositions of Present Disclosure Deliver RNA with High Specificity to the Liver In Vivo

As described in Example 12, LNP compositions of the present disclosure were used to deliver Cas-CLOVER mRNA to the liver, targeted by a pair of gRNAs to the mouse KLKB1 gene, resulting in subsequent in vivo gene editing of the KLKB1 gene.

In this experiment, each group of adult female BALB/C mice (n=2/group) was intravenously co-administered mRNA encoding 5′-CleanCapM6-Cas-CLOVER v.3 (SEQ ID NO: 39) and a pair of gRNAs (SEQ ID NOs: 53 and 54) targeted to exon 11 of the mouse KLKB1 gene. The mRNA and gRNA molecules were formulated within LNP compositions of the present disclosure comprising COMPOUND NO. 1, DOPC, Cholesterol and DMG-PEG2000 at the molar ratios shown in Table 17. All uridine residues in the mRNA were N1-methylpsuedouridine.

TABLE 17

| | COMPOUND | DOPC | Chol | PEG- | Lipid:Nucleic |
|---|---|---|---|---|---|
| LNP | NO. 1 | (% | (% | DMG | Acid |
| ID | (% moles) | moles) | moles) | (% moles) | (w/w) |
| 3-5 | 50 | 10 | 38.5 | 1.5 | 60 |
| 1-15 | 50 | 10 | 38.5 | 1.5 | 80 |
| 3-8 | 45 | 10 | 43 | 2 | 60 |
| 2-1 | 45 | 10 | 43 | 2 | 80 |
| 3-1 | 40 | 10 | 48.5 | 1.5 | 60 |
| 2-10 | 40 | 10 | 48.5 | 1.5 | 80 |

LNP compositions of the present disclosure (0.5 mg/kg or 1 mg/kg) were administered to the mice from each group. One group of mice was treated with vehicle (PBS, Thermo Fisher Scientific, USA) as a negative control.

As described in Example 12, target site gene editing by the Cas-CLOVER mRNA delivered to the mice was measured by Droplet Digital PCR (ddPCR). Results of the ddPCR are provided in Table 18 as indel percentages found at the KLKB1 intron 11 insertion site.

TABLE 18

| LNP ID | indel % (0.5 mpk dose) | indel % (1.0 mpk dose) |
|---|---|---|
| 3-5 | 29.8 | 58.3 |
| 1-15 | 48.6 | 60.8 |
| 3-8 | 30.7 | 55.1 |
| 2-1 | 43.3 | 58.3 |
| 3-1 | 43.7 | 58.7 |
| 2-10 | 49.0 | 54.1 |
| vehicle | 0 | 0 |

As shown in Table 18, LNP compositions of the present disclosure successfully delivered Cas-CLOVER mRNA to the liver as shown by subsequent gene editing of the KLKB1 gene.

### Example 14—LNP Compositions of Present Disclosure Deliver RNA with High Specificity to the Liver In Vivo

As described in Example 12, LNP compositions of the present disclosure were used to deliver Cas-CLOVER mRNA to the liver, targeted by a pair of gRNAs to the mouse KLKB1 gene, resulting in subsequent in vivo gene editing of the KLKB1 gene.

In this experiment, each group of adult female BALB/C mice (n=2/group) was intravenously co-administered mRNA encoding 5′-CleanCap-Cas-CLOVER v.3 (SEQ ID NO: 39) and a pair of gRNAs (SEQ ID NOs: 53 and 54) targeted to exon 11 of the mouse KLKB1 gene. The mRNA and gRNA molecules were formulated within LNP compositions of the present disclosure comprising COMPOUND NO. 1, DOPC, Cholesterol and DMG-PEG2000 at the following molar ratio: 40:10:48.5:1.5, with a lipid:nucleic acid ratio of 60:1. All uridine residues in the mRNA were N1-methylpsuedouridine.

LNP compositions of the present disclosure were administered to the mice from each group at the following doses: 0.125 mg/kg, 0.25 mg/kg, 0.5 mg/kg, 1 mg/kg, 2 mg/kg, and 3 mg/kg. One group of mice was treated with vehicle (PBS, Thermo Fisher Scientific, USA) as a negative control.

After seven days post-administration, DNA was isolated from four tissue types from the mice in each group: liver, spleen, lung, and kidney. Briefly, tissues were resected after euthanasia, flash frozen in liquid nitrogen, mixed with lysis buffer (15 mg of tissue in 200 μL of lysis buffer+10 μL Proteinase K) and pulverized in a TissueLyser II (Qiagen) using Triple-Pure zirconium beads (Fisher Scientific). Homogenized tissue was then incubated at 56 C for 30 minutes, and column-purified using a Monarch Genomic DNA Purification kit from New England Biolabs under manufacturer's instructions. Final DNA elution was done in 50 μL of elution buffer (10 mM Tris-Cl, pH 8.5). The concentration and purity of DNA samples were assessed by measuring absorbance at 260 and 280 nm using a Nanodrop. Also, blood samples were drawn for LDL-C quantification. Briefly, 500 μL of blood was collected after euthanasia via cardiac puncture using 2 ml syringes and 25 G needles, transferred to microcentrifuge tubes, incubated at room temperature for 1 hour, and centrifuged at 1500 g for 15 minutes to separate the cellular fraction from serum. Serum fraction (200 μL) was transferred to a new tube and stored at −80 C until further analysis.

Serum levels of the kallikrein in the mice were measured 7 days after administration and the results are shown in Table 19. Briefly, a mouse ELISA kit (Biolegend) was used to determine kallikrein in each serum sample following manufacturer's instructions. All serum samples were assayed in triplicate and results were expressed as the percentage reduction in kallikrein levels compared with kallikrein levels of PBS-treated mice.

TABLE 19

| LNP dose (mg/kg) | % kallikrein relative to baseline |
|---|---|
| 0.125 | 101.5 |
| 0.25 | 112 |
| 0.5 | 30.1 |
| 1 | 4.75 |
| 2 | 3.7 |
| 3 | 2.2 |

As described in Example 12, target site gene editing by the Cas-CLOVER mRNA delivered to the mice was measured by Droplet Digital PCR (ddPCR). Results of the ddPCR are provided in Table 20 as indel percentages found at the KLKB1 intron 11 insertion site.

TABLE 20

| LNP dose (mg/kg) | indel % |
|---|---|
| 0.125 | 0.77 |
| 0.25 | 11.25 |
| 0.5 | 44.21 |
| 1 | 65.13 |
| 2 | 71.58 |
| 3 | 69.13 |
| vehicle | 0 |

The results of this Example show that LNP compositions of the present disclosure successfully delivered Cas-CLOVER mRNA to the liver as shown by subsequent gene editing of the KLKB1 gene and reduction in plasma kallikrein protein levels in treated mice, both of which were also shown to be dose-dependent in this Example.

### Example 15—LNP Compositions of Present Disclosure Deliver RNA with High Specificity to the Liver In Vivo

As described in Example 12, LNP compositions of the present disclosure were used to deliver Cas-CLOVER mRNA to the liver, targeted by a pair of gRNAs to the mouse KLKB1 gene, resulting in subsequent in vivo gene editing of the KLKB1 gene.

In this experiment, each group of adult female BALB/C mice (n=2/group) was intravenously co-administered mRNA encoding 5′-CleanCap-Cas-CLOVER v.3 (SEQ ID NO: 39) and a pair of gRNAs (SEQ ID NOs: 53 and 54) targeted to exon 11 of the mouse KLKB1 gene. The mRNA and gRNA molecules were formulated within LNP compositions of the present disclosure comprising COMPOUND NO. 1, DOPC, Cholesterol and DMG-PEG2000 at the following molar ratio: 40:10:48.5:1.5, with a lipid:nucleic acid ratio of 50:1. All uridine residues in the mRNA were N1-methylpsuedouridine.

LNP compositions of the present disclosure were administered to the mice from each group at the following doses: 0.125 mg/kg, 0.25 mg/kg, 0.5 mg/kg, 1 mg/kg, 2 mg/kg, and 3 mg/kg. One group of mice was treated with vehicle (PBS, Thermo Fisher Scientific, USA) as a negative control.

As described in Example 14, serum levels of the kallikrein in the mice were measured 7 days after administration and the results are shown in Table 21.

TABLE 21

| LNP dose (mg/kg) | % kallikrein relative to baseline |
|---|---|
| 0.125 | 151.3 |
| 0.25 | 125.9 |
| 0.5 | 30.0 |
| 1 | 4.9 |
| 2 | 1.6 |
| 3 | 1.7 |

Additionally, as described in Example 12, target site gene editing by the Cas-CLOVER mRNA delivered to the mice was measured by Droplet Digital PCR (ddPCR). Results of the ddPCR are provided in Table 22 as indel percentages found at the KLKB1 intron 11 insertion site.

TABLE 22

| LNP dose (mg/kg) | indel % |
|---|---|
| 0.125 | 0.8 |
| 0.25 | 7.9 |
| 0.5 | 45.1 |
| 1 | 63.4 |
| 2 | 68.0 |
| 3 | 71.8 |
| vehicle | 0 |

The results of this Example show that LNP compositions of the present disclosure successfully delivered Cas-CLOVER mRNA to the liver as shown by subsequent gene editing of the KLKB1 gene and reduction in plasma kallikrein protein levels in treated mice, both which were also shown to be dose-dependent in this Example.

### Example 16: Generation of Partially Liver Humanized CB57BL/6 Mice by Germline Editing of the Murine KLKB1 Locus to a Humanized KLKB1 Locus

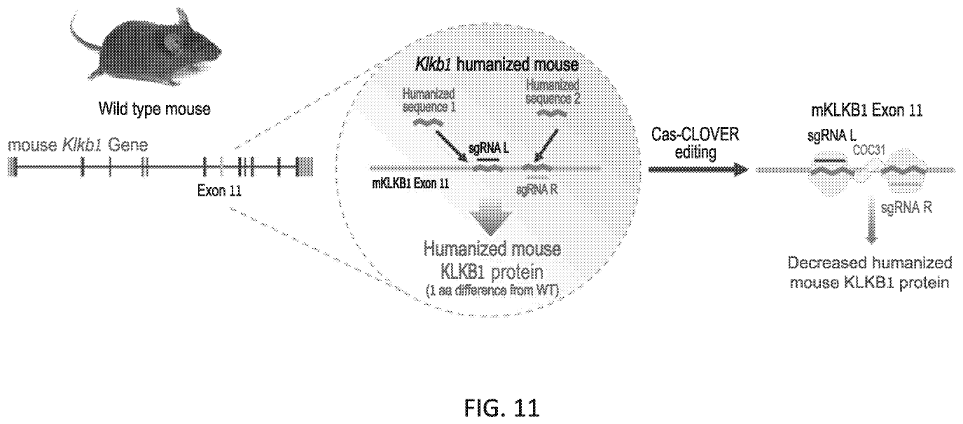

A partially humanized KLKB1 mouse model (huKLKB1 mice) was generated for the efficacy evaluation of P-KLKB1-101 (FIG. 11). Briefly, the partially humanized mouse model was generated by germline introduction of the human sgRNA target sequences into the mKlkb1 gene to produce P-KLKB1-101-editable loci in all somatic and reproductive cells.

Genetic humanization of Exon 11 of the mKlkb1 locus was targeted at the exact corresponding site where hKLKB1 sgRNAs (gRNA Pair #16) recognize and guide cleavage of the Cas-CLOVER nuclease. A total of 5 nucleotides in the mouse genome were changed to allow 100% match to the sgRNA protospacer sequences, with 2 additional base pairs modified to match the human protospacer adjacent motif (PAM). These sequence changes result in a single amino acidic change in the translated protein and complete preservation of the mouse regulatory elements that control Klkb1 expression. This humanization enables human-specific P-KLKB1-101 to recognize and edit this surrogate target site. In the homozygous state of this humanized allele, editing of the target site would cause a reduction in kallikrein protein expression and plasma levels, per the same designed mechanism as intended in humans.

### Example 17: Editing of Human KLKB1 Locus in Liver Humanized Mice

LNP compositions comprising 50% COMPOUND NO. 37 by moles; 38.5% Cholesterol by moles, 10% DSPC by moles and 1.5% DMG-PEG by moles, the gRNA Pair #16 targeting human KLKB1 locus, and an mRNA encoding Cas-CLOVER v3.0 (SEQ ID NO: 39) were prepared for delivering editing composition to the liver of mice.

The huKLKB1 liver humanized mice of Example 16 were divided into six groups (n=3/group). On Day-1, body weights were measured, whole blood samples were collected for baseline cytokine and liver enzyme levels, and health assessment scores (HAS) were made for each mouse. On Day 0, mice were intravenously administered one of: vehicle (control), 0.125 mg/kg, 0.25 mg/kg, 0.5 mg/kg, 1.0 mg/kg, or 3.0 mg/kg of LNP Cas-CLOVERv3. After 24 hours, whole blood samples were collected, HAS scores determined, and body weights determined.

After seven days, mice were euthanized, and a terminal blood sample was collected for determining post-administration cytokine and liver enzyme levels, and for calculating serum kallikrein levels. Liver tissue was resected, and flash frozen in liquid nitrogen. To isolate genomic DNA, tissue samples were mixed with lysis buffer (15 mg of tissue in 200 μL of lysis buffer+10 L Proteinase K) and pulverized in a TissueLyser II using Triple-Pure zirconium beads. Homogenized tissue was then incubated at 56° C. for 30 minutes and column-purified using a Monarch® Genomic DNA Purification kit in accordance with manufacturer's instructions. Final DNA elution was performed in 50 μL of elution buffer (10 mM Tris-Cl, pH 8.5). The concentration and purity of DNA samples were assessed by measuring absorbance at 260 and 280 nm using a NanoDrop® device. DNA samples were used for KLKB1 editing quantification determined by amplicon-seq for control and treated mice as described in Example 3. The results are shown in Table 23.

TABLE 23

| Dose | % Indels |
|---|---|
| 0 | 0.01 |
| 0.125 mg/kg Cas-CLOVER LNP | 17.9 |
| 0.25 mg/kg Cas-CLOVER LNP | 45.6 |
| 0.5 mg/kg Cas-CLOVER LNP | 53.8 |
| 1.0 mg/kg Cas-CLOVER LNP | 63.7 |
| 3.0 mg/kg Cas-CLOVER LNP | 62.8 |

As shown in Table 23, Cas-CLOVER v3 editing resulted in a dose-dependent increase in KLKB1 editing levels with editing levels peaking at 1.0 mg/kg demonstrating that the human KLKB1 targeting construct can successfully edit the huKLKB1 locus in liver humanized mice.

In addition, plasma kallikrein levels were calculated for each LNP administration using an ELISA assay (Boster Bio, Pleasanton, CA) to determine the percent reduction in plasma kallikrein levels in treated versus untreated controls. The results are shown in Table 24.

TABLE 24

| Dose | % Plasma Kallikrein Levels |
|---|---|
| 0 | 95.33 |
| 0.125 mg/kg Cas-CLOVER LNP | 42.07 |
| 0.25 mg/kg Cas-CLOVER LNP | 6.47 |
| 0.5 mg/kg Cas-CLOVER LNP | 0.00 |
| 1.0 mg/kg Cas-CLOVER LNP | 0.00 |
| 3.0 mg/kg Cas-CLOVER LNP | 0.00 |

As shown in Table 24, Cas-CLOVER v2 editing robustly reduces plasma kallikrein levels in treated liver humanized mice in a dose-dependent manner with LNP concentrations of at least 0.5 mg/kg of KLKB1 LNP resulting in plasma kallikrein levels below the limits of detection for the assay.

Furthermore, plasma kallikrein levels also were determined for control and LNP administered huKLKB1 mice by measuring plasma kallikrein-mediated cleavage of high molecular weight kininogen (HMWK) by Western blotting. Briefly, plasma samples obtained from control and LNP administered mice were incubated with 15 μg/mL of Kaolin (Sigma-Aldritch, St Louis, MO) in 96 well plates at 37° C. for 3 minutes. Samples were then treated with RIPA lysis buffer, boiled at 95° C. for 5 minutes, and a portion of each sample (20 ug or ug/ml) was subjected to denaturing acrylamide gel electrophoresis to separate substrate and cleavage products. The electrophoretically separated proteins were transferred to a PVDF membrane. The protein-bound membrane was incubated with 1 μg/mL of an anti-HMWK antibody (R&D Systems, Minneapolis, MN) to detect the substrate and cleaved products and the bound antibody was visualized by incubating with an HRP-labeled anti-rat IgG antibody (R&D Systems). The amount of HMWK substrate and cleaved products was determined by quantitative densitometry.

In addition, plasma levels of bradykinin are regulated by plasma kallikrein-mediated cleavage of high molecular weight kininogen (HMWK). The levels of plasma bradykinin were determined for control and LNP administered huKLKB1 mice using an ELISA (Phoenix Pharmaceuticals, Burlingame, CA) in accordance with the manufacturer's instructions to determine the percent reduction from baseline of plasma bradykinin levels. The percent of cleaved HMWK from the Western blot and the plasma levels of bradykinin as a percent baseline were determined. The results are shown in Table 25.

TABLE 25

| | Plasma Kallikrein | Plasma Bradykinin |
|---|---|---|
| Dose | Activity (% Vehicle) | Levels (% Baseline) |
| 0 | 100.00 | 134.63 |
| 0.125 mg/kg LNP | 85.30 | 62.87 |
| 0.25 mg/kg LNP | 9.27 | 35.45 |
| 0.5 mg/kg LNP | 6.40 | 18.12 |
| 1.0 mg/kg LNP | 1.06 | 5.34 |
| 3.0 mg/kg LNP | 2.67 | 2.27 |

As shown in Table 25, liver humanized mice treated with KLKB1 LNPs exhibited a dose-dependent reduction in plasma kallikrein activity levels measured as a percentage of HMWK cleavage and exhibited a dose-dependent decrease in plasma bradykinin mice compared to untreated controls. Without wishing to be bound by theory, these results demonstrate that editing of the KLKB1 locus of liver humanized mice reduces plasma kallikrein levels and a concomitant reduction in plasma bradykinin levels in treated animals due to decreased plasma kallikrein activity in these mice.

### Example 18: Exploratory Safety of mRNA+gRNA LNP Candidates Targeting Kallikrein B1 (KLKB1) Following Single Intravenous (IV) Administration in African Green Monkeys

Dose escalation of LNP compositions comprising the gRNA Pair #16 targeting the human KLKB1 locus, and an mRNA encoding Cas-CLOVER v3.0 (SEQ ID NO: 39) were prepared for delivering editing composition to African green monkeys.

TABLE 26
Formulations

| Formulation | | | | Lipid Ratios | mRNA: | Lipid: |
|---|---|---|---|---|---|---|
| # | LNP ID | Process | Lipids | (% moles) | gRNA | Payload |
| 1 | H2-8 | Benchtop | Compound | 50:10:38:2 | 2:1 | 50:1 |
| | RCD | process: PNI | No. 37: | | | |
| | | Ignite followed | DSPC: | | | |
| | | by dialysis and | Chol: | | | |
| | | centrifugation | DMG- | | | |
| | | | PEG2k | | | |
| 2 | H2-8 | Scalable process: | Compound | 50:10:38:2 | 2:1 | 50:1 |
| | ePD | scalable mixing | No. 37: | | | |
| | | device followed | DSPC: | | | |
| | | by tangential | Chol: | | | |
| | | flow filtration | DMG- | | | |
| | | and purification | PEG2k | | | |
| 3 | H2-8 | Scalable process: | Compound | 50:10:38:2 | 2:1 | 50:1 |
| | ePDv02 | scalable mixing | No. 37: | | | |
| | | device followed | DSPC: | | | |
| | | by tangential | Chol: | | | |
| | | flow filtration, | DMG- | | | |
| | | purification and | PEG2k | | | |
| | | minimization of | | | | |
| | | impurities | | | | |
| 4 | 3S-2 | Scalable process: | Compound | 40:10:48.5:1.5 | 2:1 | 50:1 |
| | | scalable mixing | No. 1: | | | |
| | | device followed | DOPC: | | | |
| | | by tangential | Chol: | | | |
| | | flow filtration | DMG- | | | |
| | | and purification | PEG2k | | | |
| 5 | H7-7 | Scalable process: | Compound | 50:5:42:3 | 2:1 | 50:1 |
| | | scalable mixing | No. 37: | | | |
| | | device followed | DSPC: | | | |
| | | by tangential | Chol: | | | |
| | | flow filtration | DMG- | | | |
| | | and purification | PEG2k | | | |

### Study Design

Subject Recruitment: Screening physical exams and clinical pathology evaluations were conducted. Monkeys (male, “M” and female, “F”) with normal findings were recruited to the study and assigned to a treatment group (Table 27). For baseline screening and all subsequent procedures, anesthesia will be achieved with intramuscular ketamine (8 mg/kg) and xylazine (1.6 mg/kg) to effect.

TABLE 27
Treatment Assignment

| | Number of | | | Conc. | Volume | Dose | Dose | | Test and Control |
|---|---|---|---|---|---|---|---|---|---|
| Group | subjects | Treatment | Route | (mg/mL) | (mL/kg) | (mg/kg) | Interval | Sacrifice | Article Required |
| 1 | 1 | Vehicle B | IV | 1 | 2.5to 5 | N/A | Day 0 | Day 45 | (5 mL/kg × 6 kg × |
| | (1F) | | | | | | | | 20% overage) = |
| | | | | | | | | | 36 mL |
| 2 | 2 | Formulation 1 | IV | 1 | 2.5 to 5 | 0.75 | Day 0 | Day 30 | (5 mL/kg × 6 kg × |
| | (1M/1F) | | | | | mg/kg | | | 20% overage) × |
| | | | | | | | | | 2 = 72 mL |
| | 2 | Formulation 2 | IV | 1 | 2.5 to 5 | 2 | Day 14 | Day 59 | (5 mL/kg × 6 kg × |
| | (1M/1F) | | | | | mg/kg | | | 20% overage) × |
| | | | | | | | | | 2 = 72 mL |
| | 1 | Formulation 4 | IV | 1 | 2.5 to 5 | 0.25 | Day 14 | Day 59 | (5 mL/kg × 6 kg × |
| | (1F) | | | | | mg/kg | | | 20% overage) = |
| | | | | | | | | | 36 mL |
| 5 | 1 | Vehicle A | IV | 1 | 2.5 to 5 | N/A | Day 7 | Day 92 | (5 mL/kg × 6 kg × |
| | (1F) | | | | | | | | 20% overage) = |
| | | | | | | | | | 36 mL |
| 6 | 2 | Formulation 2 | IV | 1 | 2.5 to 5 | 1 | Day 7 | Day 52 | (5 mL/kg × 6 kg × |
| | (1M/1F) | | | | | mg/kg | | | 20% overage) × |
| | | | | | | | | | 2 = 72 mL |
| 7 | 2 | Formulation 2 | IV | 1 | 2.5 to 5 | 0.25 | Day 21 | Day 66 | (5 mL/kg × 6 kg × |
| | (1M/1F) | | | | | mg/kg | | | 20% overage) × |
| | | | | | | | | | 2 = 72 mL |
| 8 | 1 | Formulation 4 | IV | 1 | 2.5 to 5 | 1 | Day 21 | Day 66 | (5 m.L/kg × 6 kg × |
| | (1F) | | | | | mg/kg | | | 20% overage) = |
| | | | | | | | | | 36 mL |
| 9 | 1 | Formulation 2 | IV | 1 | 2.5 to 5 | 3 | Day 42 | Day 92 | (5 mL/kg × 6 kg × |
| | (1F) | | | | | mg/kg | | | 20% overage) × |
| | | | | | | | | | 2 = 72 mL |
| 10 | 2 | Formulation 3 | IV | 1 | 2.5 to 5 | 2 | ~Day 120 | ~Day 141 | (5 mL/kg × 6 kg × |
| | (2F)** | | | | | mg/kg | | | 20% overage) × |
| | | | | | | | | | 2 = 72 mL |
| 11 | 2 | Formulation 3 | IV | 1 | 2.5 to 5 | 3 | ~Day 166 | ~Day 187 | (5 mL/kg × 6 kg × |
| | (2F) | | | | | mg/kg | | | 20% overage) × |
| | | | | | | | | | 2 = 72 mL |
| 12 | 2 | Formulation 5 | IV | 1 | 2.5 to 5 | 3 | ~Day 166 | ~Day 187 | (5 mL/kg × 6 kg × |
| | (2F) | | | | | mg/kg | | | 20% overage) × |
| | | | | | | | | | 2 = 72 mL |
| 13 | 1 | Vehicle | IV | N/A | 2.5 to 5 | N/A | ~Day 166 | ~Day 187 | (5 mL/kg × 6 kg × |
| | (F) | | | | | | | | 20% overage) × |
| | | | | | | | | | 2 = 72 mL |

* Treatments will be staggered by 1 week or greater to monitor tolerance before escalating candidate and dose. Specifically, one animal from each test article treatment group will be dosed prior treatment of another animal.
**8 female (F) animals will be screened with body weights <3.8 kg

Intravenous Dosing: Animals are dosed in accordance with the treatment assignment (Table 27) based on dose day body weights. Each of Formulations 1-5 comprises an LNP composition comprising the gRNA Pair #16 targeting the human KLKB1 locus, and an mRNA encoding Cas-CLOVER v3.0 (SEQ ID NO: 39). Dosing animals were sedated with ketamine (1.6 mg/kg IM), administered atropine sulfate (0.02-0.04 mg/kg IM), intubated, placed in a prone position, and maintained on isoflurane anesthesia. Each monkey received an IV infusion via a saphenous vein (or alternative vein) after aseptic preparation with 70% isopropyl alcohol, or equivalent using a calibrated Harvard Apparatus Pump 11 Elite syringe pump (or equivalent) attached to a 20-gauge intravenous catheter (or equivalent infusion system) at a target rate of 2.5 to 5/mL/kg/hr over approximately 60 minutes to 150 minutes, with a saline flush. The actual start and stop times of dosing and vein used were recorded.

Immunosuppression: Animals received diphenhydramine (5 mg/kg intramuscular (IM)) three times daily from Day-1 through Day 1. Animals also received methylprednisolone (8 mg/kg IM) weekly, or another equivalent immunosuppression regimen.

TABLE 28
Groups 1, 2, 3, 4, 6, 7, and 8 Study Schedule (n = 11)

| | | Study Interval^{D} | | | | | | | | | | |
|---|---|---|---|---|---|---|---|---|---|---|---|---|
| | | | | Day 0 | | | | | | | | |
| | | Base- | Day | | 1 | 4 | Day | Day | Day | Day | Day | Day |
| Event | # | line | −1 | 0 | hour^{T} | hours^{T} | 1 | 3 | 7 | 14 | 21 | 30 |
| Dosing | 11 | — | — | X | — | — | — | — | — | — | — | — |
| Methylprednisolone* | 11 | — | X | — | — | — | X | — | X | — | — | — |
| Diphenhydramine** | 11 | — | X | X | — | — | X | — | — | — | — | — |
| Body weights | 11 | X | — | X | — | — | X | — | X | — | — | X |
| Physiology-ECG, RR, O₂ | 11 | X | — | — | — | — | X | — | X | — | — | X |
| Physical exam | 11 | X | — | — | — | — | X | — | X | — | — | X |
| Clinical chemistry | 11 | X | — | — | — | — | X | X | X | — | — | X |
| Complete blood count | 11 | X | — | — | — | — | X | X | X | — | — | X |
| Coagulation profile | 11 | X | — | — | — | — | X | X | X | — | — | X |
| Biomarker plasma | 11 | X | — | — | X | X | X | X | X | — | — | X |
| Serum | 11 | X | — | — | X | X | X | X | X | — | — | X |
| Semen collection | 11 | — | — | — | — | — | — | — | M | — | — | — |
| Necropsy and tissues | 11 | — | — | — | — | — | — | — | — | — | — | X |

X = event for animals in each dosing group with respect to respective Day 0 dosing day.
M = event for all males
^{T}Post-dose time points are in reference to the dose start time.
^{D}Events may occur ±2 days of indicated day beyond day 14 to accommodate scheduling
*Methylprednisolone continued weekly beyond Day 1 guided by exam and clinical pathology findings
**diphenhydramine 5 mg/kg intramuscularly three times daily from Day −1 through Day 1

TABLE 29
Groups 5 and 9 Study Schedule (n = 3)

| | | Study Interval^{D} | | | | | | | | | | | | | | |
|---|---|---|---|---|---|---|---|---|---|---|---|---|---|---|---|---|
| | | | | Day 0 | | | | | | | | | | | | |
| | | Base- | Day | 0 | 1 | 4 | Day | Day | Day | Day | Day | Day | Day | Day | Day | Day |
| Event | # | line | −1 | hour | hours^{T} | hours^{T} | 1 | 3 | 7 | 14 | 21 | 30 | 45 | 60 | 75 | 85 |
| Dosing | 3 | — | — | X | — | — | — | — | — | — | — | — | — | — | — | — |
| Methylprednisolone* | 3 | — | X | — | — | — | X | — | X | — | — | — | — | — | — | — |
| Diphenhydramine** | 3 | — | X | X | — | — | X | — | — | — | — | — | — | — | — | — |
| Body weights | 3 | X | — | X | — | — | X | — | X | — | — | X | — | X | — | X |
| Physiology-ECG, RR, O₂ | 3 | X | — | — | — | — | X | — | X | — | — | X | — | X | — | X |
| Physical exam | 3 | X | — | — | — | — | X | — | X | — | — | X | — | — | — | X |
| Clinical chemistry | 3 | X | — | — | — | — | X | X | X | — | — | X | — | X | — | X |
| Complete blood count | 3 | X | — | — | — | — | X | X | X | — | — | X | — | X | — | X |
| Coagulation profile | 3 | X | — | — | — | — | X | X | X | — | — | X | — | X | — | X |
| Biomarker plasma | 3 | X | — | — | X | X | X | X | X | — | — | X | — | X | — | X |
| Serum | 3 | X | — | — | X | X | X | X | X | — | — | X | — | X | — | X |
| Semen*** | 1 | — | — | — | — | — | — | — | X | — | — | — | — | — | — | — |
| Necropsy and tissues | 3 | — | — | — | — | — | — | — | — | — | — | — | — | — | — | X |

X = event for animals in each dosing group with respect to respective Day 0 dosing day
^{T}Post-dose time points are in reference to the dose start time.
^{D}Events may occur ±2 days of indicated day beyond day 14 to accommodate scheduling
*Methylprednisolone continued weekly beyond Day 1 guided by exam and clinical pathology findings
**diphenhydramine 5 mg/kg intramuscularly three times daily from Day −1 through Day 1
***Day 7 semen collection from the single male in Group 9

TABLE 30
Group Study Schedule (n = 1)

| | | Study Interval^{D} | | | | | | | | | | | | | | |
|---|---|---|---|---|---|---|---|---|---|---|---|---|---|---|---|---|
| | | | | Day 0 | | | | | | | | | | | | |
| | | Base- | Day | 0 | 1 | 4 | Day | Day | Day | Day | Day | Day | Day | Day | Day | Day |
| Event | # | line | −1 | hour | hours^{T} | hours^{T} | 1 | 3 | 7 | 14 | 21 | 30 | 45 | 60 | 75 | 85 |
| Dosing | 1 | — | — | X | — | — | — | — | — | — | — | — | — | — | — | — |
| Methylprednisolone* | 1 | — | X | — | — | — | X | — | X | — | — | — | — | — | — | — |
| Diphenhydramine** | 1 | — | X | X | — | — | X | — | — | — | — | — | — | — | — | — |
| Body weights | 1 | X | — | X | — | — | X | — | X | — | — | X | — | X | — | X |
| Physiology-ECG, RR, O₂ | 1 | X | — | — | — | — | X | — | X | — | — | X | — | X | — | X |
| Physical exam | 1 | X | — | — | — | — | X | — | X | — | — | X | — | — | — | X |
| Clinical chemistry | 1 | X | — | — | — | — | X | X | X | — | — | X | — | X | — | X |
| Complete blood count | 1 | X | — | — | — | — | X | X | X | — | — | X | — | X | — | X |
| Coagulation profile | 1 | X | — | — | — | — | X | X | X | — | — | X | — | X | — | X |
| Biomarker plasma | 1 | X | — | — | X | X | X | X | X | — | — | X | — | X | — | X |
| Serum | 1 | X | — | — | X | X | X | X | X | — | — | X | — | X | — | X |
| Semen*** | 0 | — | — | — | — | — | — | — | — | — | — | — | — | — | — | — |
| Necropsy and tissues | 1 | — | — | — | — | — | — | — | — | — | — | — | — | — | — | X |

X = event for animals in each dosing group with respect to respective Day 0 dosing day
^{T}Post-dose time points are in reference to the dose start time.
^{D}Events may occur ±2 days of indicated day beyond day 14 to accommodate scheduling
*Methylprednisolone continued weekly beyond Day 1 guided by exam and clinical pathology findings
**diphenhydramine 5 mg/kg intramuscularly three times daily from Day −1 through Day 1

TABLE 31
Groups 9 and 10 Study Schedule (n = 2)

| | | Study Interval^{D} | | | | | | | | | | | | | | |
|---|---|---|---|---|---|---|---|---|---|---|---|---|---|---|---|---|
| | | | | Day 0 | | | | | | | | | | | | |
| | | Base- | Day | 0 | 1 | 4 | Day | Day | Day | Day | Day | Day | Day | Day | Day | Day |
| Event | # | line | −1 | hour | hours^{T} | hours^{T} | 1 | 3 | 7 | 14 | 21 | 30 | 45 | 50 | 75 | 85 |
| Dosing | 2 | — | — | X | — | — | — | — | — | — | — | — | — | — | — | — |
| Methylprednisolone* | 2 | — | X | — | — | — | X | — | X | — | — | — | — | — | — | — |
| Diphenhydramine** | 2 | — | X | X | — | — | X | — | — | — | — | — | — | — | — | — |
| Body weights | 2 | X | — | X | — | — | X | — | X | — | — | X | — | X | — | — |
| Physiology-ECG, RR, O2 | 2 | X | — | — | — | — | X | — | X | — | — | X | — | X | — | — |
| Physical exam | 2 | X | — | — | — | — | X | — | X | — | — | X | — | X | — | — |
| Clinical chemistry | 2 | X | — | — | — | — | X | X | X | — | — | X | — | X | — | — |
| Complete blood count | 2 | X | — | — | — | — | X | X | X | — | — | X | — | X | — | — |
| Coagulation profile | 2 | X | — | — | — | — | X | X | X | — | — | X | — | X | — | — |
| Biomarker plasma | 2 | X | — | — | X | X | X | X | X | — | — | X | — | X | — | — |
| Serum | 2 | X | — | — | X | X | X | X | X | — | — | X | — | X | — | — |
| Semen*** | 1 | — | — | — | — | — | — | — | X | — | — | — | — | — | — | — |
| Necropsy and tissues | 2 | — | — | — | — | — | — | — | — | — | — | — | — | X | — | — |

X = event for animals in each dosing group with respect to respective Day 0 dosing day
^{T}Post-dose time points are in reference to the dose start time.
^{D}Events may occur ±2 days of indicated day beyond day 14 to accommodate scheduling
*Methylprednisolone continued weekly beyond Day 1 guided by exam and clinical pathology findings
**diphenhydramine 5 mg/kg intramuscularly three times daily from Day −1 through Day 1
***Day 7 semen collection from the single male in Group 10

TABLE 32
Group 11-13 Study Schedule (n = 5)

| | | Study Interval^{D} | | | | | | | | |
|---|---|---|---|---|---|---|---|---|---|---|
| | | | | | | 4.5 | Day | | | |
| | | | | | 1.5 | hours^{T} | 1 | | | |
| | | | | | hour^{T} | 270+/ | 1440+/ | | | |
| | | Base- | Day | 0 | 90+/−5 | −10 | 15 | Day | Day | Day |
| Event | # | line | -1 | hour | minutes | minutes | minutes | 3 | 7 | 21 |
| Dosing | 5 | — | — | X | — | — | — | — | — | — |
| Methylprednisolone* | 5 | — | X | — | — | — | X | — | X | — |
| Diphenhydramine** | 5 | — | X | X | — | — | X | — | — | — |
| Body weights | 5 | X | — | X | — | — | X | — | X | X |
| Physiology-ECG, RR, O2 | 5 | X | — | — | — | — | X | — | X | X |
| Physical exam | 5 | X | — | — | — | — | X | — | X | X |
| Clinical chemistry | 5 | X | — | — | — | — | X | X | X | X |
| Complete blood count | 5 | X | — | — | — | — | X | X | X | X |
| Coagulation profile | 5 | X | — | — | — | — | X | X | X | X |
| Biomarker plasma | 5 | X | — | — | X | X | X | X | X | X |
| Serum | 5 | X | — | — | X | X | X | X | X | X |
| Necropsy and tissues | 5 | — | — | — | — | — | — | — | — | X |

X = event for animals in each dosing group with respect to respective Day 0 dosing day
^{T}Post-dose time points are in reference to the dose start time.
^{D}Events may occur ±4 days of indicated day beyond day 14 to accommodate scheduling
*Methylprednisolone continued weekly beyond Day 1 guided by exam and clinical pathology findings
**diphenhydramine 5 mg/kg intramuscularly three times daily from Day −1 through Day 1

Clinical Observations: Cage side evaluations of general well-being assessing animal health, behavior, and food consumption were performed twice a day, beginning one week prior to dosing, and extending to the study terminus.

Body Weights: Body weights were collected at designated time points (Tables 28-32).

Physical Exam and Physiology: Detailed physical exam and physiology assessments will be performed at designated time points (Tables 28-32), including:

Integument exam: The integrity of the skin, hair, and palpable anatomy was assessed.

Respiratory rate: The number of breaths per minute was measured during a 20 second interval by direct visualization during pulmonary auscultation.

Heart rate: The number of beats per minute during a 20 second interval was measured during cardiac auscultation; detected arrhythmias and murmurs were documented.

Electrocardiography (ECG) was performed using arm, leg, and precordial ECG leads (5 leads) and recorded at a speed of 25 mm/sec with a sensitivity of 10 mm/mV. Two representative traces ~20 seconds in duration were obtained.

Oxygen saturation was measured using a pulse oximeter.

Body temperature was measured using a digital rectal thermometer.

Non-invasive blood pressure was measured using a high-definition oscillometry (HDO) blood pressure monitor with the cuff placed around the base of the tail, recording, and averaging two measures.

Clinical Pathology: At designated time points (Tables 28-32) 3.5 mL blood was collected for clinical pathology including:

Complete blood count (CBC): 0.5 mL blood was transferred to K2EDTA lavender top vacutainers, gently inverted several times until CBC with differential analysis on an Abaxis VetScan HM5 hematology system was completed.

Clinical chemistry: 1 mL blood was collected and transferred to green top vacutainers (containing lithium heparin) inverted 3-5 times and centrifuged at 3000 rpm for 10 minutes at 4° C. to attain 0.5 ml of plasma for clinical chemistry analysis on an Abaxis VetScan VS2 clinical chemistry system.

Coagulation profile: 1.8 mL blood was collected and transferred to citrate anticoagulated blue topped vacutainers inverted 3-5 times until evaluation of prothrombin time (PT), activated partial thromboplastin time (aPTT) and fibrinogen on an Abaxis VetScan VSPro coagulation analysis system was completed.

D-Dimer profiles: One blood sample (1.0 mL) was transferred to a citrate anticoagulated blue topped tube, inverted 3×, centrifuged at 3000 rpm for 10 minutes at 4° C., and plasma (~0.5 mL) was transferred to a labeled cryotube, stored, and shipped below −70° C. to Antech Diagnostics GLP for D-Dimer analysis.

C-reactive protein: One blood sample (1.0 mL) was collected and retained in red topped tubes for 30-60 minutes at room temperature followed by centrifugation at 3000 rpm for 10 minutes at 4° C. A serum aliquot (~0.5 mL or full volume) was transferred to a pre-labeled cryotube, stored, and shipped below −70° C. to Antech Diagnostics GLP for C-Reactive protein analysis.

Biomarker Plasma: At designated time points (Tables 28-32) blood (1 mL) was collected in K3EDTA anti-coagulated lavender topped tubes, inverted 3×, and maintained on ice (<30 minutes) until preparation of plasma by centrifugation at 3000 rpm for 10 minutes at 4° C. was performed. Plasma (~0.5 mL) was aliquoted to pre-labeled cryotubes, stored, and shipped below −70° C. to an off-site laboratory for multiplex cytokine analysis.

Serum: At designated time points (Tables 28-32) blood (1.5 mL) was collected for serum preparation by incubation in centrifuge tubes (without clot activators) for 1 hour at room temperature to allow clotting followed by centrifugation at 3000 rpm for 10 minutes at 4° C. Serum (~0.25-0.75 mL aliquots×2) was transferred to pre-labeled cryotubes, stored, and shipped below −70° C. to an off-site laboratory for biomarker analysis.

Semen Sample Collection: At designated time points (Tables 28-32) following overnight fasting, monkeys were sedated by intramuscular administration of ketamine/xylazine (8 mg/kg and 1.6 mg/kg, respectively). Using a sterile gauze, the penis was cleaned with 0.9% sodium chloride. The penis was placed into a pre-tarred 15 ml lobind falcon tube before the procedure began. An electroejaculation probe was inserted into the rectum in proximity to the prostate and activated by slowly increasing the voltage (automatic setting) until the monkey responded to the stimulus with leg extensions. The penis remained in the 15 ml lobind falcon tube during this time, as time of ejaculate varies across each monkey (if a sample was unable to be obtained following maximum stimulation, the probe was removed, and the monkey was allowed to remain unstimulated for ~2 mins before another attempt at collection was made. If no sample was obtained after 3 attempts, the procedure was stopped, and no sample was collected at that time point. The collected semen sample was transferred to a pre-labeled cryotube, stored, and shipped below −70° C. to an off-site laboratory for biomarker analysis.

Unexpected Moribundity/Mortality: Mortality and morbidity observations were conducted twice each day of the study. Any decision regarding premature sacrifice due to animal well-being is made based on the recommendations of a facility veterinarian. If moribund, clinical pathology is conducted, and other evaluations are defined in consultation with facility veterinarians. If an animal is discovered deceased, a full diagnostic necropsy is performed, collecting heart, lung, liver, kidney, spleen, brain, and observed lesions to determine the cause of death.

Termination: Animals were euthanized at the study terminus with ketamine (8 mg/kg IM) and xylazine (1.6 mg/kg IM) followed by sodium pentobarbital (100 mg/kg) administered intravascularly over a period of ~10 seconds to effect. After loss of corneal reflex, and prior to sample collection, animals were exsanguinated by incising the caudal vena cava followed by immediate perforation of the diaphragm relieving negative intrathoracic pressure.

Necropsy: Post-mortem veterinary examination of organs including external features of the carcass, external body orifices, abdominal, thoracic, and cranial cavities, organs, and tissues were performed to identify and document any gross abnormality or pathology.

Tissue Collection: Tissues collected from Necropsy were collected for formalin fixation and paraffin embedding (FFPE) followed by staining with hematoxylin and eosin (H&E). Tissues were collected in a IL screw top containers filled ⅔ with 10% neutral buffered formalin (NBF) and were labeled to indicate Animal #Study #Date. Parallel flash frozen tissues were collected for bioanalysis.

Ocular tissues: Right globes (OD) with adnexa (eye with bulbar conjunctivae, upper and lower eyelids with palpebral conjunctivae, harderian gland, lacrimal gland, optic nerve, extraocular muscles) were gently dissected without tension or traction on the optic nerve, followed by immersion fixation in Davidson's solution with >20:1 solution to tissue ratio at room temperature for 18-24 hours then transfer into phosphate buffered saline (PBS) with 0.05% sodium azide. Globes were stored and shipped at 3-8° C. in a Credo Cube container. Enucleated left globes (OS) were subdissected into cornea, iris/ciliary body, cornea, vitreous, retina, retinal pigment epithelium/choroid, sclera and optic nerve and transferred to a pre-labeled cryotube, flash frozen, stored, and shipped below −70° C. to an off-site laboratory.

Systemic tissues: Tissues were collected, with sample thickness not exceeding 5-10 mm, and immersion fixed in 10% NBF with >10:1 solution to tissue ratio for 24 hours at room temperature. Then NBF was decanted and replaced with fresh 10% NBF and tissues fixed an addition 24-48 hours. NBF was then decanted and replaced with 70% ethanol, and tissues allowed to remain immersed at room temperature until preparation for shipping. At shipping, all fixed systemic tissues will be thoroughly rinsed in phosphate buffered saline (PBS) with 0.05% sodium azide and transferred from their IL screw top fixation vessels to heat seal for shipment in a Credo Cube container conditioned to maintain room temperature. Parallel samples will be transferred to a pre-labeled cryotube, flash frozen, stored and shipped below −70° C. to an off-site laboratory.

CNS tissues: The right hemisphere of the brain and spinal cord segments from the cervical, thoracic and lumber regions were immersion fixed intact in 10% NBF, maintaining solution to tissue ratio of >10:1 for 24 hours at room temperature. NBF was decanted and replaced with fresh 10% NBF and the brain was allowed to fix an additional 24-48 hours. Then NBF was replaced with fresh 10% NBF and shipped in a Credo Cube container conditioned to maintain room temperature, and retained in 10% NBF for a minimum of 30 days prior to trimming, processing, and embedding. In the left hemisphere, the following neuroanatomic areas were sampled from 4 mm coronal sections and transferred to pre-labeled cryotubes, flash frozen, stored, and shipped below −70° C. to an off-site laboratory: caudate/putamen, cerebral cortex (parietal, temporal), corpus callosum, anterior commissure, hypothalamus, amygdala, thalamus, hippocampus, optic tract, trigeminal nuclei and medulla oblongata.

Histopathology: 10% NBF-fixed tissues were processed at Virscio's Pathology and Tissue Pharmacodynamics laboratory for histopathology. Specimens were trimmed, processed, embedded in paraffin, sectioned in stepped sections, and H&E stained for whole slide imaging on a 3DHisTech whole slide imager and evaluated by a board-certified veterinary pathologist.

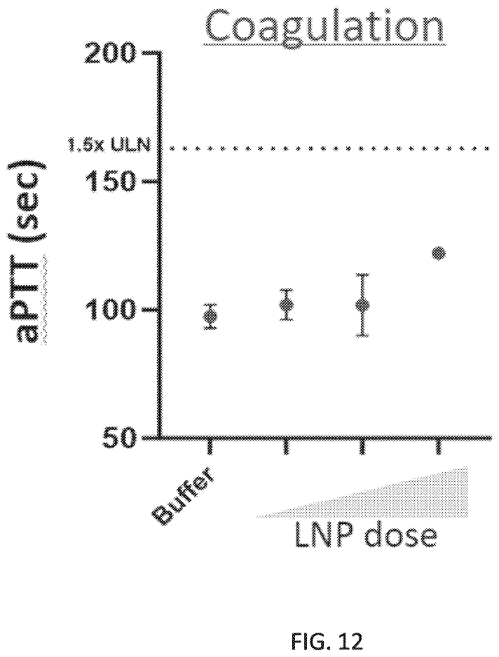

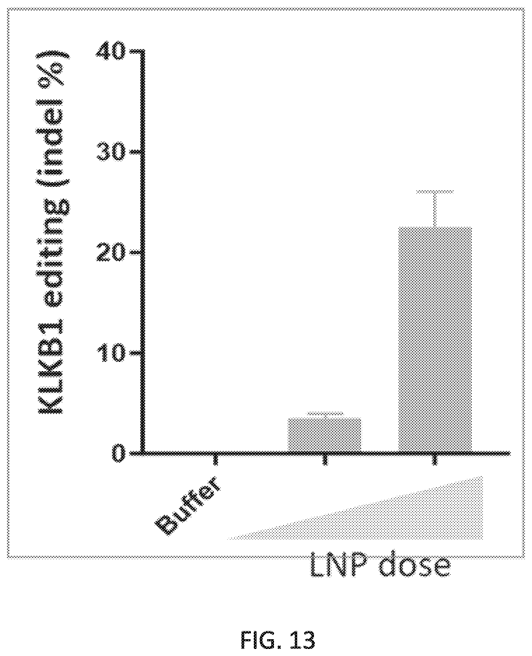

Interim Results: No dose-limiting toxicity was observed up to maximum dose tested. No changes in clotting nor evidence of coagulopathy (aPTT) were observed (FIG. 12). The minimal elevation of hepatotoxicity markers was rapidly resolved. Without wishing to be bound by theory, the interim efficacy data indicated successful dose-responsive liver editing (FIG. 13).

1. A composition comprising:
(a) a first guide RNA (gRNA) comprising a first targeting sequence set forth in SEQ ID Nos: 1-17, 49, 50, or 55;
(b) a second gRNA comprising a second targeting sequence set forth in SEQ ID NOs: 18-34, 51, 52, or 56;
(c) a first polynucleotide encoding a first fusion protein, wherein the first fusion protein comprises a first inactivated Cas9 domain, or nuclease domain thereof, and a first Clo051 domain, or nuclease domain thereof; and
(d) a second polynucleotide encoding a second fusion protein, wherein the second fusion protein comprises a second inactivated Cas9 domain, or nuclease domain thereof, and a second Clo051 domain, or nuclease domain thereof.

2. The composition of claim 1, wherein the C-terminus of the first fusion protein further comprises a linker comprising the sequence set forth in SEQ ID NO: 57 between the first inactivated Cas9 domain, or nuclease domain thereof, and the first Clo051 domain, or nuclease domain thereof, and/or the second fusion protein further comprises a linker comprising the sequence set forth in SEQ ID NO: 57 between the second inactivated Cas9 domain, or nuclease domain thereof, and the second Clo051 domain, or nuclease domain thereof.

3. The composition of claim 1 or claim 2, wherein the first fusion protein comprises the amino acid sequence set forth in SEQ ID NO: 35 or SEQ ID NO: 38, and/or the second fusion protein comprises the amino acid sequence set forth in SEQ ID NO: 35 or SEQ ID NO: 38.

4. The composition of claim 1 or claim 2, wherein the first polynucleotide is an mRNA comprising the sequence set forth in SEQ ID NO: 36 or SEQ ID NO: 39, and/or the second polynucleotide is an mRNA comprising the sequence set forth in SEQ ID NO: 36 or SEQ ID NO: 39.

5. The composition of claim 4, wherein the mRNA comprises a 5′-cap.

6. The composition of any one of claims 1-5, wherein the first inactivated Cas9 domain is derived from a Streptococcus pyogenes Cas9 polypeptide, and/or the second inactivated Cas9 domain is derived from a Streptococcus pyogenes Cas9 polypeptide.

7. The composition of any one of claims 1-6, wherein the first gRNA comprises a spacer and a scaffold sequence isolated from Streptococcus pyogenes, and/or the second gRNA comprises a spacer and a scaffold sequence isolated from Streptococcus pyogenes.

8. The composition of claim 7, wherein the scaffold sequence comprises the sequence set forth in SEQ ID NO: 48.

9. The composition of any one of claims 1-8, wherein the first gRNA comprises one or more chemical modifications of a ribonucleotide, a ribonucleotide base, or a phosphodiester bond, and/or the second gRNA comprises one or more chemical modifications of a ribonucleotide, a ribonucleotide base, or a phosphodiester bond.

10. The composition of claim 9, wherein the chemical modification comprises a phosphorothioate bond.

11. The composition of claim 9 or claim 10, wherein the 5′- and/or 3′-terminus of the first gRNA comprises at least two consecutive phosphorothioate bonds, and/or the 5′- and/or 3′-terminus of the second gRNA comprises at least two consecutive phosphorothioate bonds.

12. The composition of claim 9, wherein the 5′- and/or 3′-terminus of the first gRNA comprises at least one 2′ O-Me chemical modification, and/or the 5′- and/or 3′-terminus of the second gRNA comprises at least one 2′ O-Me chemical modification.

13. The composition of any one of claims 1-12, wherein
(a) the first gRNA comprises the sequence set forth in SEQ ID NO: 1 and the second gRNA comprises the sequence set forth in SEQ ID NO: 18;
(b) the first gRNA comprises the sequence set forth in SEQ ID NO: 2 and the second gRNA comprises the sequence set forth in SEQ ID NO: 19
(c) the first gRNA comprises the sequence set forth in SEQ ID NO: 3 and the second gRNA comprises the sequence set forth in SEQ ID NO: 20,
(d) the first gRNA comprises the sequence set forth in SEQ ID NO: 4 and the second gRNA comprises the sequence set forth in SEQ ID NO: 21,
(e) the first gRNA comprises the sequence set forth in SEQ ID NO: 5 and the second gRNA comprises the sequence set forth in SEQ ID NO: 22,
(f) the first gRNA comprises the sequence set forth in SEQ ID NO: 6 and the second gRNA comprises the sequence set forth in SEQ ID NO: 23,
(g) the first gRNA comprises the sequence set forth in SEQ ID NO: 7 and the second gRNA comprises the sequence set forth in SEQ ID NO: 24,
(h) the first gRNA comprises the sequence set forth in SEQ ID NO: 8 and the second gRNA comprises the sequence set forth in SEQ ID NO: 25,
(j) the first gRNA comprises the sequence set forth in SEQ ID NO: 9 and the second gRNA comprises the sequence set forth in SEQ ID NO: 26,
(k) the first gRNA comprises the sequence set forth in SEQ ID NO: 10 and the second gRNA comprises the sequence set forth in SEQ ID NO: 27,
(l) the first gRNA comprises the sequence set forth in SEQ ID NO: 11 and the second gRNA comprises the sequence set forth in SEQ ID NO: 28,
(m) the first gRNA comprises the sequence set forth in SEQ ID NO: 12 and the second gRNA comprises the sequence set forth in SEQ ID NO: 29,
(n) the first gRNA comprises the sequence set forth in SEQ ID NO: 13 and the second gRNA comprises the sequence set forth in SEQ ID NO: 30,
(o) the first gRNA comprises the sequence set forth in SEQ ID NO: 14 and the second gRNA comprises the sequence set forth in SEQ ID NO: 31,
(p) the first gRNA comprises the sequence set forth in SEQ ID NO: 15 and the second gRNA comprises the sequence set forth in SEQ ID NO: 32,
(q) the first gRNA comprises the sequence set forth in SEQ ID NO: 16 and the second gRNA comprises the sequence set forth in SEQ ID NO: 33,
(r) the first gRNA comprises the sequence set forth in SEQ ID NO: 17 and the second gRNA comprises the sequence set forth in SEQ ID NO: 34,
(s) the first gRNA comprises the sequence set forth in SEQ ID NO: 49 and the second gRNA comprises the sequence set forth in SEQ ID NO: 51,
(t) the first gRNA comprises the sequence set forth in SEQ ID NO: 50 and the second gRNA comprises the sequence set forth in SEQ ID NO: 52, or
(u) the first gRNA comprises the sequence set forth in SEQ ID NO: 55 and the second gRNA comprises the sequence set forth in SEQ ID NO: 56.

14. The composition of any one of claims 1-12, wherein the first and second fusion proteins comprise the amino acid sequence set forth in SEQ ID NO: 35, and wherein
(a) the first gRNA comprises the sequence set forth in SEQ ID NO: 1 and the second gRNA comprises the sequence set forth in SEQ ID NO: 18;
(b) the first gRNA comprises the sequence set forth in SEQ ID NO: 2 and the second gRNA comprises the sequence set forth in SEQ ID NO: 19
(c) the first gRNA comprises the sequence set forth in SEQ ID NO: 3 and the second gRNA comprises the sequence set forth in SEQ ID NO: 20,
(d) the first gRNA comprises the sequence set forth in SEQ ID NO: 4 and the second gRNA comprises the sequence set forth in SEQ ID NO: 21,
(e) the first gRNA comprises the sequence set forth in SEQ ID NO: 5 and the second gRNA comprises the sequence set forth in SEQ ID NO: 22,
(f) the first gRNA comprises the sequence set forth in SEQ ID NO: 6 and the second gRNA comprises the sequence set forth in SEQ ID NO: 23,
(g) the first gRNA comprises the sequence set forth in SEQ ID NO: 7 and the second gRNA comprises the sequence set forth in SEQ ID NO: 24,
(h) the first gRNA comprises the sequence set forth in SEQ ID NO: 8 and the second gRNA comprises the sequence set forth in SEQ ID NO: 25,
(j) the first gRNA comprises the sequence set forth in SEQ ID NO: 9 and the second gRNA comprises the sequence set forth in SEQ ID NO: 26,
(k) the first gRNA comprises the sequence set forth in SEQ ID NO: 10 and the second gRNA comprises the sequence set forth in SEQ ID NO: 27,
(l) the first gRNA comprises the sequence set forth in SEQ ID NO: 11 and the second gRNA comprises the sequence set forth in SEQ ID NO: 28,
(m) the first gRNA comprises the sequence set forth in SEQ ID NO: 12 and the second gRNA comprises the sequence set forth in SEQ ID NO: 29,
(n) the first gRNA comprises the sequence set forth in SEQ ID NO: 13 and the second gRNA comprises the sequence set forth in SEQ ID NO: 30,
(o) the first gRNA comprises the sequence set forth in SEQ ID NO: 14 and the second gRNA comprises the sequence set forth in SEQ ID NO: 31,
(p) the first gRNA comprises the sequence set forth in SEQ ID NO: 15 and the second gRNA comprises the sequence set forth in SEQ ID NO: 32,
(q) the first gRNA comprises the sequence set forth in SEQ ID NO: 16 and the second gRNA comprises the sequence set forth in SEQ ID NO: 33,
(r) the first gRNA comprises the sequence set forth in SEQ ID NO: 17 and the second gRNA comprises the sequence set forth in SEQ ID NO: 34,
(s) the first gRNA comprises the sequence set forth in SEQ ID NO: 49 and the second gRNA comprises the sequence set forth in SEQ ID NO: 51,
(t) the first gRNA comprises the sequence set forth in SEQ ID NO: 50 and the second gRNA comprises the sequence set forth in SEQ ID NO: 52, or
(u) the first gRNA comprises the sequence set forth in SEQ ID NO: 55 and the second gRNA comprises the sequence set forth in SEQ ID NO: 56.

15. The composition of any one of claims 1-12, wherein the first and second fusion proteins comprise the amino acid sequence set forth in SEQ ID NO: 38, and wherein
(a) the first gRNA comprises the sequence set forth in SEQ ID NO: 1 and the second gRNA comprises the sequence set forth in SEQ ID NO: 18;
(b) the first gRNA comprises the sequence set forth in SEQ ID NO: 2 and the second gRNA comprises the sequence set forth in SEQ ID NO: 19
(c) the first gRNA comprises the sequence set forth in SEQ ID NO: 3 and the second gRNA comprises the sequence set forth in SEQ ID NO: 20,
(d) the first gRNA comprises the sequence set forth in SEQ ID NO: 4 and the second gRNA comprises the sequence set forth in SEQ ID NO: 21,
(e) the first gRNA comprises the sequence set forth in SEQ ID NO: 5 and the second gRNA comprises the sequence set forth in SEQ ID NO: 22,
(f) the first gRNA comprises the sequence set forth in SEQ ID NO: 6 and the second gRNA comprises the sequence set forth in SEQ ID NO: 23,
(g) the first gRNA comprises the sequence set forth in SEQ ID NO: 7 and the second gRNA comprises the sequence set forth in SEQ ID NO: 24,
(h) the first gRNA comprises the sequence set forth in SEQ ID NO: 8 and the second gRNA comprises the sequence set forth in SEQ ID NO: 25,
(j) the first gRNA comprises the sequence set forth in SEQ ID NO: 9 and the second gRNA comprises the sequence set forth in SEQ ID NO: 26,
(k) the first gRNA comprises the sequence set forth in SEQ ID NO: 10 and the second gRNA comprises the sequence set forth in SEQ ID NO: 27,
(l) the first gRNA comprises the sequence set forth in SEQ ID NO: 11 and the second gRNA comprises the sequence set forth in SEQ ID NO: 28,
(m) the first gRNA comprises the sequence set forth in SEQ ID NO: 12 and the second gRNA comprises the sequence set forth in SEQ ID NO: 29,
(n) the first gRNA comprises the sequence set forth in SEQ ID NO: 13 and the second gRNA comprises the sequence set forth in SEQ ID NO: 30,
(o) the first gRNA comprises the sequence set forth in SEQ ID NO: 14 and the second gRNA comprises the sequence set forth in SEQ ID NO: 31,
(p) the first gRNA comprises the sequence set forth in SEQ ID NO: 15 and the second gRNA comprises the sequence set forth in SEQ ID NO: 32,
(q) the first gRNA comprises the sequence set forth in SEQ ID NO: 16 and the second gRNA comprises the sequence set forth in SEQ ID NO: 33,
(r) the first gRNA comprises the sequence set forth in SEQ ID NO: 17 and the second gRNA comprises the sequence set forth in SEQ ID NO: 34,
(s) the first gRNA comprises the sequence set forth in SEQ ID NO: 49 and the second gRNA comprises the sequence set forth in SEQ ID NO: 51,
(t) the first gRNA comprises the sequence set forth in SEQ ID NO: 50 and the second gRNA comprises the sequence set forth in SEQ ID NO: 52, or
(u) the first gRNA comprises the sequence set forth in SEQ ID NO: 55 and the second gRNA comprises the sequence set forth in SEQ ID NO: 56.

16. The composition of any one of claims 1-15, wherein the composition is encapsulated in at least one Lipid Nanoparticle (LNP).

17. The composition of claim 16, wherein the at least one LNP comprises:
about 54% SS-OP by moles, about 35% cholesterol by moles, about 5% DOPC by moles, about 5% DSPC by moles, and about 1% DMG-PEG2000 by moles; or
about 54% SS-OP by moles, about 35% cholesterol by moles, about 10% DOPE by moles, and about 1% DMG-PEG2000 by moles; and
wherein the ratio of lipid to RNA in the at least one LNP is about 100:1 (w/w) and the total lipid is about 25 nM.

18. The composition of claim 16, wherein the at least one LNP comprises:
about 50% COMPOUND NO. 1 by moles, about 10% DOPC by moles, about 38.5% cholesterol by moles, and about 1.5% DMG-PEG2000 by moles; or
about 40% COMPOUND NO. 1 by moles, about 10% DOPC by moles, about 48.5% cholesterol by moles, and about 1.5% DMG-PEG2000 by moles;
wherein the ratio of lipid to RNA in the at least one LNP is about 80:1 (w/w), about 60:1 (w/w), or about 50:1 (w/w) and
wherein COMPOUND NO. 1 comprises the following structure:

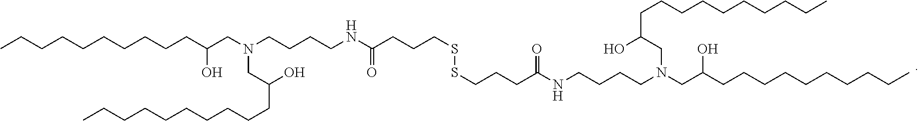 [SMILES: CCCCCCCCCCC(O)CN(CCCCNC(=O)CCCSSCCCC(=O)NCCCCN(CC(O)CCCCCCCCCC)CC(O)CCCCCCCCCC)CC(O)CCCCCCCCCC]

19. The composition of claim 16, wherein the at last one LNP comprises:
about 50% COMPOUND NO. 37 by moles, about 10% DSPC by moles, about 38.5% cholesterol by moles, and about 1.5% DMG-PEG2000 by moles;
about 50% COMPOUND NO. 37 by moles, about 10% DSPC by moles, about 38% cholesterol by moles, and about 2% DMG-PEG2000 by moles;
about 50% COMPOUND NO. 37 by moles, about 5% DSPC by moles, about 42% cholesterol by moles, and about 3% DMG-PEG2000 by moles; or
about 50% COMPOUND NO. 37 by moles, about 10% DOPC by moles, about 38% cholesterol by moles, and about 2% DMG-PEG2000 by moles,
wherein the ratio of lipid to RNA in the at least one LNP is about 50:1 (w/w), and
wherein COMPOUND NO. 37 comprises the following structure:

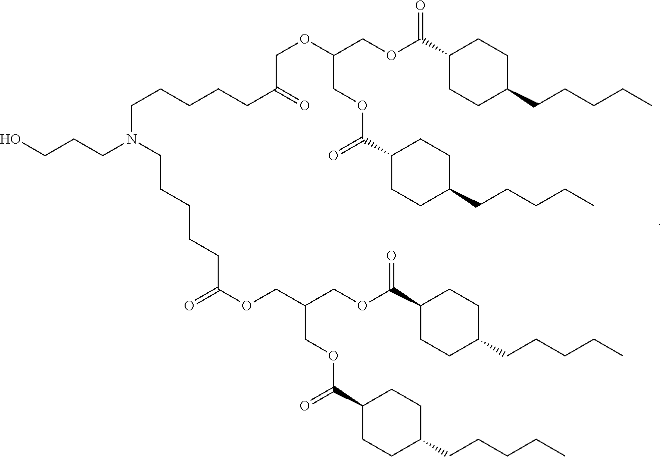 [SMILES: CCCCC[C@H]1CC[C@H](C(=O)OCC(COC(=O)CCCCCN(CCCO)CCCCCC(=O)COC(COC(=O)[C@H]2CC[C@H](CCCCC)CC2)COC(=O)[C@H]2CC[C@H](CCCCC)CC2)COC(=O)[C@H]2CC[C@H](CCCCC)CC2)CC1]

20. A method of modifying the genomes of a population of cells, comprising contacting the population of cells with the composition of any one of claims 1-19,
wherein the first fusion protein and second fusion protein are expressed by each cell of the population,
wherein the first gRNA is bound to the first fusion protein and the second gRNA is bound to the second fusion protein,
wherein the first gRNA specifically binds to a first strand of a first double-stranded DNA target sequence in each cell of the population, and
wherein the second gRNA specifically binds to a second strand of a second double-stranded DNA target sequence in each cell of the population.

21. The method of claim 20, wherein the first fusion protein and the second fusion protein introduces a modification into the genome of one or more cells in the population.

22. The method of claim 21, wherein the modification is an insertion or deletion (indel) between the first double-stranded DNA target sequence and the second double-stranded DNA target sequence.

23. The method of claim 22, wherein the indel causes the inactivation of a KLKB1 gene.

24. A population of cells modified according to the method of any one of claims 20-23.

25. The population of cells of claim 24, wherein the population of cells has a reduced level of KLKB1 protein expression relative to an unmodified population of cells.

26. The population of cells of claim 24 and claim 25, wherein the cells have at least a 50% reduction of KLKB1 protein expression relative to an unmodified population of cells.

27. The population of cells of any one of claims 24-26, wherein at least 20% of the cells in the population of cells include an indel at the KLKB1 locus.

28. A method of treating a Hereditary Angioedema in a subject in need thereof, comprising administering to the subject a therapeutically effective amount of the composition of any one of claims 1-19 or the population of cells of claim 24.

29. The method of claim 28, wherein the Hereditary Angioedema is Hereditary Angioedema Type 1 or Hereditary Angioedema Type 2.

30. Use of the composition of any one of claims 1-19 or the population of cells of claim 24 for the manufacture of a medicament for the treatment of Hereditary Angioedema.

31. The use of claim 30, wherein the Hereditary Angioedema is Hereditary Angioedema Type 1 or Hereditary Angioedema Type 2.


SEQUENCE LISTING (58 sequences)

SEQ ID NO: 1 - RNA, 20 nt, synthetic construct
  source 1..20: mol_type = other RNA
tgccattctaaaatttaccc

SEQ ID NO: 2 - RNA, 20 nt, synthetic construct
  source 1..20: mol_type = other RNA
acaagcacacgcattgttgg

SEQ ID NO: 3 - RNA, 20 nt, synthetic construct
  source 1..20: mol_type = other RNA
tatggaaaatcgagtcacaa

SEQ ID NO: 4 - RNA, 20 nt, synthetic construct
  source 1..20: mol_type = other RNA
ctgctttgatgggtaagtgt

SEQ ID NO: 5 - RNA, 20 nt, synthetic construct
  source 1..20: mol_type = other RNA
ttcttaagattatctatgga

SEQ ID NO: 6 - RNA, 20 nt, synthetic construct
  source 1..20: mol_type = other RNA
gtcacaaaggcgagtatgca

SEQ ID NO: 7 - RNA, 20 nt, synthetic construct
  source 1..20: mol_type = other RNA
tgccattctaaaatttaccc

SEQ ID NO: 8 - RNA, 20 nt, synthetic construct
  source 1..20: mol_type = other RNA
ctgtgtggagggtcactcat

SEQ ID NO: 9 - RNA, 20 nt, synthetic construct
  source 1..20: mol_type = other RNA
tatggaaaatcgagtcacaa

SEQ ID NO: 10 - RNA, 20 nt, synthetic construct
  source 1..20: mol_type = other RNA
tttgagattgtgtaacactg

SEQ ID NO: 11 - RNA, 20 nt, synthetic construct
  source 1..20: mol_type = other RNA
ggtgttttcttgaggagtag

SEQ ID NO: 12 - RNA, 20 nt, synthetic construct
  source 1..20: mol_type = other RNA
tcttgaggagtagaggaact

SEQ ID NO: 13 - RNA, 20 nt, synthetic construct
  source 1..20: mol_type = other RNA
cacctgcaggctcacctgcc

SEQ ID NO: 14 - RNA, 20 nt, synthetic construct
  source 1..20: mol_type = other RNA
acctgcaggctcacctgcca

SEQ ID NO: 15 - RNA, 20 nt, synthetic construct
  source 1..20: mol_type = other RNA
ctatgagtgaccctccacac

SEQ ID NO: 16 - RNA, 20 nt, synthetic construct
  source 1..20: mol_type = other RNA
ctatgagtgaccctccacac

SEQ ID NO: 17 - RNA, 20 nt, synthetic construct
  source 1..20: mol_type = other RNA
ctatgagtgaccctccacac

SEQ ID NO: 18 - RNA, 20 nt, synthetic construct
  source 1..20: mol_type = other RNA
aacacaaaaagatatcagat

SEQ ID NO: 19 - RNA, 20 nt, synthetic construct
  source 1..20: mol_type = other RNA
aaagagagcgttgacgctaa

SEQ ID NO: 20 - RNA, 20 nt, synthetic construct
  source 1..20: mol_type = other RNA
gttggggtgataggtgcaga

SEQ ID NO: 21 - RNA, 20 nt, synthetic construct
  source 1..20: mol_type = other RNA
ctatgagtgaccctccacac

SEQ ID NO: 22 - RNA, 20 nt, synthetic construct
  source 1..20: mol_type = other RNA
agaaagagtaagccttcaca

SEQ ID NO: 23 - RNA, 20 nt, synthetic construct
  source 1..20: mol_type = other RNA
ttgtatagaatgtaaagaag

SEQ ID NO: 24 - RNA, 20 nt, synthetic construct
  source 1..20: mol_type = other RNA
aaacacaaaaagatatcaga

SEQ ID NO: 25 - RNA, 20 nt, synthetic construct
  source 1..20: mol_type = other RNA
acctgcaggctcacctgcca

SEQ ID NO: 26 - RNA, 20 nt, synthetic construct
  source 1..20: mol_type = other RNA
gaagaggcagttggggtgat

SEQ ID NO: 27 - RNA, 20 nt, synthetic construct
  source 1..20: mol_type = other RNA
gtcccatacgcaatcctagt

SEQ ID NO: 28 - RNA, 20 nt, synthetic construct
  source 1..20: mol_type = other RNA
acctgcaaaagaactttacc

SEQ ID NO: 29 - RNA, 20 nt, synthetic construct
  source 1..20: mol_type = other RNA
acctgcaaaagaactttacc

SEQ ID NO: 30 - RNA, 20 nt, synthetic construct
  source 1..20: mol_type = other RNA
ctgtgtggagggtcactcat

SEQ ID NO: 31 - RNA, 20 nt, synthetic construct
  source 1..20: mol_type = other RNA
ctgtgtggagggtcactcat

SEQ ID NO: 32 - RNA, 20 nt, synthetic construct
  source 1..20: mol_type = other RNA
actgctgcccactgctttga

SEQ ID NO: 33 - RNA, 20 nt, synthetic construct
  source 1..20: mol_type = other RNA
ctgctgcccactgctttgat

SEQ ID NO: 34 - RNA, 20 nt, synthetic construct
  source 1..20: mol_type = other RNA
ctgctttgatgggtaagtgt

SEQ ID NO: 35 - protein, 1591 aa, synthetic construct
MAPKKKRKVEGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 36 - RNA, 4779 nt, synthetic construct
  source 1..4779: mol_type = other RNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa

SEQ ID NO: 37 - DNA, 4779 bp, synthetic construct
  source 1..4779: mol_type = other DNA
atggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataa

SEQ ID NO: 38 - protein, 1591 aa, synthetic construct
MAPKKKRKVEGIKSNISLLKDELRGQISHISHEYLSLIDLAFDPKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKYGGGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVSS

SEQ ID NO: 39 - RNA, 5124 nt, synthetic construct
  source 1..5124: mol_type = other RNA
acatttgcttctgacacaactgtgttcactagcaacctcaaacagacaccaagcttgccaccatggctcccaagaagaagcggaaggtcgagggcatcaagagcaacatcagcctgctgaaggacgagctgagaggccagatcagccacatctcccacgagtacctgagcctgatcgacctggccttcgaccccaagcagaaccggctgttcgagatgaaggtgctggaactgctggtcaacgagtacggcttcaagggcagacacctcggcggcagcagaaagcctgatggcatcgtgtacagcaccacactcgaggacaacttcggcatcatcgtggacaccaaggcctacagcgagggctacagcctgcctatctctcaggccgacgagatggaaagatacgtgcgcgagaacagcaaccgcgacgaggaagtgaaccccaacaagtggtgggagaacttcagcgaggaagtcaaaaagtactacttcgtgttcatcagcggcagctttaagggcaagttcgaggaacagctgcggcggctgtctatgaccacaggcgttaacggcagcgccgtgaacgtggtcaatctgctgctgggcgccgagaagattagaagcggcgagatgaccatcgaggaactggaacgggccatgttcaacaacagcgagttcatcctgaagtacggcggaggcggcagcgacaagaagtactctatcggactggccatcggcaccaactctgttggatgggccgtgatcaccgacgagtacaaggtgcccagcaagaaattcaaagtgctgggcaacaccgaccggcacagcatcaagaagaatctgatcggcgccctgctgttcgactctggcgaaacagccgaagccaccagactgaagagaaccgccagacggcggtacaccagaagaaagaaccggatctgctacctgcaagagatcttcagcaacgagatggccaaggtggacgacagcttcttccacagactggaagagtccttcctggtggaagaggacaagaagcacgagcggcaccccatcttcggaaatatcgtggacgaggtggcctaccacgagaagtaccccaccatctaccacctgagaaagaaactggtggacagcaccgacaaggccgacctgcgactgatctatctggccctggctcacatgatcaagttccggggccacttcctgatcgagggcgacctgaatcctgacaactccgacgtggacaagctgttcatccagctggtgcagacctacaatcagctgttcgaagagaatcccatcaacgcctctggcgtggacgccaaagccatcctgtctgccagactgagcaagagcagacggctggaaaacctgatcgctcagctgcccggcgagaagaagaatggcctgttcggcaacctgattgccctgtctctgggcctgacacctaacttcaagtccaacttcgatctggccgaggatgccaaactgcagctgtccaaggacacctacgacgacgacctggataacctgctggcccagatcggcgatcagtacgccgacttgtttctggccgccaagaacctgtctgacgccatcctgctgagcgacatcctgagagtgaacaccgagatcacaaaggcccctctgagcgcctctatgatcaagagatacgacgagcaccaccaggatctgaccctgctgaaagctctcgtcaggcagcagctgccagagaagtacaaagagattttcttcgaccagagcaagaacggctacgccggctacattgatggcggagccagccaagaggaattctacaagttcatcaagcccatcctcgagaagatggacggcacagaggaactgctcgtgaagctgaacagagaggacctgctgcggaagcagcggaccttcgacaatggctctatccctcaccagatccacctgggagagctgcacgccattctgcggagacaagaggacttttacccattcctgaaggacaaccgggaaaagattgagaagatcctgaccttcaggatcccctactacgtgggaccactggccagaggcaatagcagattcgcctggatgaccagaaagagcgaggaaaccatcacaccctggaacttcgaagaggtggtggacaagggcgccagcgctcagtccttcatcgagcggatgaccaatttcgacaagaatctgcccaacgagaaagtgctgcccaagcactccctgctgtacgagtacttcaccgtgtacaacgagctgaccaaagtgaaatacgtgaccgagggaatgagaaagcccgcctttctgtccggcgagcagaaaaaggccatcgtggatctgctgttcaagaccaaccggaaagtgaccgtgaagcagctgaaagaggactacttcaagaaaatcgagtgcttcgactccgtggaaatcagcggcgtggaagatcggttcaatgccagcctgggcacataccacgatctgctgaaaattatcaaggacaaggacttcctggacaacgaggaaaacgaggacatccttgaggacatcgtgctgaccctgacactgttcgaggacagagagatgatcgaggaaaggctgaaaacatacgcccacctgttcgacgacaaagtcatgaagcaactgaagcggcggcgctacacaggctggggcagactgtctagaaagctgatcaacggcatccgggacaagcagtccggcaagaccatcctggactttctgaagtccgacggcttcgccaacagaaacttcatgcagctgattcacgacgacagcctcaccttcaaagaggacattcagaaggcccaggtttccggccagggcgattctctgcacgagcacattgccaatctggccggctctcccgccattaagaagggcattctgcagacagtgaaagtggtggatgagctggtcaaagtgatggggagacacaagcccgagaacatcgtgatcgaaatggccagagagaaccagaccacacagaagggccagaagaactcccgcgagagaatgaagcggatcgaagagggaatcaaagagctggggagccagatcctgaaagaacaccccgtggaaaacacccagctgcagaacgagaagctgtacctgtactacctccagaacggccgggatatgtacgtggaccaagagctggacatcaaccgcctgagcgactacgatgtggacgctatcgtgccccagtcttttctgaaagatgactccatcgacaacaaggtgctgaccagaagcgataagaaccggggcaagagcgacaacgtgccctctgaagaggtcgtgaagaagatgaagaactactggcgacagctgctgaacgccaagctgattacccagcggaagttcgataacctgaccaaggccgagagaggcggcctgtctgaactggataaggccggcttcatcaagagacagctggtggaaacccggcagatcaccaaacacgtggcacagattctggactcccggatgaacaccaaatacgatgagaacgacaaactgatccgggaagtgaaagtcatcaccctgaagtccaagctggtgtccgatttccggaaggatttccagttctacaaagtgcgggaaatcaacaactaccatcacgcccacgacgcctacctgaatgccgttgttggaacagccctgatcaagaagtatcccaagctggaaagcgagttcgtgtacggcgactacaaggtgtacgacgtgcggaagatgatcgccaagagcgagcaagagattggaaaggctaccgccaaatacttcttctactccaacatcatgaactttttcaagacagagatcaccctcgccaacggcgagatcagaaagcggcctctgatcgagacaaacggcgaaaccggcgagattgtgtgggataagggcagagactttgccacagtgcggaaggtgctcagcatgccccaagtgaatatcgtgaaaaagaccgaggtgcagacaggcggcttcagcaaagagtccattctgcctaagcggaactccgacaagctgatcgcccggaagaaggactgggaccccaagaaatacggcggcttcgatagccctaccgtggcctattctgtgctggtggtggccaaagtggaaaagggaaagtccaagaagctcaagagcgtcaaagaactcctgggcatcaccatcatggaacggtccagcttcgagaagaaccctatcgactttctggaagccaagggctacaaagaagtcaagaaggacctgatcatcaagctccccaagtacagcctgttcgagctggaaaatggccggaagcggatgctggcttctgctggcgaactgcagaagggaaacgaactggccctgcctagcaaatatgtgaacttcctgtacctggccagccactatgagaagctgaagggcagccccgaggacaatgagcagaagcagcttttcgtcgagcagcacaagcactacctggacgagatcatcgagcagatctccgagttctccaagagagtgatcctggccgacgccaacctggacaaggttctgtccgcctacaacaagcaccgggataagcccatcagagagcaggccgagaatatcatccacctgtttaccctgaccaacctgggagcccctgccgccttcaagtacttcgacaccaccatcgaccggaagcgctacaccagcaccaaagaagtgctggacgccacactgatccaccagagcatcaccggcctgtacgagacacggatcgatctgtctcagcttggaggcgacggcagccctaagaagaagagaaaggtttccagctaataaggcggccgccctcgccccggacctgccctcccgccaggtgcacccacctgcaataaatgcagcgaagccgggagaattccctcgccccggacctgccctcccgccaggtgcacccacctgcaataaatgcagcgaagccgggagcggccgcggatccccgggtaccgaattcgatatctctatagtgtcacctaaatttaattaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 40 - protein, 1053 aa, synthetic construct
MKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 41 - protein, 1368 aa, synthetic construct
XDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 42 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 43 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 44 - protein, 1591 aa, synthetic construct; identical to SEQ ID NO: 35

SEQ ID NO: 45 - DNA, 4779 bp, synthetic construct; identical to SEQ ID NO: 37
  source 1..4779: mol_type = other DNA

SEQ ID NO: 46 - protein, 187 aa, synthetic construct
MVGSLNCIVAVSQNMGIGKNGDFPWPPLRNESRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 47 - DNA, 561 bp, synthetic construct
  source 1..561: mol_type = other DNA
atggtcgggtctctgaattgtatcgtcgccgtgagtcagaacatgggcattgggaagaatggcgatttcccatggccacctctgcgcaacgagtcccgatactttcagcggatgacaactacctcctctgtggaagggaaacagaatctggtcatcatgggaaagaaaacttggttcagcattccagagaagaaccggcccctgaaaggcagaatcaatctggtgctgtcccgagaactgaaggagccaccacagggagctcactttctgagccggtccctggacgatgcactgaagctgacagaacagcctgagctggccaacaaagtcgatatggtgtggatcgtcgggggaagttcagtgtataaggaggccatgaatcaccccggccatctgaaactgttcgtcacacggatcatgcaggactttgagagcgatactttctttcctgaaattgacctggagaagtacaaactgctgcccgaatatcctggcgtgctgtccgatgtccaggaagagaaaggcatcaaatacaagttcgaggtctatgagaagaatgac

SEQ ID NO: 48 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 49 - RNA, 20 nt, synthetic construct
  source 1..20: mol_type = other RNA
gagctgtcagcttcacctgc

SEQ ID NO: 50 - RNA, 20 nt, synthetic construct
  source 1..20: mol_type = other RNA
gagctgtcagcttcacctgc

SEQ ID NO: 51 - RNA, 20 nt, synthetic construct
  source 1..20: mol_type = other RNA
gtcactcataggacaccagt

SEQ ID NO: 52 - RNA, 20 nt, synthetic construct
  source 1..20: mol_type = other RNA
ggtcactcataggacaccag

SEQ ID NO: 53 - RNA, 20 nt, synthetic construct
  source 1..20: mol_type = other RNA
tacccattggcgaccaatga

SEQ ID NO: 54 - RNA, 20 nt, synthetic construct
  source 1..20: mol_type = other RNA
aacacaaaaagatatcagat

SEQ ID NO: 55 - RNA, 20 nt, synthetic construct
  source 1..20: mol_type = other RNA
aaagagtaagccttcacagg

SEQ ID NO: 56 - RNA, 20 nt, synthetic construct
  source 1..20: mol_type = other RNA
gtgtttcttaagattatcta

SEQ ID NO: 57 - protein, 5 aa, synthetic construct
GGGGS

SEQ ID NO: 58 - RNA, 80 nt, synthetic construct
  source 1..80: mol_type = other RNA
gttttagtactctggaaacagaatctactaaaacaaggcaaaatgccgtgtttatctcgtcaacttgttggcgagatttt